SEQUENCE LISTING (2 sequences)

SEQ ID NO: 1 - protein, 25 aa, Artificial Sequence
  Inhibitory peptide of calcineurin
ITSFEEAKGLDRINERMPPRRDAMP

SEQ ID NO: 2 - protein, 16 aa, Artificial Sequence
  VIVT peptide from combinatorial peptide library
MAGPHPVIVITGPHEE

(19) United States
(12) Patent Application Publication
(10) Pub. No.: US 2010/0256082 A1
(43) Pub. Date: Oct. 7, 2010

(54) Metallo-hydrolase inhibitors using metal binding moietes in combination with targeting moieties

(75) Inventor: Robert J. Schotzinger, Raleigh, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Viamet Pharmaceuticals, Inc., Morrisville, NC (US)

(21) Appl. No.: 12/310,709

(22) PCT Filed: Jun. 12, 2007
(86) PCT No.: PCT/US2007/071035

§ 371 Date: Jun. 16, 2009

Related U.S. Application Data
(60) Provisional application No. 60/813,117, filed on Jun. 12, 2006.
(60) Provisional application No. 60/813,105, filed on Jun. 12, 2006.

(51) Int. Cl. A61K 31/52; C07D 473/04; C07D 473/26; C07D 471/04; C07D 471/14; C07D 401/12; C07D 279/16; C07D 265/36; C07H 19/23; C07D 281/06; C07D 487/00; C07D 487/04; G01N 33/53; A61K 31/522; A61K 31/437; A61K 31/519; A61K 31/4439; A61K 31/5415; A61K 31/536; A61K 31/7064; A61K 31/706

(52) U.S. Cl. 514/45; 544/267; 544/265; 544/276; 546/121; 546/82; 544/279; 546/278.1; 544/52; 544/105; 536/27.13; 540/488; 540/350; 544/235; 544/247; 544/230; 544/256; 435/7.6; 514/263.23; 514/263.34; 514/300; 514/293; 514/303; 514/339; 514/224.2; 514/230.5; 514/43; 514/210.09

(57) ABSTRACT

The present invention is directed to methods for screening for metallohydrolase inhibitors using metal binding moieties in combination with targeting moieties.

### CROSS-REFERENCE TO RELATED APPLICATIONS

This application claims the benefit under 35 U.S.C. §119(e) of U.S. Ser. No. 60/813,105, and 60/813,117, both filed Jun. 12, 2006, hereby incorporated by reference in their entireties.

### FIELD OF THE INVENTION

The present invention is directed to methods for screening for metallo-hydrolase inhibitors using metal binding moieties in combination with targeting moieties.

### BACKGROUND OF THE INVENTION

Hydrolases catalyze the hydrolysis of various chemical bonds. They are classified as EC 3 in the EC number classification. One group of hydrolases are metallo-hydrolases that contain at least one metal ion. Some exemplary enzymes in this group are adenosine deaminase, angiotensin converting enzyme, calcineurin, metallo-beta-lactamase, PDE3, PDE4, PDE5, renal dipeptidase, and urease.

Adenosine deaminase (ADA) is a key enzyme in purine metabolism which catalyzes the irreversible deamination of adenosine and deoxyadenosine to inosine and deoxyinosine, respectively. ADA is present in all mammalian cells, and has also been found in a wide variety of microorganisms, plants, and invertebrates. ADA is required for normal development of the lymphoid system. A deficiency in ADA results in varying degrees of immunodeficiency, such as Severed Combined Immunodeficiency (SCID). Cristalli et al., Medical Research Review. 21:105-28 (2001). The metabolic basis of the immunodeficiency is likely related to the sensitivity of lymphocytes to the accumulation of the ADA substrates adenosine and 2′-deoxyadenosine. Blackburn and Kellems, Advance in Immunology. 86:1-41 (2005). In addition, ADA inhibitors have been shown to be useful in hairy cell leukemia, (Pentostatin; see Ann Pharmacother. 1992 July-August; 26(7-8):939-47)

Angiotensin-I-converting enzyme (ACE) is a chloride-dependent metalloenzyme that cleaves a dipeptide from the carboxyl terminus of the decapeptide angiotensin I to form the potent vasopressor (blood vessel constrictor) angiotensin II. It also inactivates the vasodilator bradykinin by sequential removal of two carboxy-terminal dipeptides. There are two forms of ACE in human encoded by a single gene: somatic ACE (sACE) and germinal or testicular ACE (gACE). The structure of the ACE gene is the result of gene duplication; the N and C domains are similar in sequence. Each of the domains contains a catalytically active site characterized by a consensus zinc-binding motif (HEXXH in the single-letter amino-acid code, where X is any amino acid) and a glutamine nearer the carboxyl terminus that also binds zinc; ACE and its homologs therefore make up the M2 gluzincin family. ACE homologs have also been found in other animal species, including chimpanzee, cow, rabbit, mouse, chicken, goldfish, electric eel, house fly, mosquito, horn fly, silk worm, Drosophila melanogaster and Caenorhabditis elegans, and in the bacteria Xanthomonas spp. and Shewanella oneidensis. One human homolog of ACE, ACE2, was identified and shown to be an essential regulator of cardiac function. It differs from ACE in that it contains a single zinc-binding catalytic domain, is a carboxypeptidase with preference for carboxy-terminal hydrophobic or basic residues, and is not affected by ACE inhibitors. Angiotensin I and II, as well as numerous other biologically active peptides, are substrates for ACE2, but bradykinin is not. Riordan, Genome Biology. 4:225 (2003).

Calcineurin is a calcium/calmodulin dependent serine/threonine protein phosphatase, a member of the serine/threonine protein phosphatase family, and has been found in mammals, such as mouse, rat, bovine, and human, as well as other lower eukaryotes many specifies, such as yeast worms, fruit fly and frog. It is a heterodimer consisting of two subunits. CnA and CnB. CnA is the catalytic subunit, consists of a catalytic domain, a CnB-binding domain, and c-terminal regulatory region, which consists of a calmodulin binding domain and an autoinhibitory domain. CnA contains one Zn²⁺ and one Fe³⁺. CnB is Ca²⁺-binding regulatory subunit, containing four Ca²⁺. Rothermel T C M. 13:15-21(2003), Chan et al., PNAS. 102:13075-080 (2005). Calcineurin is a major player in Ca²⁺-dependent eukaryotic signal transduction pathways, involves in many physiological process such as T-cell activation, synaptic plasticity, apoptosis of neurons, development, gene regulation in skeletal and muscle, and cardiac hypertrophy. Changes in intracellular calcium promote binding of calcium and calmodulin to calcineurin to activate it. Activated calcineurin dephosphorylates nuclear factor of activated T cells (NFATs) and promotes its translocation from the cytoplasm to the nucleus, where the NFAT regulates the expression of other transcription factors. Intensive studies have been done with calcineurin since it was first isolated in 1979, mostly due to the ground-breaking discovery that it is the target of two important immunosupressive drugs, FK506 and cyclosporine A (CsA). FK506 and CsA function by complexing with specific cellular immunophilins (FK560 binding protein (FKBP) and cyclophilin A (CypA), respectively) and then bind multiple sites on calcineurin. The binding of CypA-CsA or FKBP-FK506 inhibits the calcium-dependent dephosphorylation of the nuclear factor of activated T cell (NFAT) by calcineurin, thus blocking T cell receptor-mediated cytokine transcription and T-cell activation. However, crystal structures of calcineurin in complex with CypA-CsA or FKBP-FK506 reveal a partial exposed catalytic site in CnB in which the active site residue Arg 122 is at least 10 A from any of the immunophilin and immunosuppressant components. See Chan et al., PNAS 2005, 102:13075-080. Thus, the complexes inhibiting calcineurin activity by sterically preventing calcineurin substrates from binding to the active site. Griffith et al., Cell, 82:507-522 (1995).

Beta-lactamase catalyses the opening and hydrolysis of the beta-lactam ring of beta-lactam antibiotics such as penicillin, cephalosporin and carbapenem to effectively destroy the antibiotic's activity and enable bacteria to survive in the presence of these drugs. This is the major mechanism of resistance to beta-lactam antibiotics in gram-negative bacteria. There are four groups, classed A, B, C and D according to sequence, substrate specificity, and kinetic behavior: class A (penicillinase-type) is the most common. The genes for beta-lactamases are widely distributed in bacteria, frequently located on transmissible plasmids in Gram-negative organisms, although an equivalent chromosomal gene has been found in a few species. Class A, C and D beta-lactamases are serine-utilizing hydrolases. Class B enzymes utilize a catalytic zinc center instead, and requires one or two zinc ion(s) for their activities. Sandanayaka and Prashad, Current Medicinal Chemistry, 9:1145-65 (2002); Daldmon et al, J. Biol. Chem., 278:29240-51 (2003).

Cyclic nucleotide phosphodiesterases (PDEs) are metalloenzymes that hydrolyze the second messenger cyclic AMP and cyclic GMP, to the corresponding 5′ nucleotide monophosphates. The role of PDE enzymes is to regulate intracellular levels of cAMP and cGMP. There are 11 PDE enzymes families (PDE1-PDE11) which have been identified. As these can be derived from multiple genes, many capable of generating a number of isoforms, there currently exists over 50 known PDE enzymes. These enzymes exist as homodimers and there is structural similarity between the different families. However, they differ in several respects including selectivity for cyclic nucleotides, sensitivity for inhibitors and activators, physiological roles and tissue distribution.

PDE3 cyclic nucleotide phosphodiesterases hydrolyze cAMP and cGMP and thereby modulate cAMP- and cGMP-mediated signal transduction. Shakur et al., J. Biol. Chem. 275:38749-61 (2000). These enzymes have a major rote in the regulation of contraction and relaxation in cardiac and vascular myocytes. PDE3 inhibitors, which raise intracellular cAMP and cGMP content, have inotropic effects attributable to the activation of cAMP-dependent protein kinase (PK-A) in cardiac myocytes and vasodilatory effects attributable to the activation of cGMP-dependent protein kinase (PK-G) in vascular myocytes. Shakur et al., Prog. Nucleic Acid Res. Mol. Biol. 66:241-77 (2000). PDE3 involves in regulating contraction and relaxation in cardiac and vascular myocytes, platelet aggregation, anti-lipolytic responses to insulin in adipocytes, insulin secretion by pancreatic β cells and maturation of oocytes. There are two PDE3 genes, PDE3A and PDE3B. PDE3A is expressed primarily in cardiac and vascular myocytes and platelets. PDE3B is expressed primarily in adipocytes, hepatocytes and pancreatic cells (but also in vascular myocytes), and has three isoforms due to alternative splicing. PDE3A1 was cloned from human myocardium and includes all sixteen exons of PDE3A. PDE3A2 was cloned from aortic myocytes and is transcribed from a start site in exon 1. PDE3A3 was cloned from placenta and is transcribed from a start site between exons 3 and 4. U.S. Patent Application Publication 20030158133, herein expressly incorporated by its entirety.

PDE4 enzymes selectively hydrolyze cAMP and have a very low affinity of cGMP. Four genes products of PDE4 (PDE4A-PDE4D) exist, with multiple splice variants. PDE4A, PDE4B and PDE4D are particular abundant in many types of inflammatory and immune cells, including T cells and B cells, monocytes, macrophages, neutrophils and eosinphils. PDE4A, PDE4B and PDE4D are the predominant cAMP-hydrolyzing PDEs in most inflammatory cells and, in general, intracellular increase in cAMP are associated with broad anti-inflammatory effect. See Banner and Trevethick, Trends in Pharmacological Science 25:8 (2004).

PDE5 is a cGMP specific PDE and has been recognized in recent years as an important therapeutic target. It is composed of the conserved C-terminal, zinc containing, catalytic domain, which catalyses the cleavage of cGMP, and an N-terminal regulatory portion, which contains two GAF domain repeats. Each GAF domain contains a cGMP-binding site, one of high affinity and the other of lower affinity. PDE5 activity is regulated through binding of cGMP to the high and low affinity cGMP binding sites followed by phosphorylation, which occurs only when both sites are occupied. Thomas et al. J. Biol. Chem. 265, 14971-14978 (1990). PDE5 is found in varying concentrations in a number of tissues including platelets, vascular and visceral smooth muscle, and skeletal muscle. The protein is a key regulator of cGMP levels in the smooth muscle of the erectile corpus cavemosal tissue. The physiological mechanism of erection involves release of nitric oxide (NO) in the corpus cavemosum during sexual stimulation. NO then activates the enzyme guanylate cyclase, which results in increased levels of cGMP, producing smooth muscle relaxation in the corpus cavemosum and allowing in flow of blood. Inhibition of PDE5 inhibits the breakdown of cGMP allowing the levels of cGMP, and hence smooth muscle relaxation, to be maintained. Corbin & Francis, J. Biol. Chem. 274:13729-32 (1999). Sildenafil (UK-092,480), the active ingredient of Viagra®, and a potent inhibitor of PDE5, has attracted widespread attention for the effective treatment of male erectile dysfunction.

Renal dipeptidase (RDP) is a glycosyiphosphatidyl inositol-anchored enzyme. Its major site of expression is the epithelial cells of the proximal tubule of the kidney. The crystal structure of human renal dipeptidase showed it to be a homodimer with each subunit consisting of a 369 amino acid residue peptide (42 kDa). RDP is a zinc-containing hydrolytic enzyme that shows preference for dipeptide substrates with dehydro amino acids at the carboxyl position. RDP was found to be overexpressed in both benign and malignant tumor compared with normal colonic epithelium. U.S. Patent Application Publication 20050271586, herein expressly incorporated by its entirety.

Urease (urea amidohydrolase) catalyzes the hydrolysis of urea to yield ammonia and carbamate. The latter compound spontaneously decomposes to yield another molecule of ammonia and carbonic acid. The urease phenotype is widely distributed across the bacterial kingdom, and the gene clusters encoding this enzyme have been cloned from numerous bacterial species. Urease synthesis can be nitrogen regulated, urea inducible, or constitutive. Urease is central to the virulence of several human pathogens, such as P. mirabilis and H. pylori. Urea hydrolysis by P. mirabilis in the urinary tract leads directly to urolithiasis (stone formation) and contributes to the development of acute pyelonephritis. The urease of H. pylori is necessary for colonization of the gastric mucosa in experimental animal models of gastritis and serves as the major antigen and diagnostic marker for gastritis and peptic ulcer disease in humans. In addition, the urease of Y. enterocolitica has been implicated as an arthritogenic factor in the development of infection-induced reactive arthritis. Mobley et al., Microbial. Rev. 59: 451-480 (1995). Urease is a Ni enzyme. From studies with the archetypal bacterial urease from Klebsiella aerogenes, Ni is inserted into the apoprotein (UreABC) in a GTP-dependent process that requires the action of UreD, UreF, and UreG and is facilitated by UreE—a putative metallochaperone that delivers Ni. Homologues of ureE are conserved in almost all urease-producing microbes, and cells containing partial ureE deletions exhibit reduced urease specific activities and yield purified enzyme with reduced Ni stoichiometry. Mulrooney et al., J Bacteriol. 187: 3581-85 (2005).

Because many of the hydrolases have been implicated in a variety of diseases, they have been intensely studied as targets for drug development. One of the approach for such drug development is to develop inhibitors of the different kind of hydrolases. One such example is the search for PDE4 inhibitors.

Crystal structure analysis of the catalytic domain PDE4 identifies two metal-binding sites: a high-affinity site and a low-affinity site, which binds one Zn²⁺ ion and one Mg²⁺, respectively. Absolute conservation among the PDEs of two histidine and two aspartic acid residues for divalent metal binding suggests the importance of these amino acids in catalysis. Ke, Implications of PDE4 structure on inhibitor selectivity across PDE families, International Journal for Impotence Research, 16, Suppl. 1: S24-27 (2004). Both metal ions have a role in cAMP hydrolysis. Qing et al., Biochemistry. 42:13220-13226 (2003).

The PDE4 family has also been extensively investigated, as inhibitors of these enzymes are known to be both potent anti-depressants and anti-inflammatory agents. For example, TNF-α has many pro-inflammatory effects, and PDE4 inhibitors are potent suppressors of many cytokines, including TNF-α release from macrophages, monocytes and T cells.

The role of PDE4 inhibitors is currently being investigated in a variety of therapeutic indications including the treatargeting moietyent of inflammatory diseases, such as asthma, chronic obstructive pulmonary disease and psoriasis, as well as treating depression and serving as cognitive enhancers. Houslay et al., Drug Discovery Today, 10:1503-19 (2005).

The first PDE inhibitor to be used therapeutically is theophylline. It is a weak non-selective PDE inhibitor that belongs to a family of xanthine derivatives, which includes 3-isobutyl-1-methylxanthine (IBMX), arofylline, doxofylline and cipamfylline. Although many xanthine derivatives have been developed, and some of them are either under clinical trials (arofylline) or launched (doxofylline), such inhibitors are generally nonselective and relatively weak inhibitors of PDE4. Houslay et al., Drug Discovery Today, 10:1503-19 (2005).

There are also PDE4 selective inhibitors being evaluated. Clinical trials of some of these inhibitors, such as Rolipram, Zardaverine, Filaminast, Mesopram, IC-485 and Piclamilast, proved to be disappointing because of narrow therapeutic windows caused by side effects such as emesis and nausea. There are several additional inhibitors currently in clinical trials, including Atizoram, CC-1088 and ONO-6126. Two inhibitors, trialscilomilast and roflumilast, have completed Phase III clinical trials and are under regulatory review as treatargeting moietyents for asthma and chronic obstructive pulmonary disease. Houslay et al., Drug Discovery Today, 10:1503-19 (2005).

Another PDE4 inhibitor that is in clinical trials is OPC-6535 (tetomilast) by Otsuka Pharmaceutical Co. Ltd. (Zovocio, Drug & Market Development, August: 609-615 (2004)). According to presentations at the Digestive Disease Week 2004 (May 15-19) in New Orleans, La., Otsuka Maryland Research Institute, Inc. (OMRI) began phase III clinical trials in 2003 on the compound OPC-6535 to determine its safety and effectiveness in treating ulcerative colitis. Ulcerative colitis is a chronic digestive disorder, affecting some 500,000 Americans, according to the Crohn's and Colitis Foundation of America. Ulcerative colitis inflames the inner lining of the colon (large intestine) and the rectum. Symptoms range from mild to severe, including persistent diarrhea, abdominal pain, rectal bleeding, fever, weight loss, skin or eye irritations, and delayed growth and sexual maturation in children.

Other PDE4 inhibitors that were or are in clinical trials include AWD-12-28, an indole compound currently in Phase II trials for asthma; YM_{—}976, a pyridopyrimidinone derivative that was discontinued after. Phase I clinical trials; Tofimilast, an indazole derivative in clinical development; Ibudilast, an pyrazolopyridine compound that has been used extensively as an asthma controller in Asian market; and Lirimilast, a benzofuran derivative that was discontinued following a Phase II clinical trial for asthma. Houslay et al., supra.

While some of these candidates have shown promise, there is a need for novel selective inhibitors of metallo-hydrolases, such as PDE4, deaminase, angiotensin converting enzyme, calcineurin, metallo-beta-lactamase, PDE3, PDE5, renal dipeptidase, and urease.

### BRIEF DESCRIPTION OF THE DRAWINGS

FIGS. 1-15 depict a number of metal binding moieties and attachment points, as well as optional derivatives. In all of FIGS. 1-15, R represents the attachment to the other component(s) of the inhibitors of the invention, e.g. the targeting moiety, with an optional linker as described herein. X represents optional individually selected substitution groups, as outlined herein. Z is a heteroatom selected from the group of oxygen, nitrogen and sulfur. As will be appreciated by those in the art, in some cases, the X groups are hydrogen and are generally not depicted. In addition, when non-hydrogen X substitution groups are used, in general, only one X group is preferred. In some cases, and for all the structures herein, as outlined below, two adjacent X groups can be joined to form cyclic structures (including 1 or more cyclic and/or heterocyclic structures, including aromatic).

FIGS. 1A-1AG depicts sulfonyl based metal binding moieties. The class of sulfonyl based metal binding moieties, as for all classes recited herein, can include or exclude any member of the class individually; for example, the depiction of the large number of sulfonyl based metal binding moieties in FIG. 1 can be altered to exclude any member; e.g. “sulfonyl based metal binding moieties except sulfonamide”. Each member can be specifically and independently included or excluded.

FIGS. 2A-2AG depicts carbonyl based metal binding moieties.

FIGS. 3A-3V depicts miscellaneous metal binding moieties. FIGS. 3A-B depict boronic acid based metal binding moieties. It should be noted that in some instances, 3A is not preferred. FIGS. 3C-E depict sulfur based metal binding moieties. FIGS. 3F-3N depict nitrogen based metal binding moieties. FIGS. 3O to 3V depict phosphorus based metal binding moieties.

FIG. 4 depicts a number of metal binding moieties based on 5 membered aromatic heterocycles with one heteroatom.

FIG. 5 depicts a number of metal binding moieties based on 5 membered aromatic heterocycles with two heteroatoms. R and X are as described herein.

FIG. 6 depicts a number of metal binding moieties based 5 membered aromatic heterocycles with three heteroatoms. R and X are as described herein,

FIG. 7 depicts a number of metal binding moieties based 5 membered aromatic heterocycles with four heteroatoms. R and X are as described herein.

FIG. 8 depicts 5 membered non-aromatic rings with 1 heteroatom.

FIG. 9 depicts 5 membered non-aromatic rings with 2 heteroatoms.

FIG. 10 depicts a number of metal binding moieties based on 6 membered aromatic heterocycles with no heteroatom.

FIG. 11 depicts a number of metal binding moieties based on 6 membered aromatic heterocycles with one heteroatom.

FIG. 12 depicts a number of metal binding moieties based on 6 membered aromatic heterocycles with two heteroatoms.

FIG. 13 depicts a number of metal binding moieties based 6 membered aromatic heterocycles with three heteroatoms.

FIG. 14 depicts 6 membered non-aromatic rings with 1 heteroatom.

FIG. 15 depicts 6 membered non-aromatic rings with 2 heteroatoms.

FIG. 16 depicts inhibitors useful as targeting moieties for ADA.

FIG. 17 depicts inhibitors useful as targeting moieties for ACE.

FIG. 18 depicts inhibitors useful as targeting moieties for calcineurin.

FIG. 19 depicts inhibitors useful as targeting moieties for β-lactamase.

FIG. 20 depicts inhibitors useful as targeting moieties for PDE3.

FIG. 21 depicts inhibitors useful as targeting moieties for PDE4.

FIG. 22 depicts inhibitors useful as targeting moieties for PDE5.

FIG. 23 depicts inhibitors useful as targeting moieties for renal dipeptidase.

FIG. 24 depicts inhibitors useful as targeting moieties for urease.

### DETAILED DESCRIPTION OF THE INVENTION

In general, despite the importance of the metal ions to metallo-hydrolase activity, the current evaluation and development of hydrolase inhibitors typically ignores the activity of the metal ions in the design of the inhibitors. For example, a survey of co-crystal structures of PDE4 enzyme with a number of inhibitors show that out of roughly 25 inhibitors, only one was shown to interact directly with the metal ion. Houslay et al., Drug Discovery Today, 10:1503-19 (2005).

The present invention is directed to a two prong approach to inhibiting metallo-hydrolases. As metallo-hydrolases s are a metalloenzyme, the present invention is directed to the combination of a metal binding moiety (MBM) in conjunction with a targeting moiety (TM), linked optionally through a linker. Thus the present invention results in more efficacious inhibitors by combining the affinity and specificity of two different but proximal sites of the metalloprotein.

In this way, both additive and synergistic binding effects, including both binding affinity and binding specificity, can be utilized. As will be appreciated by those in the art, this can work in a variety of different ways. Some metal binding moieties, such as the hydroxamates, bind tightly to zinc ions, for example. However, these inhibitors tend to be not very specific, and can exhibit toxic effects from binding zinc in a variety of metalloproteins. The present invention provides for “extra” specificity of tight metal binding moieties by using specificity to the region of the metalloprotein in proximity to the metal binding site to allow for better targeting and a reduction in toxicity due to non-specific binding. Similarly, the addition of two moieties with low affinity and/or low specificity can result in an inhibitor with high affinity and/or high specificity. Thus, any combination of “good” and “poor” Metal binding moieties can be linked to either “good” or “poor” targeting moieties to result in “good” inhibitors.

The invention provides a variety of aspects. In one embodiment, the invention provides inhibitors comprising a metal binding moieties and a targeting moiety, again, optionally with linkers. In an additional aspect, the invention provides methods of screening for inhibitors of metallo-hydrolases using metal binding moieties in combination with targeting moieties and optional linkers.

Inhibitors of Metallo-Hydrolases

The present invention provides inhibitors of metallo-hydrolases comprising one or more metal binding moieties, a targeting moiety, and optionally a linker,

By “inhibitor” herein is meant a molecule that is capable of inhibiting metallo-hydrolase. By “inhibit” herein meant to decrease the activity of the metallo-hydrolase, as compared to the activity in the absence of the inhibitor. In this case, “inhibit” is generally at least a 5-20-25% decrease in the activity, with over 50-75% being useful in some embodiments and a 95-98-100% loss of activity being useful as well. The activity of each metallo-hydrolase may vary, and is described in more details herein. Assay for measuring individual activity is described below.

### Metal Binding Moieties

By “metal binding moiety (MBM)” herein is meant a moiety that is capable of binding to metal ions through one or more coordination atoms of the MBM, resulting in a coordinate/covalent attachment of the metal to the coordination atom(s). In general, this is due to at least one pair of unpaired electrons. As is appreciated by those in the art, the nature of the coordination bond can have covalent characteristics but is generally referred to as a “coordinate” or “coordinate/covalent” bond.

In some embodiments, the metal binding moieties provides a single coordination atom for binding to the metal ion of metallo-hydrolases, such as the zinc ion of the PDE4 molecule; in other embodiments, two or more coordination atoms are provided by the metal binding moieties. When two or more coordination atoms are provided by the metal binding moieties, the metal binding moieties can be referred to as a “chelator” or a “ligand”. The number of coordination sites is an intrinsic characteristic of the metal being bound: those molecules that use two different atoms to form two bonds to a metal are said to be bidentate. The terms tridentate, tetradentate, pentadentate, etc. then refer to metal binding moieties that use three, four and five atoms to form the same number of bonds respectively.

In general, the nature of the coordination atom depends on the metal to be bound. In general, useful heteroatoms for use as coordination atoms include nitrogen, oxygen and sulfur.

As will be appreciated by those in the art, a wide variety of suitable metal binding moieties can be used. The metal binding moieties can be macrocyclic or non-macrocyclic in structure. “Macrocyclic” in this context includes means at least 12 atoms in a cyclic structure, frequently containing heteroatoms, binding of a metal in the interior of the cycle and generally planar.

In many embodiments, the metal binding moieties are not macrocyclic, but may contain cyclic structures.

One class of suitable metal binding moieties are five membered ring structures with at least one heteroatom and can be aromatic or non-aromatic. Subclasses of this class include, but are not limited to, five membered rings with 1 heteroatom (51HA), including five membered aromatic rings with 1 heteroatom (5A1HA) and five membered non-aromatic rings with 1 heteroatom (5NA1HA); five membered rings with 2 heteroatoms (again, either aromatic or not: 5A2HA and 5NA2HA); five membered rings with three heteroatoms (either aromatic or not, 5A3HA and 5NA3HA) and five membered aromatic rings with 4 heteroatoms (5A4HA). As outlined above, each class or subclass can include or exclude any member of the class or subclass individually. Additionally, each heteroatom can be included or excluded independently and individually as well; for example, the five membered aromatic ring with 1 heteroatom may exclude nitrogen as the heteroatom.

Another class of suitable metal binding moieties are six membered ring structures with none or at least one heteroatom that can be aromatic or non-aromatic. Subclasses of this class include, but are not limited to, six membered aromatic rings with no heteroatoms (6A), six membered rings with 1 heteroatom (61HA), including six membered aromatic rings with 1 heteroatom (6A1HA) and six membered non-aromatic rings with 1 heteroatom (6NA1HA); six membered rings with 2 heteroatoms (again, either aromatic or not: 6A2HA and 6NA2HA); six membered rings with three heteroatoms (either aromatic or not, 6A3HA and 6NA3HA). As outlined above, each class or subclass can include or exclude any member of the class or subclass individually. Additionally, as for the five membered ring structures, each heteroatom can be included or excluded independently as well.

It should be noted that in the case where adjacent substitution groups form a cyclic structure, the actual metal binding moiety may be based on a 5 or 6-membered ring but include additional ring structures.

As depicted in the Figures, one group of suitable metal binding moieties are the class of sulfonyl based metal binding moieties, including, but not limited to, sulfonic acid, sulfonamide, thiosulfonic acid, sulfonyl hydrazide, sulfonyl hydroxylamine, N-methoxy-sulfonamide, N-alkylamino-sulfonamide, N-acetyl sulfonyl hydrazide, N-carboxamide sulfonylhydrazide, N-cyanosulfonamide, cyanomethyl-sulfonamide, N-acetyl sulfonamide, uridosulfonamide, thiouredosulfonamide, guanidylsulfonamide, sulfonyl-thioacetamide, sulfonylacetamide, sulfonylmethyl-phosphonic acid, methylcyano sulfonamide, sulfonyl glycinamide, sulfonyl carboximidamide, O-acetyl sulfonyl hydroxylamine, sulfonylimidazole, sulfonylpyrazole, sulfonyl-1,2,4-triazole, sulfonyl-1,2,3-triazole, sulfonyl pyrrolidin-2-one, sulfonyl imidazolidione, sulfonyl hydantoin, sulfonyl, sulfonyl piperazine, sulfonyl morpholine.

As depicted in the Figures, one group of suitable metal binding moieties are carbonyl based, including, but not limited to, carboxylic acid, amide, thiocarboxylic acid, thioamide, carboxamidine, oxime, nitrite, hydroxamic acid, N-alkyl hydroxamic acid, O-alkyl hydroxamic acid, N,O-dialkyl hydroxamic acid, carboximidamide, carboxhydrazine, substituted carbohydrazide, N-hydroxy carboxhydrazide, N-acyl carboxamide, carboxpyrrolidinone, N-cyano carboxamide, carboxyurea, carboxythiourea, N-amidino carboxyamide, carboximidazolidine, carbox-thioimidazoline, acylacetamide, carbox-methylphosphonic acid, carbamoyl methyl ester, glycinamide, carboxylic acid carboxymethyl ester, N-cyanomethyl carboxamide, acylpiperazine, acylpiperazin-3-one.

As depicted in the Figures, one group of the metal binding moieties are boron based, including, but not limited to, boronic acid.

As depicted in the Figures, one group of the metal binding moieties are sulfur based, including, but not limited to, thiol, 1,3-dithiolane, and 1,3-dithiolane.

As depicted in the Figures, one group of metal binding moieties are nitrogen based, including, but not limited to, N-acetyl-N-hydroxy amine, acetohydroxamic acid methyl ester, carbamate, urea, guanidine, 2-oxothiazolidine, hydroxy urea.

As depicted in the Figures, one group of metal binding moieties are phosphorus based, including, but not limited to, phosphonic acid, thiophosphonic acid, phosphoric acid, phosphate, thiophosphate, phosphonoamine, phosphoramide, and thiophosphoramide.

As depicted in the Figures, one group of metal binding moieties are 5-member, 1-hetero aromatic compounds, including, but not limited to, pyrrole, furan, and thiophene.

In some embodiments, the heteroatom in the 5-member, 1-hetero aromatic compounds is not oxygen.

In some embodiments, the heteroatom in the 5-member, 1-hetero aromatic compounds is not nitrogen.

In some embodiments, the heteroatom in the 5-member, 1-hetero aromatic compounds is not sulfur.

As depicted in the Figures, one group of metal binding moieties are 5-member, 2-hetero aromatic compounds, including, but not limited to, N-alkyl Imidazole, N-Alkyl Substituted Imidazole, imidazole, oxazole, thiazole, N-substituted pyrazole, pyrazole, isoxazole, and isothiazole.

In some embodiments, one of the hetero atom in the 5-member, 2-hetero aromatic compounds is not oxygen.

In some embodiments, neither heteroatom in the 5-member, 2-hetero aromatic compounds is oxygen.

In some embodiments, one of the heteroatom in the 5-member, 2-hetero aromatic compounds is not nitrogen.

In some embodiments, neither heteroatom in the 5-member, 2-hetero aromatic compounds is nitrogen.

In some embodiments, one of the heteroatom in the 5-member, 2-hetero aromatic compounds is not sulfur.

In some embodiments, neither heteroatom in the 5-member, 2-hetero aromatic compounds is sulfur.

When the hydrolase is HIV protease, in some cases unsubstituted thiazole is not preferred as the metal binding moiety, and in some cases thiazole is not preferred.

When the hydrolase is PDE4, PDE5, ACE, caspase, or carboxy peptidase, the metal binding moiety in some cases the metal binding moiety is not N-substituted pyrazole.

When the hydrolase is PDE4, PDE5, ACE, caspase, or carboxy peptidase; in some cases the metal binding moiety is not unsubstituted N-substituted pyrazole.

As depicted in the Figures, one group of metal binding moieties are 5-member, 3-hetero aromatic compounds, including, but not limited to, 1,2,3-triazole, substituted 1,2,4-triazole, 1,2,4-oxadiazole, 1,2,4-thiazole, 1,3,4-oxadiazole, 1,3,4-thiadiazole, N-substituted 1,2,3-triazole, 1,2,3-triazole, 1,2,3-oxadiazole, 1,2,3-thiadiazole, 2,1,3-oxadiazole, and 2,1,3-thiadiazole.

In some embodiments, one of the heteroatom in the 5member, 3-hetero aromatic compounds is not oxygen.

In some embodiments, two of the heteroatoms in the 5-member, 3-hetero aromatic compounds are not oxygen.

In some embodiments, none of the heteroatoms in the 5-member, 3-hetero aromatic compounds is oxygen.

In some embodiments, one of the heteroatoms in the 5-member, 3-hetero aromatic compounds is not nitrogen.

In some embodiments, two of the heteroatoms in the 5-member, 3-hetero aromatic compounds are not nitrogen.

In some embodiments, none of the heteroatoms in the 5-member, 3-hetero aromatic compounds is nitrogen.

In some embodiments, one of the heteroatoms in the 5-member, 3-hetero aromatic compounds is not sulfur.

In some embodiments, two of the heteroatoms in the 5-member, 3-hetero aromatic compounds are not sulfur.

In some embodiments, none of the heteroatoms in the 5-member, 3-hetero aromatic compounds is not sulfur.

As depicted in the Figures, one group of metal binding moieties are 5-member, 4-hetero aromatic compounds, including, but not limited to, 1,2,3,4-tetrazole, 1,2,3,4-oxatriazole, and 1,2,3,4-thiatriazole.

In some embodiments, one of the heteroatoms in the 5-member, 4-hetero aromatic compounds is not oxygen.

In some embodiments, two of the heteroatoms in the 5-member, 4-hetero aromatic compounds are not oxygen.

In some embodiments, three of the heteroatoms in the 5-member, 4-hetero aromatic compounds are not oxygen.

In some embodiments, none of the heteroatoms in the 5-member, 4-hetero aromatic compounds is oxygen.

In some embodiments, one of the heteroatoms in the 5-member, 4-hetero aromatic compounds is not nitrogen.

In some embodiments, two of the heteroatoms in the 5-member, 4-hetero aromatic compounds are not nitrogen.

In some embodiments, three of the heteroatoms in the 5-member, 4-hetero aromatic compounds are not nitrogen.

In some embodiments, none of the heteroatoms in the 5-member, 4-hetero aromatic compounds is nitrogen.

In some embodiments, one of the hetero atom in the 5-member, 4-hetero aromatic compounds is not sulfur.

In some embodiments, two of the heteroatoms in the 5-member, 4-hetero aromatic compounds are not sulfur.

In some embodiments, three of the heteroatoms in the 5-member, 4-hetero aromatic compounds are not sulfur.

In some embodiments, none of the heteroatoms in the 5-member, 4-hetero aromatic compounds is sulfur.

As depicted in the Figures, one group of metal binding moieties are 6-member, 0-hetero aromatic compounds, including, but not limited to, ortho-substituted benzene.

As depicted in the Figures, one group of metal binding moieties are 6-member, 1-hetero aromatic compounds, including, but not limited to, pyridine.

As depicted in the Figures, one group of metal binding moieties are 6-member, 2-hetero aromatic compounds, including, but not limited to, pyridazine, pyrimidine, and pyrazine.

As depicted in the Figures, one group of metal binding moieties are 6-member, 3-hetero aromatic compounds, including, but not limited to, 1,2,4-triazine and 1,3,5-triazine.

As depicted in the Figures, one group of metal binding moieties are 5-member, 1-hetero non-aromatic compounds, including, but not limited to, pyrrolidinone, 3-hydroxy pyrrolidinone, succinimide, maleimide, N-hydroxy pyrrolidinone, butyrolactone, 3-hydroxy butyrolactone, thiobutyrolactone, and 3-hydroxy butyrolactone.

In some embodiments, the heteroatom in the 5-member, 1-hetero non-aromatic compounds is not oxygen.

In some embodiments, the heteroatom in the 5-member, 1-hetero non-aromatic compounds is not nitrogen.

In some embodiments, the heteroatom in the 5-member, 1-hetero non-aromatic compounds is not sulfur.

As depicted in the Figures, one group of metal binding moieties are 5-member, 2-hetero non-aromatic compounds, including, but not limited to, pyrazolone, imidazolidine, hydantoin, thiazolonone, thiazolidinine, oxazolidone, and oxazolidoine-2,4-dione.

In some embodiments, one of the hetero atom in the 5-member, 2-hetero non-aromatic compounds is not oxygen.

In some embodiments, neither heteroatom in the 5-member, 2-hetero non-aromatic compounds is oxygen.

In some embodiments, one of the heteroatom in the 5-member, 2-hetero non-aromatic compounds is not nitrogen.

In some embodiments, neither heteroatom in the 5-member, 2-hetero non-aromatic compounds is nitrogen.

In some embodiments, one of the heteroatom in the 5-member, 2-hetero non-aromatic compounds is not sulfur.

In some embodiments, neither heteroatom in the 5-member, 2-hetero non-aromatic compounds is sulfur.

As depicted in the Figures, one group of metal binding moieties are 6-member, 1-hetero non-aromatic compounds, including, but not limited to, N-hydroxy pyridine, and 3-hydroxy pyridine.

In some embodiments, the heteroatom in the 6-member, 1-hetero non-aromatic compounds is not oxygen.

In some embodiments, the heteroatom in the 6-member, 1-hetero non-aromatic compounds is not nitrogen.

In some embodiments, the heteroatom in the 6-member, 1-hetero non-aromatic compounds is not sulfur.

As depicted in the Figures, one group of metal binding moieties are 6-member, 2-hetero non-aromatic compounds, including, but not limited to, pyridazin-3(2H)-one, dioxopyridazine, glutarimide, 2,6-dioxopyrimidine, 3-oxopiperazine, morpholinone, 2,3-dioxopiperazine, and 2,5-dioxopiperazine.

In some embodiments, one of the hetero atom in the 6-member, 2-hetero non-aromatic compounds is not oxygen.

In some embodiments, neither heteroatom in the 6-member, 2-hetero non-aromatic compounds is oxygen.

In some embodiments, one of the heteroatom in the 6-member, 2-hetero non-aromatic compounds is not nitrogen.

In some embodiments, neither heteroatom in the 6-member, 2-hetero non-aromatic compounds is nitrogen.

In some embodiments, one of the heteroatom in the 6-member, 2-hetero non-aromatic compounds is not sulfur.

In some embodiments, neither heteroatom in the 6-member, 2-hetero non-aromatic compounds is sulfur

As shown in the Figures, the metal binding moieties have an attachment site, generally depicted as “R”, which is used to attach the targeting moiety, described below, optionally using a linker.

As depicted in the Figures, in addition to the attachment site, many of the metal binding moieties can be optionally derivatized, for example as depicted using an “X” substitution group. In some cases these X groups can provide additional coordination atoms. Suitable substitution groups are known in the art and include, but are not limited to, hydrogen, linkers (usually depicted herein as “L” or “Lₙ”, with n being 0 or 1) alkyl, alcohol, aromatic, amino, amido, carbonyl, carboxyl, cyano, nitro, ethers, esters, aldehydes, sulfonyl, silicon moieties, halogens, sulfur containing moieties, phosphorus containing moieties, and ethylene glycols. In the structures depicted herein, X is hydrogen when the position is unsubstituted. It should be noted that some positions may allow two substitution groups, X and X′, in which case the X and X′ groups may be either the same or different. Generally, in some embodiments, only a single non-hydrogen X group is attached at any particular position; that is, preferably at least one of the X groups at each position is hydrogen. Thus, if X is an alkyl or aryl group, there is generally an additional hydrogen attached to the carbon, although not necessarily depicted herein. In addition, X groups on adjacent carbons can be joined to form ring structures (including heterocycles, aryl and heteroaryls), which can be further derivatized as outlined herein.

By “alkyl group” or grammatical equivalents herein is meant a straight or branched chain alkyl group, with straight chain alkyl groups being preferred. If branched, it may be branched at one or more positions, and unless specified, at any position. “Alkyl” in this context includes alkenyl and alkynyl, and any combination of single, double and triple bonds. The alkyl group may range from about 1 to about 30 carbon atoms (C1-C30), with a preferred embodiment utilizing from about 1 to about 20 carbon atoms (C1-C20), with about C1 through about C12 to about C15 being preferred, and C1 to C5 being particularly preferred, although in some embodiments the alkyl group may be much larger. Also included within the definition of an alkyl group are cycloalkyl groups such as C5 and C6 rings, and heterocyclic rings with nitrogen, oxygen, sulfur or phosphorus, as well as cycloalkyl and heterocycloalkyl groups with unsaturated bonds. Alkyl also includes heteroalkyl, with heteroatoms of sulfur, oxygen, nitrogen, and silicone being preferred. Alkyl includes substituted alkyl groups. By “substituted alkyl group” herein is meant an alkyl group as defined herein further comprising one or more substitution moieties “X”, as defined herein.

By “amino groups” or grammatical equivalents herein is meant —NH₂, —NHX and —NX₂ groups, with X being as defined herein.

By “nitro group” herein is meant an —NO₂ group.

By “sulfur containing moieties” herein is meant compounds containing sulfur atoms, including but not limited to, thia-, thio- and sulfo-compounds, thiols (—SH and —SX), and sulfides (—XSX—). By “phosphorus containing moieties” herein is meant compounds containing phosphorus, including, but not limited to, phosphines and phosphates. By “silicon containing moieties” herein is meant compounds containing silicon.

By “ether” herein is meant an —O—X group. Preferred ethers include alkoxy groups, with —O—(CH₂)₂CH₃ and —O—(CH₂)₄CH₃ being preferred.

By “ester” herein is meant a —COOX group, including carboxyl groups. By “carboxyl” herein is meant a —COON group.

By “halogen” herein is meant bromine, iodine, chlorine, or fluorine. Preferred substituted alkyls are partially or fully halogenated alkyls such as CF₃, etc.

By “aldehyde” herein is meant —XCOH groups.

By “alcohol” herein is meant —OH groups, and alkyl alcohols —XOH.

By “amido” herein is meant —XCONH— or XCONX— groups.

By “ethylene glycol” or “(poly)ethylene glycol” herein is meant a —(O—CH₂—CH₂)ₙ— group, although each carbon atom of the ethylene group may also be singly or doubly substituted, i.e. —(O—CX₂—CX₂)ₙ—, with X as described above. Ethylene glycol derivatives with other heteroatoms in place of oxygen (i.e. —(N—CH₂—CH₂)ₙ— or —(S—CH₂—CH₂)ₙ—, or with substitution groups) are also useful.

By “aryl group” or grammatical equivalents herein is meant an aromatic monocyclic or polycyclic hydrocarbon moiety generally containing 5 to 14 carbon atoms (although larger polycyclic rings structures may be made) and any carbocyclic ketone or thioketone derivative thereof, wherein the carbon atom with the free valence is a member of an aromatic ring. Aromatic groups include arylene groups and aromatic groups with more than two atoms removed. For the purposes of this application aromatic includes heteroaryl. “Heteroaryl” means an aromatic group wherein 1 to 5 of the indicated carbon atoms are replaced by a heteroatom chosen from nitrogen, oxygen, sulfur, phosphorus, boron and silicon wherein the atom with the free valence is a member of an aromatic ring, and any heterocyclic ketone and thioketone derivative thereof. Thus, heteroaryl includes for example pyrrolyl, pyridyl, thienyl, or furanyl (single ring, single heteroatom); oxazolyl, isoxazolyl, oxadiazolyl, or imidazolyl (single ring, multiple heteroatoms); benzoxazolyl, benzothiazolyl, or benzimidazolyl, (multi-ring, multiple heteroatoms); quinolyl, benzofuranyl or indolyl (multi-ring, single heteroatom). “Aryl” includes substituted aryl and substituted heteroaryl groups as well, with one or more X groups as defined herein.

X substituents can be used to modify the solubility of the candidate inhibitors, or alter the electronic environment of the metal binding moiety. For example, additional selected ring substituents are utilized to alter the solubility of the resulting candidate inhibitor in either aqueous or organic solvents. Typically, the substitution of alkyl, alkoxy, perfluoroalkyt, CN, amino, alkylamino, dialkylamino, 1-(acyloxy)alkylester of carboxy, aryl or heteroaryl onto the metal binding moiety results in an candidate inhibitor that is more soluble in non-polar solvents. Alternatively, substitution is by a “water solubilizing group”, i.e. a sulfonic acid, salt of sulfonic acid, salt of amine, carboxy, carboxyalkyl, carboxyalkoxy, carboxyalkylamino, or carboxyalkylthio or other substituent that results in a candidate inhibitor that is more soluble in aqueous solution. Similarly, careful selection of the identity of linker and targeting moiety is also used to modify the solubility of the final candidate inhibitor with those candidate inhibitors containing charged or ionizable groups usually enhancing water solubility.

Alternatively, a ring substituent is used as a reactive site to further modify candidate inhibitors to attach the candidate inhibitors to a carrier or substrate as is more fully outlined below.

A number of suitable metal binding moieties are depicted in the Figures.

### Targeting Moieties

In addition to the metal binding moieties, the inhibitors of the invention comprise targeting moieties. By “targeting moiety” herein is meant a functional group that serves to target or direct the inhibitor to a particular location or association. Thus for example, a targeting moiety may be used to bring the metal binding moiety to the vicinity of a metal ion that is essential to the function of metallo-hydrolases such as PDE4 enzymes. That is, the targeting moiety has binding affinity and/or binding specificity for the PDE4 enzyme, preferably in proximity of the metal binding site, such that the metal binding moieties can bind the metal ion. As described below, optional linkers are used to provide proper spacing.

In general, one class of suitable targeting moieties are those that are or have been shown to be inhibitors of the metallo-hydrolases of the present invention, some of which are described or depicted in the figures. It should be noted that the basis for many of the targeting moieties of the Figures are inhibitors that have been co-crystallized with the hydrolases, and certain moieties of the inhibitors have been replaced with an optional linker and an metal binding moiety.

In addition, substrates and modified substrates can also be used. In some cases, the use of the substrates for recruitment of the compositions of the invention to the metallo-hydrolases allows inhibition due to metal binding, even if the substrate is cleaved.

In general, these targeting moieties contain at least one substituent that comprises the attachment linker and the metal binding moiety. In general, one of skill in the art can determine the appropriate substitution location. For example, substituents off of ring components can be done. Saturated carbon atoms can also be substituted. Similarly, functional groups (e.g. amino, carboxy, hydroxy, etc.) that are not involved in activity of the targeting moiety can be used as attachment locations. Below are some of the metallo-hydrolases and their particular inhibitors.

### Adenosine Deaminase

Adenosine deaminase (ADA) (EC 3.5.4.4, also known as adenosine aminhydrolase and deoxyadenosine deaminase)) has an α/β barrel structural motif, with eight β-strands and eight peripheral a-helices. Sharff et al. J. Mol. Biol. 226:917-21(1992). The α-helices are connected by the b-strands in a βαβ arrangement. The active site of ADA is located at the C-terminal end of the βbarrel in an oblong shaped pocket, with a Zn²⁺ cofactor embedded in the deepest part of the pocket. Wang and Quiocho Biochemistry 37:8314-24 (1998). HDPR (6(R)-hydroxyl-1,6-dihydropurine ribonucleoside) is the ligand bound in the active site. Wilson et al. Biochemistry, 32:1689-94 (1993). HDPR is regarded as a nearly ideal transition-state analogue. Wang and Quiocho, supra.

The Zn²⁺ is pentacoordinated to the side chains of His 15, His 17, His 214, Asp 295, and the 6-hydroxy of HDPR. Wilson et al. supra. All of the Zn²⁺ coordinating residues also participate in other interactions. Hydrogen bonds are formed between His 15 and Glu 260, His 17 and HDPR, His 214 and Asp181, and Asp295 and Ser 265. Wang and Quiocho, supra. Interactions between ADA and HDPR revealed that Glu 217 and Asp 296 have higher pKa values than what would normally be expected. One explanation for the high pKa values is the hydrophobic nature of Leu 58, Phe 61, Phe 62, and Phe 65, in the surrounding environment. The ionization states of the residues also play a part in the catalytic mechanism. Sharff et al, supra.

An ADA catalyzed reaction includes the involvement of Glu 217, His 238, Asp 295. and Zn²⁺. The reaction involves two stages which include the addition of the initial stereospecific hydroxide to the C6 of the substrate to yield a transition-state intermediate, and a final ammonia elimination to yield the inosine product. Wang and Quiocho, supra. Leu 106 has the ability to interact directly with the substrate of ADA, but Tyr 97 is about 20 angstroms away from ADA's active site. Therefore, it is impossible for Tyr 97 to have any direct contact with the substrates in the active site. However, the presence of charged Glu 99, Arg 235, and Glu 260 build a salt bridge linking Tyr 97 to the active site of ADA. The bridge is completely buried in the center of the b-barrel, and plays a major role in the reaction catalyzed by the enzyme. Jiang et al., Human Molecular Genetics 6:2271-78 (1997).

Comparisons of ADA structures in humans, mice, cows, E. coli, and myco-bacterium were made. Fifteen of the twenty amino acids were found in the same location for all five of these structures. These amino acids include praline, histidine, threonine, leucine, alanine, glutamic acid, tyrosine, aspartic acid, serine, cysteine, glycine, valine, arginine, asparagine, and phenylalanine. When only comparing the structures of humans, mice and cows, all twenty amino acids were found in the same position for aft three of these structures. According to Wilson et al., each mammalian ADA has similarities in their substrate specificity, and activity and sensitivity to inhibitors by various compounds. Human ADA is only 11 residues longer than murine ADA, and there are no gaps in the superimposed sequences of both structures. The sequences of human and murine ADA is very similar, with 83% of the superimposed residues having identical sidechains. The differences in amino acids that are found are conservative in nature, and don't have an effect on the activity. Residues that are directly or indirectly associated with the binding site of murine ADA are superimposed with the same residues in the ADA sequence of humans. The residues that undergo point mutations in human ADA are also identical to the residues in murine ADA.

There are a wide variety of suitable targeting moieties. ADA inhibitors that have been tested include, but are not limited to, pentostatin (hairy cell leukemia; see Ann Pharmacother. 1992 July-August; 26(7-8):939-47, incorporated by reference); deoxycoformycin, sometimes used in conjunction with arabinofuranosyladenine (acute leukemia patients; see Recent Results Cancer Res. 1982;80:323.30); coformycin and analogues (Hosmane R. in Modified Nucleosides, Synthesis, and Applications, Loakes, D, Ed., Transworld Research Network, Trivendrum, 2002; pp. 133-151); a number of 1- and 2-alkyl derivatives of the 4-aminopyrazolo[3,4-d]pyrimidine (APP) nucleus (see J Med Chem. 2005 Aug. 11; 48(16):5162-74); heterocyclic derivatives for antiviral activity (see J Infect Dis. 1975 June; 131(6):673-7), all of which are expressly incorporated by reference, and in particular for the compounds they disclose, useful as targeting moieties herein.

The Figures depicts a number of clinically tested inhibitors of ADA, which are suitable for use as targeting moieties in the present invention. The Figure shows the structure of these known inhibitors along with possible sites of attachment of the linkers and metal binding moieties (“R”), as well as possible derivatives.

In addition to these targeting moieties, other known targeting moieties, identified by the screens outlined below or shown to bind to ADA can be used. Thus, suitable targeting moieties include, but are not limited to, small organic molecules including known drugs and drug candidates, polysaccharides, fatty acids, vaccines, polypeptides, proteins (including peptides, as described herein), nucleic acids, carbohydrates, lipids, hormones including proteinaceous and steroid hormones, growth factors, receptor ligands, antigens, antibodies and enzymes, (as outlined below, “candidate agents” are included) etc.

ADA activity can be measured using established method. See e.g. Guisti G, Galanti B: Adenosine deaminase: colorimetric method. In Methods of Enzymatic Analysis. 5th edition. Edited by: Bergmeyer H U. Weinheim (Germany): Verlag Chemie; 1984:315-323; Murphy et al., Anal. Biochemistry 122, 328-337; and Trotta et al., PNAS, 73:104-108 (1976), herein incorporated by reference.

### Angiotensin Converting Enzyme

The metallopeptidase Angiotensin Converting Enzyme (ACE, EC 5.4.15.1, also known as peptidyl-dipeptidase A, kininase II, dipeptidyl carboxypeptidase I; peptidase P; carboxycathepsin; dipeptide hydrolase; peptidyl dipeptidase; angiotensin converting enzyme; kininase II; angiotensin I-converting enzyme; carboxycathepsin; dipeptidyl carboxypeptidase; peptidyl dipeptidase I; peptidyl-dipeptide hydrolase; peptidyldipeptide hydrolase; endothelial cell peptidyl dipeptidase; peptidyl dipeptidase-4; peptidyl dipeptidase A; PDH; peptidyl dipeptide hydrolase; DCP)) is an important drug target for the treatment of hypertension, heart, kidney, and lung disease. Since the 1980s, inhibitors of angiotensin converting enzyme (ACE inhibitors) have achieved great success as first-line therapy for cardiovascular diseases, including high blood pressure, heart failure, coronary artery disease, and kidney failure. These anti-hypertensive drugs were not designed based on any knowledge of the three-dimensional structure of ACE, but on an assumed mechanistic homology to carboxypeptidase A, whose structure has been known for some time. However, prolonged administration of current ACE inhibitors leads to several undesirable side effects, such as a persistent dry cough, headaches and dizziness. There are two isoforms of ACE in the human body, somatic and testicular. Somatic ACE exists in most cells in the body and testicular ACE, which is half the size of somatic ACE, is found only in the testes. Both convert inactive angiotensin I to its active form, angiotensin II, which stimulates blood vessel constriction. Both forms of ACE also inactivate bradykinin, which stimulates blood vessel dilation. The three-dimensional structure reveals that ACE is composed of a-helices for the most part, and incorporates a zinc ion and two chloride ions. In fact it bears little resemblance to carboxypeptidase A except in the active site zinc-binding motif. Instead, it resembles rat neurolysin and Pyrococcus furiosus carboxypeptidase, despite sharing little amino-acid sequence similarity with these two proteins. This similarity extends to the active site, which consists of a deep, narrow channel that divides the molecule into two subdomains. On top of the molecule is an amino-terminal ‘lid’, which seems to allow only small peptide substrates (2530 amino acids) access to the active site cleft this accounts for the inability of ACE to hydrolyse large, folded substrates. Natesh et al., Nature 421:551-54 (2003).

There are a wide variety of suitable targeting moieties, including, but not limited to, Alacepril (Cetapril, Dainippon), Benazepril; Captopril; Cilazapril; Delapril; Enalapril; Fosinopril; Imidapril; Lisinopril; Moexipril; Perindopril (Coversal); Quinapril (Accupril); Ramipril; Trandolapril (Trodoprii); Spirapril; and Enalaprilat. The Figures depict a number of inhibitors of angiotensin converting enzyme, which are suitable for use as targeting moieties in the present invention, and show the structure of these known inhibitors along with possible sites of attachment of the linkers and metal binding moieties, as well as possible derivatives.

In addition to these targeting moieties, other known targeting moieties, identified by the screens outlined below or shown to bind to angiotensin converting enzyme can be used. Thus, suitable targeting moieties include, but are not limited to, small organic molecules including known drugs and drug candidates, polysaccharides, fatty acids, vaccines, polypeptides, proteins (including peptides, as described herein), nucleic acids, carbohydrates, lipids, hormones including proteinaceous and steroid hormones, growth factors, receptor ligands, antigens, antibodies and enzymes, (as outlined below, “candidate agents” are included) etc.

ADA activity can be measured using established method. See e.g. Kapiloff et al., Anal Biochem, 140:293-302 (1984); Friedland and Silverstein, Am J Clin Pathol. 66: 416-424 (1976); and Kasahara et al., Clin Chem. 27:1922-1925 (1981), herein incorporated by reference.

### Calcineurin

Calcineurin (protein phosphatase 2B, EC 3.1.3.16), the only serine/threonine phosphatase under the control of Ca2+/calmodulin, is an important mediator in signal transmission, connecting the Ca2+-dependent signaling to a wide variety of cellular responses.

As substrates of calcineurin, transcription factors of the NFAT family play an essential role in lymphocyte activation, and it follows that their function is also inhibited by CsA and FK506. Although the use of these drugs has been crucial for the success of organ transplantation, their therapeutic use is associated with severe side effects. There is, therefore a need to develop better, less toxic immunosuppressive agents. Martinez-Martinez, and Redondo, Inhibitors of the calcineurin/NFAT pathway, Current Medicinal Chemistry, 11:997-1007 (2004).

Besides the well-known inhibitors such as CsA and FK506, there are a number of natural products have been isolated that are potent inhibitors of calcineurin and other serin/threonine protein phosphatases. See Rusnak and Mertz, Calcineurin: form and function, Physiological Review, 80:1483-1521 (2000). There are a wide variety of suitable targeting moieties. Okadaic acid is s potent and specific inhibitor of PP2A, and can inhibit calcineurin at higher concentrations; microcycstin LR is a cyclic peptide and is a relatively potent calcineurin inhibitor; dibefurin is a fugal metabolite with modest inhibitory activity against calcineurin. In addition, there also synthetic compounds that have been found to be reasonable inhibitors of calcineurin and other phosphatases.

One group is endothal derivatives. Endothal is structurally related to the natural defensive toxin of blister beetles, cantharidin, a potent inhibitor of PP1 and PP2A, but a weak inhibitor of calcineurin. Computational modeling shows that the tethered dicarboxylic acid moiety and bridgehead oxygen atom of endothall and cantharidin derivatives interact with the active site dinuclear metal center. See Tatlock et al., Structure-based design of novel calcineurin (PP2B) inhibitors, Bioorganic and Medicinal Chemistry Letters 7:1007-1012 (1997), hereby incorporated by reference, particularly for disclosed structures and derivatives thereof.

Other calcineurin inhibitors include 4-(fluoromethyl)phenyl phosphate (FMPP), Born et al., JBC, 270:25651-5 (1995), cantharidin analogues, Baba et al., Bioorganic & Medicinal Chemistry, 13:5164-70 (2005), Baba et al., JACS 125:9740-9 (2003).

Suitable targeting moieties for calcinerin also include a variety of alkylphophonic acid derivatives containing an additional thiol or carboxylate group as inhibitors as alkaline phosphatase and purple acid phosphatase. Myers J K et al., Motifs for metallophosphatase inhibition. Journal of American Chemical Society, 199:3163-3164 (1997), hereby incorporated by reference, particularly for disclosed structures and derivatives thereof.

Another group of calcineurin inhibitors find use as targeting moieties in the present invention are peptide inhibitors of calcineurin. One such peptide is 25-residue peptide based on the sequence of the autoinhibitory domain of calcineurin A subunit, which is a relatively potent inhibitor of calcineurin phosphatase activity. Hashimoto, et al., J. Biol. Chem., 265: 1924-1927 (1990), hereby incorporated by reference, particularly for disclosed structures and derivatives thereof.

(SEQ ID NO: XXX)

I-T-S-F-E-E-A-K-G-L-D-R-I-N-E-R-M-P-P-R-R-D-A-M-P

Another sixteen amino acids peptide (VIVT) was selected from a combinatory peptide library based on the calcineurin docking motif of NF-AT. This peptide selectively interfere with calcineurin-NF-AT interaction without disrupting calcineurin phosphatase activity. Aramburu J. et al., Affinity-driven peptide selection of an NFAT inhibitor more selective than cyclosporine A, Science 285:2129-2133 (1999), hereby incorporated by reference, particularly for disclosed structures and derivatives thereof.

| | |
|---|---|
| M-A-G-P-H-P-V-I-V-I-T-G-P-H-E-E | (SEQ ID: XXXX) |

Another suitable targeting moiety PD 144795 is a benzothiophene derivative that has been shown to have dose-dependent inhibition of calcineurin. Gualberto et al., J Biol Chem 273:7088-7093 (1998), hereby incorporated by reference, particularly for disclosed structures and derivatives thereof.

Also found to be calcineurin inhibitors are proteins, such as calcipressin 1, or Down Syndrome Critical Region 1 (DSCR1), Chan et al., PNAS 102:13075-80 (2005),

The Figures depict a number of clinically tested inhibitors of calcineurin, which are suitable for use as targeting moieties in the present invention, that shows the structure of these known inhibitors along with possible sites of attachment of the linkers and metal binding moieties, as well as possible derivatives.

In addition to these targeting moieties, other known targeting moieties, identified by the screens outlined below or shown to bind to calcineurin can be used. Thus, suitable targeting moieties include, but are not limited to, small organic molecules including known drugs and drug candidates, polysaccharides, fatty acids, vaccines, polypeptides, proteins (including peptides, as described herein), nucleic acids, carbohydrates, lipids, hormones including proteinaceous and steroid hormones, growth factors, receptor ligands, antigens, antibodies and enzymes, (as outlined below, “candidate agents” are included) etc.

Calcineurin activity can be measured using established methods. Calcineurin is a phosphatases and its activity can be measured using different substrate. The substrate could be a protein or a peptide. One example is a portion of the RII subunit of cAMP-dependant protein kinase (PKA) that was phosphorylated. See Fruman et al., Measurement of calcineurin phosphatase activity in cell extracts, Methods: a companion to methods in enzymology 9:146-154 (1996), herein incorporated by reference. The substrate could be small molecule. Calcineurin is able to hydrolyse p-nitrophenol phosphate (pNPP), a chromogenic small molecule compound that has been extensively used in calcineurin assays. The small pNPP molecule binds directly to the active site of calcineurin, and is hydrolyzed into p-nitrophenol (PNP), which can be readily monitored using spectrophotometer. See Sagoo, et al., Competitive inhibition of calcineurin activity by its autoinhibitory domain, Biochem. J. 320:879-884 (1996), herein incorporated by reference.

### Metallo-Beta-Lactamase

One of the most important mechanisms of microbial resistance to β-lactam antibiotics is hydrolysis by β-lactamases (EC 3.5.2.6). Since carbapenems have a broader antimicrobial spectrum than do other β-lactam antibiotics and are not hydrolyzed by many clinically relevant serine β-lactamases, the medical use of carbapenems would be expected to increase. However, there are several carbapenem-hydrolyzing β-lactamases that preferentially hydrolyze carbapenems in addition to penicillins and cephalosporins. The class B MBL, which have zinc atoms at the active site, are a group of such carbapenem-hydrolyzing enzymes and are minimally inhibited by β-lactamase inhibitors such as tazobactam. Besides, widely used serine β-lactamase inhibitors behave as substrates of class B β-lactamases. Nagano et al., Antimicrob Agents Chemother. 43:2497-503 (1999). There are no clinically available inhibitors for MBL. The hydrolysis of cephalosporin beta-lactam antibiotics generates dihydrothiazines which subsequently undergo isomerization at C6 by C—S bond cleavage and through the intermediacy of a thiol. These thiols can be trapped by the beta-lactamase from Bacillus cereus, causing inhibition of the enzyme. NMR studies have identified the structure of the thiols causing inhibition and also show that the thiol binds to the zinc ion, which in turn perturbs the metal-bound histidines. Inhibition is slowly removed as the thiol becomes oxidized or undergoes further degradation. The thiol intermediate generated from cephalothin is a slow binding inhibitor. Badarau et al., Biochemistry 44:8578-89 (2005).

The increase in antibiotic resistance among gram-negative bacteria presents a daunting challenge for the clinical care, and MBL plays an important role in the resistance mechanism of gram-negative bacteria. For general review, see Wash et al., Clinical Microbiology Review, 19:306-25 (2005), herein expressly incorporated by references in its entirety.

The structures of several MBLs have been solved by x-ray diffraction and reveal two potential zinc ion binding sites at the active site. The zinc ligands are not fully conserved between the different subclasses of MBL. In the subclass B1 enzymes, such as the B. cereus enzyme BclI, the zinc in site 1 is coordinated by the imidazole rings of three histidine residues and a solvent molecule. In site 2, the metal is coordinated by a histidine, an aspartic acid, a cysteine, and one or two solvent molecules. The two metal ions are relatively close to each other, but the apparent distance between them ranges from 3.4 to 4.4 Å in different structures of the BclI and CcrA (Bacteroides fragilis) enzymes. Several structures of the CcrA enzyme show a bridging ligand between the two metals, suggested to be an hydroxide ion; however, a bridging solvent molecule is not universally present in structures of this enzyme. In a structure of BclI containing two zinc ions determined at pH 7.5, a similar bridging solvent molecule is seen, but in structures of this enzyme at lower pH, this solvent molecule is much more closely associated to the zinc in site 1 than to that in site 2. The second solvent molecule at site 2 is carbonate or water but is missing in one structure (as well as in structures with inhibitors bound. The coordination of the metal ions is thus quite variable, perhaps contributing to some of the observed differences in substrate profiles and zinc affinities among MBLs. The bridging hydroxide ion or water molecule has been proposed to be the nucleophile responsible for beta-lactam hydrolysis, but the precise role of the two metals in catalysis remains unclear; mechanisms have been proposed in which only site I plays a direct role in catalysis or in which the two zinc ions are both involved as a binuclear center, The BclI enzyme is active with either one or two zinc ions bound with different kinetic characteristics. Daldmon et al., J. Biol. Chem., 278:29240-51 (2003).

Since all members of MBL show two zinc binding sites in close proximity, the development of inhibitors is still focused on the binuclear enzymes. However, it has been shown that only mononuclear MBL might be physiologically important. These findings pose the question whether strategies to find inhibitors for the binuclear enzymes are the only ones being adequate. In order for these inhibitors to be pharmaceutically relevant, one needs to ensure that the inhibition constants are low for all MBLs from pathogenic bacteria even at low zinc abundance. Under such conditions, the mononuclear enzymes are the dominating form. Heinz et al., J. Biol. Chem., 278:20659-66 (2003).

Several classes of MBL inhibitors have been reported, including phenazines, thiols, amino acid-derived hydroxamates. Walter et al., Bioorg. Chem. 27:35-40 (1999), and d- and l-captopril. Although the inhibitors described above have been reported to have good activity against a specific MBL, only certain thiols (e.g. SB 264218) exhibit broad spectrum inhibition of MBLs. Toney et al., J. Biol Chem. 276:31913-18 (2001); Heinz et al., J. Biol. Chem., 278:20659-66 (2003).

Also reported inhibitors of these enzymes are, two esters of benzyloxycarbonylmethyl-6-aminopenicillanic acid, Van Hove et al., Tetrahedron Lett. 36:9313-9316 (1995), a group of α-amido-trifluoromethyl alcohols and ketones, Walter et al, Tetrahedron 53:7275-7290 (1997), Walter et al., Bioinorg. Med. Chem. Lett. 6:2455-2458 (1996), a series of thiol ester derivatives of mercaptoacetic and mercaptophenylacetic acids, Greenlee et., Bioinorg. Med. Chem. Lett: 9:2549-2554 (1999), Hammond et al., FEMS Microbiol. Lett. 179:289-296 (1999), Payne et al., FEMS Microbial. Left. 157:171-175 (1997), Payne et al., Antimicrob. Agents Chemother. 41:135-140 (1997), and derivatives of β-methylcarbapenem (11). More recently, derivatives of cysteinyl peptides have also been tested, Navarro et al., Antimicrob Agents Chemother, 48:1058-1060 (2004).

Biphenyl tetrazoles (BPTs) are a structural class of potent competitive inhibitors of MBL identified through screening and predicted using molecular modeling of the enzyme structure. The tetrazole moiety of the inhibitor interacts directly with one of the two zinc atoms in the active site, replacing a metal-bound water molecule. Toney et al., Chem. Biol. 5, 185-196 (1998); Toney et al., Bioorg. Med. Chem. Lett. 9, 2741-2746 (1999).

Other MLB inhibitors include penamaldic derivatives of penicillins. Navarro et al., Antimicrob Agents Chemother, 48:1058-1060 (2004).

Bulgecin A, a sulphonated N-acetyl-D-glucosamine unit linked to a 4-hydroxy-5-hydroxymethylproline ring by a b-glycosidic linkage, is a novel type of inhibitor for binuclear metallo-b-lactamases. Simm et al., Biochem. J. 387:585-590 (2005).

The IMP-1 gene encoding an MBL has been identified on a plasmid and in Japan has transferred among clinical isolates such as Pseudomonas aeruginosa, Klebsiella pneumoniae, Serratia marcescens, and other members of the Enterobacteriaceae. In addition, carbapenem-resistant clinical isolates expressing MBLs related to IMP-1 have been identified Singapore, Italy, and Hong Kong. Such reports of plasmid-borne imipenem resistance highlight the need for inhibitors of IMP-1 that can restore the activity of carbapenems in resistant bacteria. A series of 2,3-(S,S)-disubstituted succinic acids have been identified as potent inhibitors of IMP-1. Toney et al., J. Biol Chem. 276:31913-18 (2001. There are also three types of 1β-methylcarbapenems having benzothienylthio, dithiocarbamate, or pyrrolidinylthio moieties at the C-2 position showed good inhibitory activity against IMP-1 metallo-beta-lactamase, a MBL. Nagano et al., Antimicrob Agents Chemother. 43:2497-503 (1999).

Thiomandelic acid is a simple, broad spectrum, and reasonably potent inhibitor of MBLs. NMR data suggest thiomandelate binds through its thiolate sulfur to both zinc ions in MBL. Mollard et al., J. Biol. Chem., 276:45015-45023 (2001); Daldmon et al., J. Biol. Chem., 278:29240-51 (2003).

Accordingly, there are a wide variety of suitable targeting moieties. The Figures depict a number of clinically tested inhibitors of beta-lactamase, which are suitable for use as targeting moieties in the present invention, and show the structure of these known inhibitors along with possible sites of attachment of the linkers and metal binding moieties, as well as possible derivatives.

In addition to these targeting moieties, other known targeting moieties, identified by the screens outlined below or shown to bind to MBL can be used. Thus, suitable targeting moieties include, but are not limited to, small organic molecules including known drugs and drug candidates, polysaccharides, fatty acids, vaccines, polypeptides, proteins (including peptides, as described herein), nucleic acids, carbohydrates, lipids, hormones including proteinaceous and steroid hormones, growth factors, receptor ligands, antigens, antibodies and enzymes, (as outlined below, “candidate agents” are included) etc.

MBL activity can be measured using established methods. For example, in one assay, the activity of the metallo-β-lactamase preparation is determined at each step by monitoring the hydrolysis of 100 μM imipenem (Δε=9.04 mM⁻¹ cm⁻at 299 nm) at 30° C. in 10 mM MOPS buffer (pH 7.0) containing 100 μM ZnCl₂. One unit of β-lactamase activity is defined as the amount of enzyme that hydrolyzed 1 μmol of imipenem per min at 30° C. See e.g. Nagano et al., Antimicrob Agents Chemother. 43:2497-503 (1999), herein incorporated by reference. In another assay, the activity was assessed using the chromogenic substrate nitrocefin. See Toney et al., J. Biol Chem. 276:31913-18 (2001), herein incorporated by reference.

### PDE3

A majority of the residues for binding of PDE3A inhibitors are well conserved in all mammalian PDEs (EC 3.1.4.17). However, a few distinct amino acids may be sufficient to differentiate the inhibitors, and unique amino acids in different types of PDE are critical to determine specificity of specific inhibitor. For example, mutation of a nonconserved amino acid T844 to Ala in PDE3A results in a 25-fold increase in Kᵢ for cilostazol but has no effect on the K for milrinone or cGMP or the Kₘ for cAMP in this study. T844 may also plays a decisive role in the selectivity of PDE3A for cilostazol On the other hand, the active sites of PDEs can not only provide various orientations for the inhibitor binding but may also possess subtle different conformations in each PDE family. The conformational difference might thus distinguish and select inhibitors for each family of PDEs, in a key-lock mechanism. Zhang et al., Molecular Pharmacology, 62:514-520 (2002).

Two PDE3 type-selective inhibitors have been used in clinical practice. Cilostazol has antiplatelet, antithrombotic, and vasodilatory effects and has been approved for the treatment of patients with intermittent claudication and for prevention of short- and medium-term vessel closure as well as late restenosis after intracoronary stenting. Milrinone improves the hemodynamic status of heart failure via inotropic/vasodilatory effects attributable to the increase in cardiac intracellular cAMP level. Milrinone is used for the treatment perioperative severe heart failure or marked deterioration of congestive heart failure. Zhang et al., Molecular Pharmacology, 62:514-520 (2002). Other known specific PDE3 inhibitors include olprinone and amrinone, Adachi et al., Eur J Pharmacol. 528:137-43 (2005).

Other inhibitors that are launched are anagrelide, enoximone (1,3-Dihydro-4-methyl-5-(4-(methylthio)benzoyl)-2H-imidazol-2-one), pimobendan and olprione. There are also piroximone and E-5510 (Eisai) in phase-3 trials.

There are a wide variety of suitable targeting moieties. The figures depict a number of inhibitors of PDE3, which are suitable for use as targeting moieties in the present invention, and show the structure of these known inhibitors along with possible sites of attachment of the linkers and metal binding moieties, as well as possible derivatives.

In addition to these targeting moieties, other known targeting moieties, identified by the screens outlined below or shown to bind to PDE3 can be used. Thus, suitable targeting moieties include, but are not limited to, small organic molecules including known drugs and drug candidates, polysaccharides, fatty acids, vaccines, polypeptides, proteins (including peptides, as described herein), nucleic acids, carbohydrates, lipids, hormones including proteinaceous and steroid hormones, growth factors, receptor ligands, antigens, antibodies and enzymes, (as outlined below, “candidate agents” are included) etc.

PDE3 activity can be measured using established methods. For example, see Thompson et al., Assay of cyclic nucleotide phosphodiesterase and resolution of multiple molecular forms of the enzyme. Adv Cyclic Nucleotide Res; 10:69-92 (1979); and Lugnier, Phosphodiesterase Methods and Protocols (Humana Press, 2005), hereby incorporated by reference.

### PDE4

By “PDE4”, “PDE4 protein”, “PDE4 gene” or grammatical equivalents, herein is meant any phosphodiesterase 4 enzyme, including PDE4A, PDE4B, PDE4C and PDE4D.

There are many PDE4 isoforms, of which about twenty are known. Individual isoforms generate by the form PDE4 families (A, B, C and D) are each characterized by unique N-terminal regions. These families play major role in conferring isoform-specific targeting to distinct intracellular sites and signaling complexes. Houslay et al., Biochem. J. 370:1-18 (2003), expressly incorporated herein by reference.

In a preferred embodiment, the PDE4 proteins are from vertebrates and more preferably from mammals, including rodents (rats, mice, hamsters, guinea pigs, etc.), primates, farm animals (including sheep, goat, pigs, cows, horses, etc) and in a preferred embodiment, from humans. However, PDE4 proteins from other organisms may also be used

The sequences of different PDE4 genes, both DNA and protein sequences, are readily available through a variety of resources, such as the Entrez Nucleotides database (a collection of sequences from several sources, including GenBank, RefSeq, and PDB) and Entrez Protein Database (compiled from a variety of sources, including SwissProt, PIR, PRF, PDB, and translations from annotated coding regions in GenBank and RefSeq), both are maintained by the National Center for Biotechnology Information (NCBI) of the National Institutes of Health of the United States, all of which are herein expressly incorporated by reference. Hereinafter the accession numbers referred are the accession number used by the NCBI databases.

There may be multiple entries for the genes encoding each PDE4 enzymes. This is because there are many splicing variants (or transcription variants, isoforms, splicing isoforms) for each gene. In addition, the same gene may have been cloned and reported several times. Any of the sequences in these entries could be used in the present invention. Here are some exemplary entries of human PDE4 genes: PDE4A (Accession Nos. BC038234, BC019864, NM_{—}006202), PDE4B (Accession Nos. BC105040, NM_{—}001037341, NM_{—}002600), PDE4C (BC109067, U88712, U66347), PDE4D(NM_{—}006203, AY245867, BT007398), herein all incorporated by reference

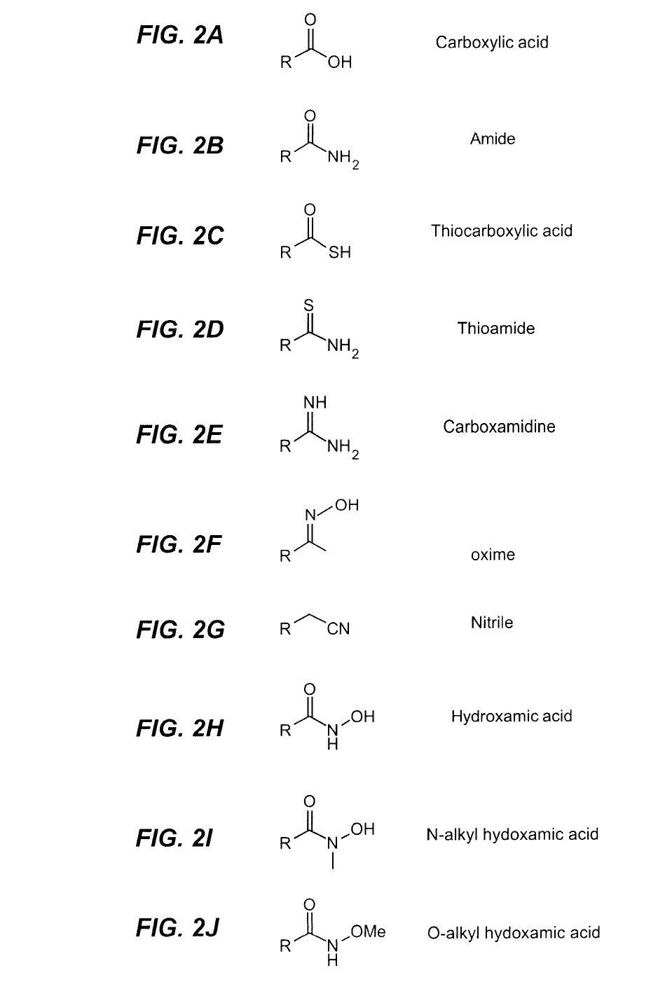

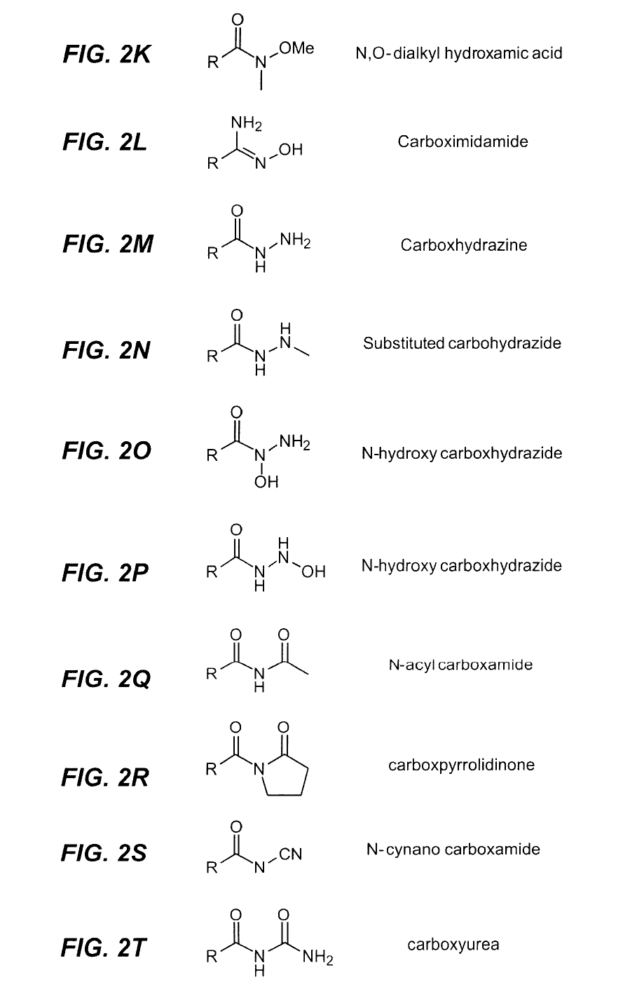

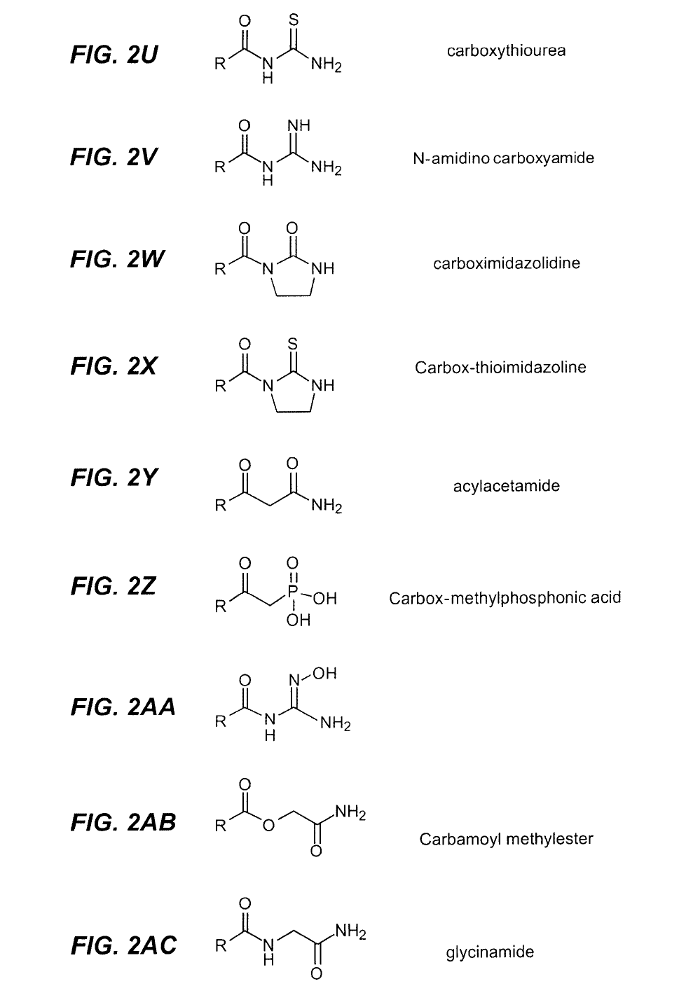

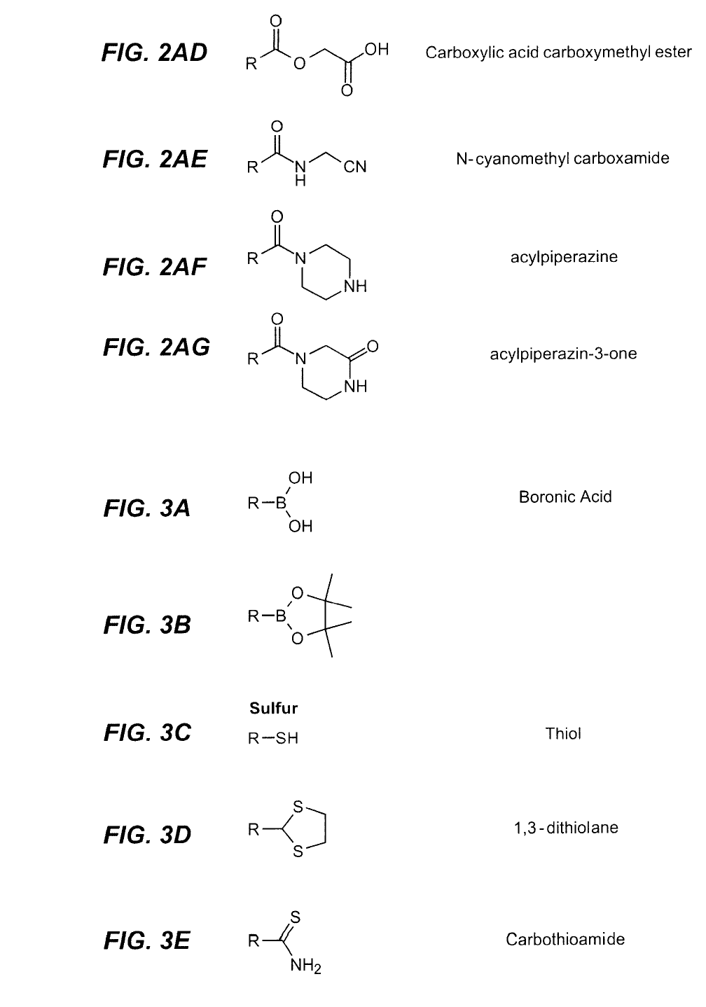

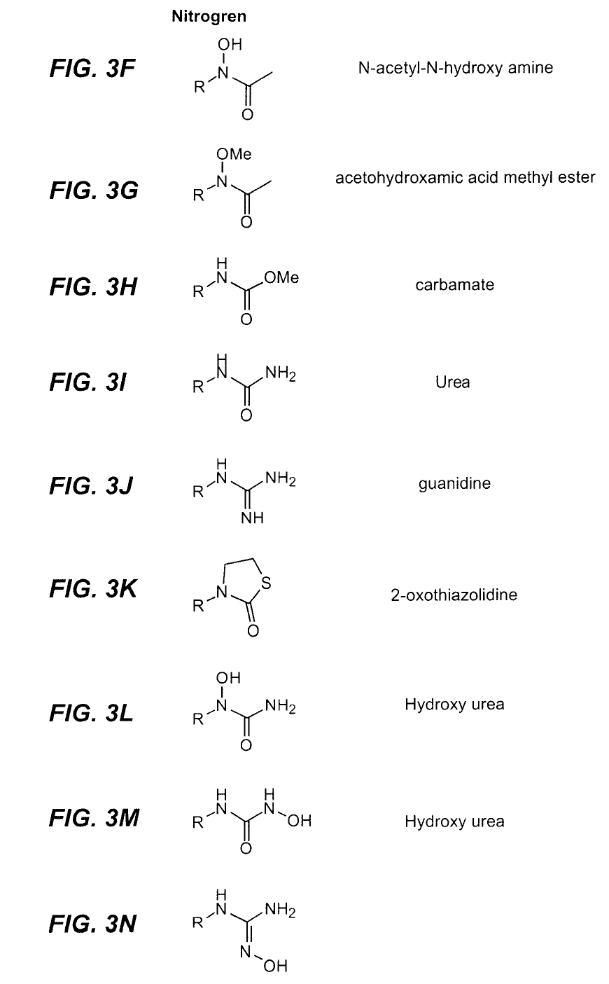

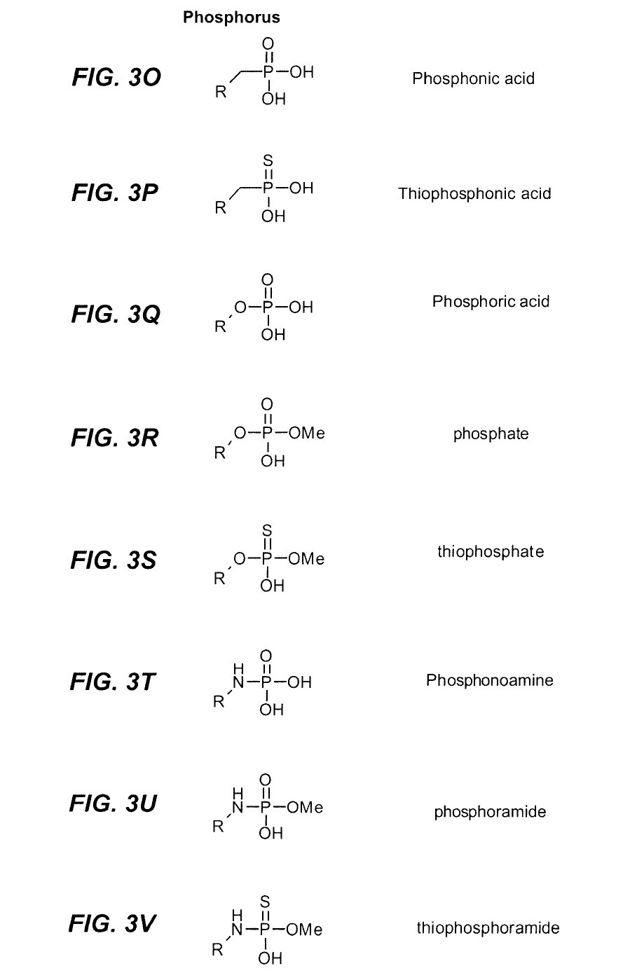

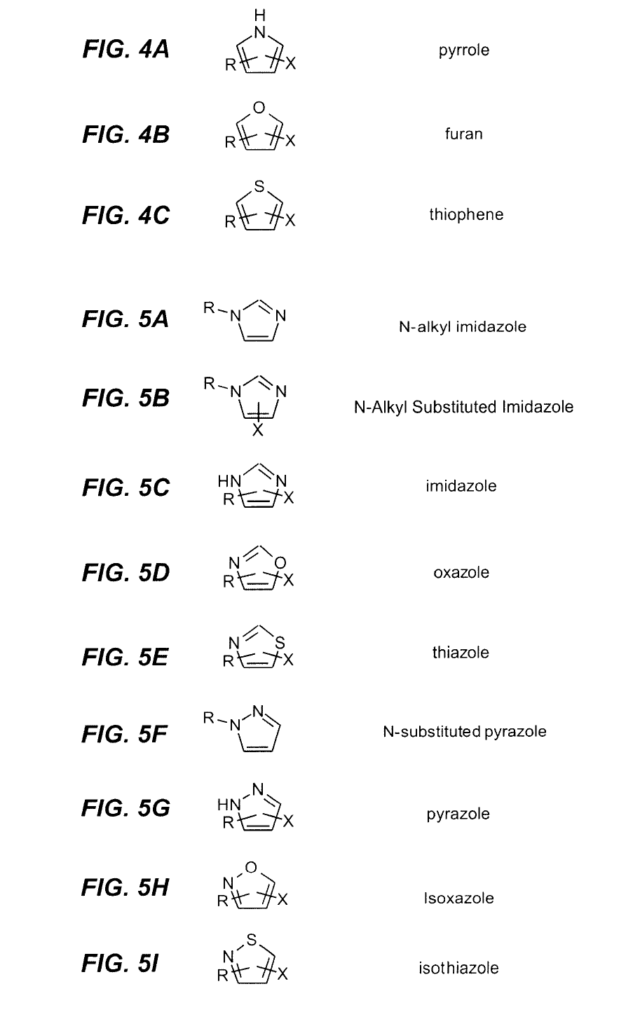

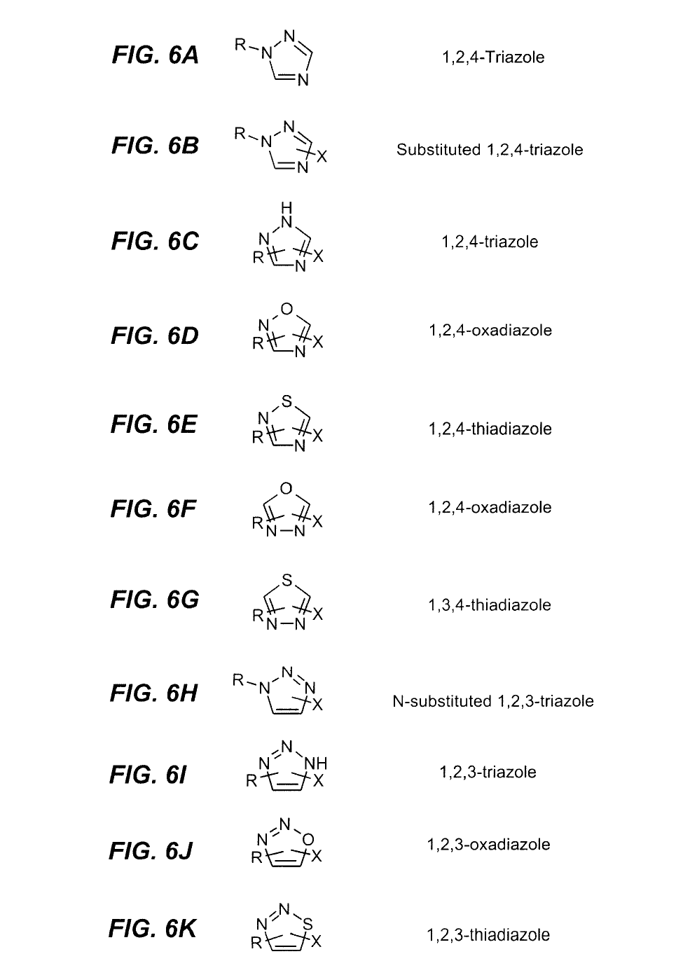

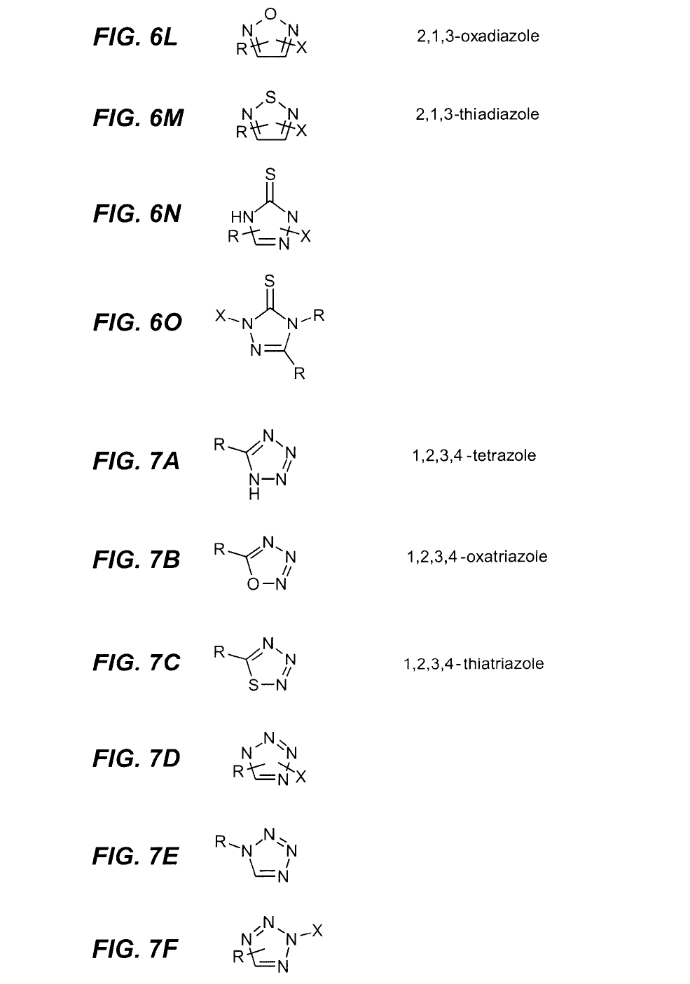

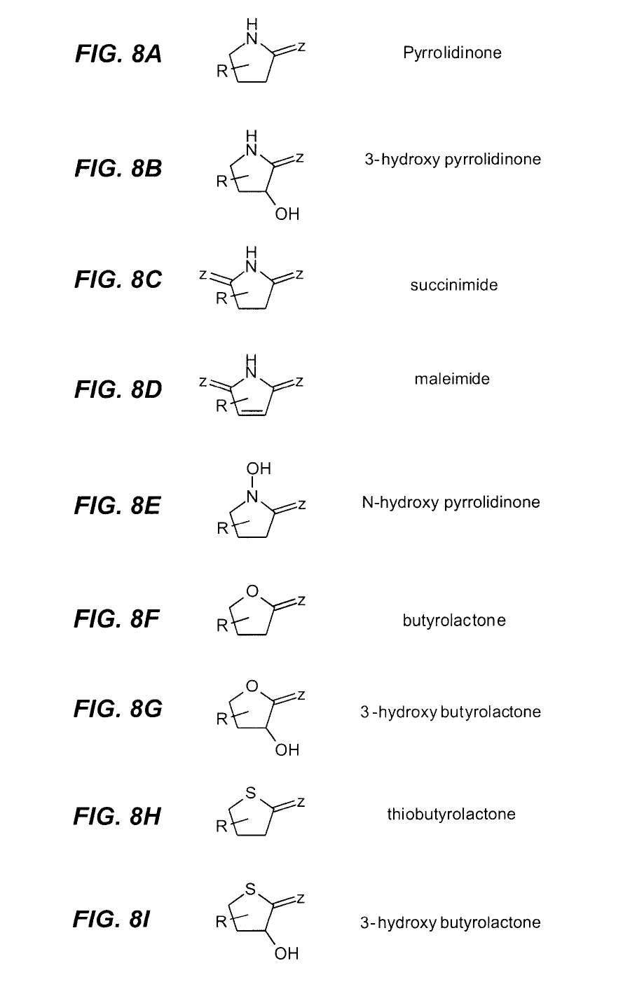

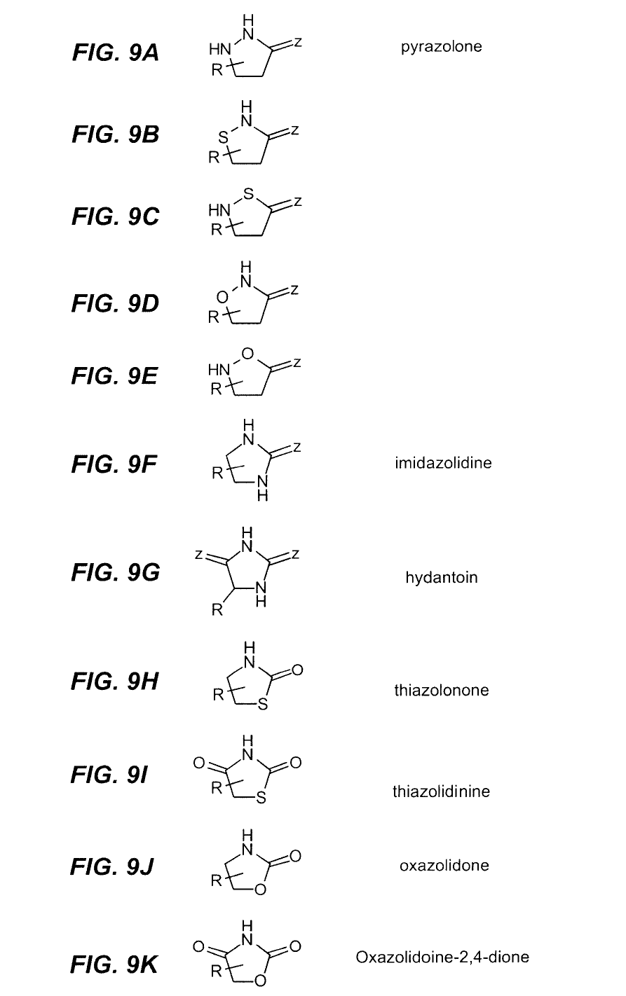

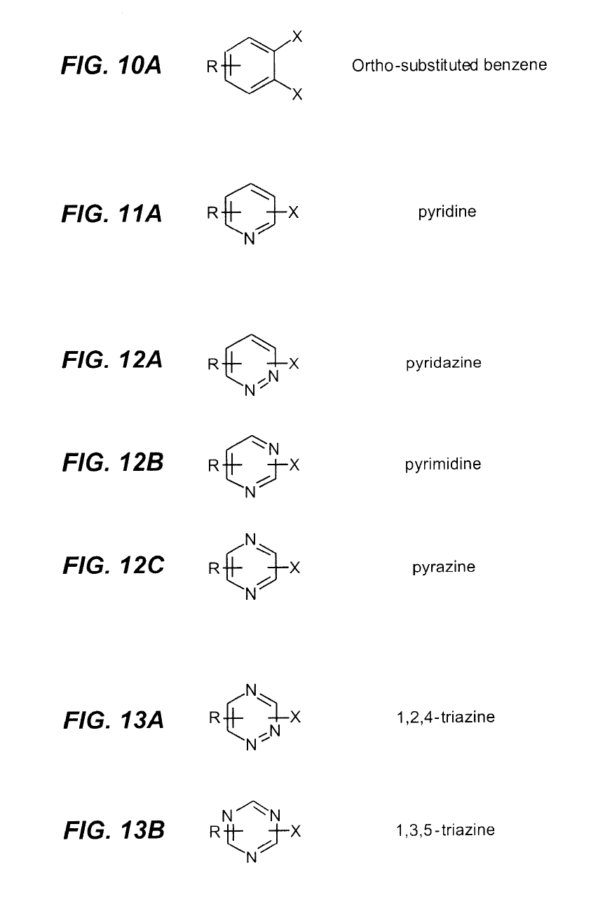

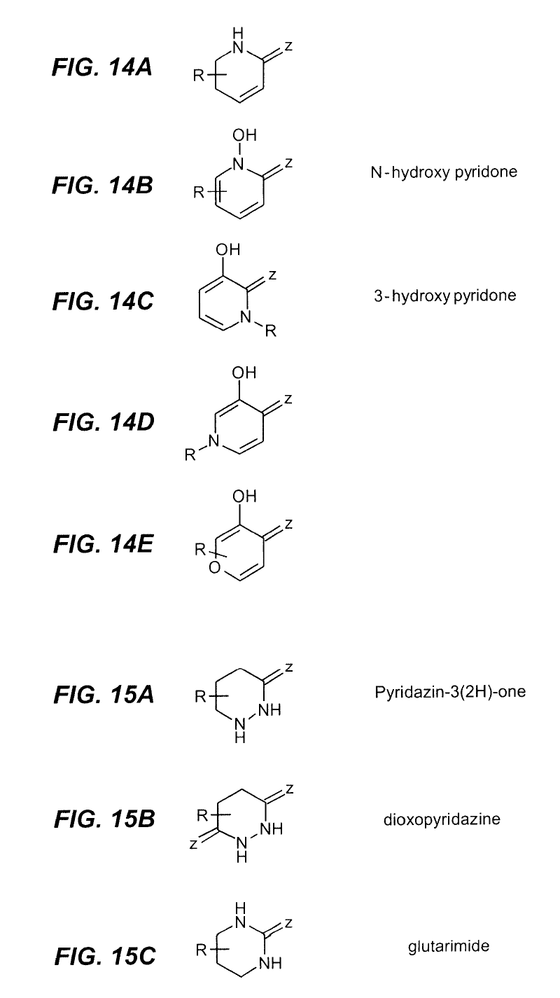

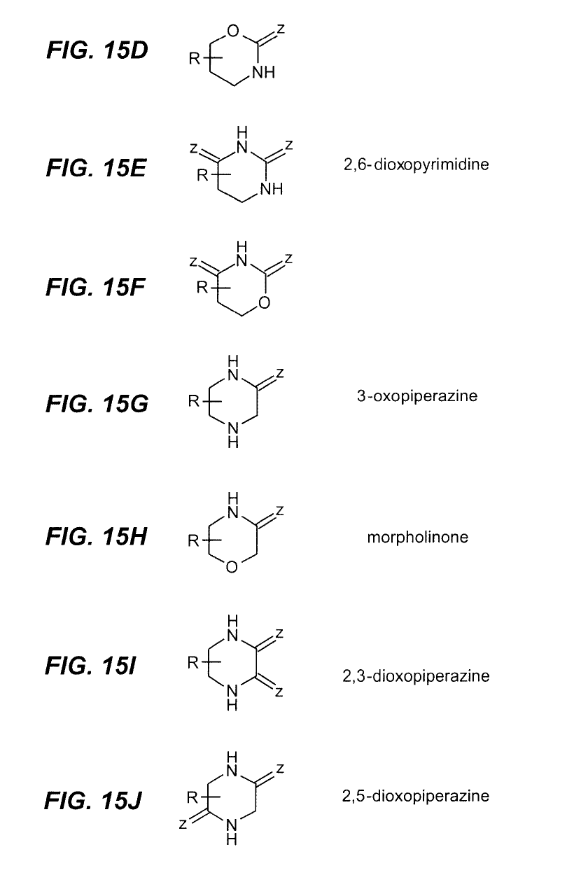

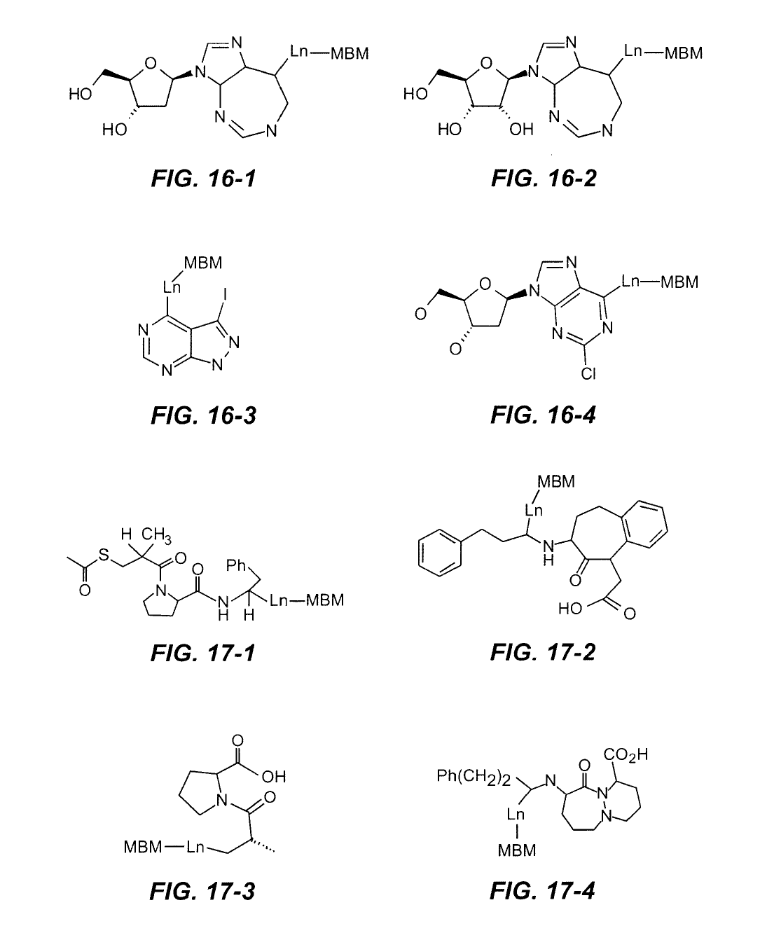

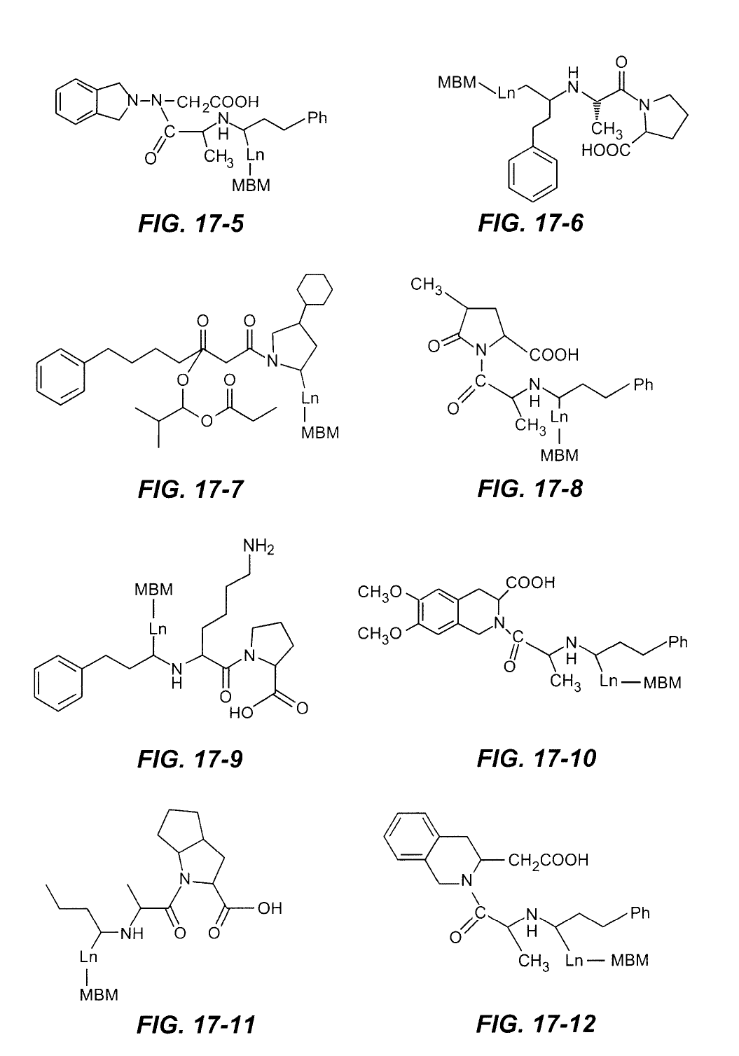

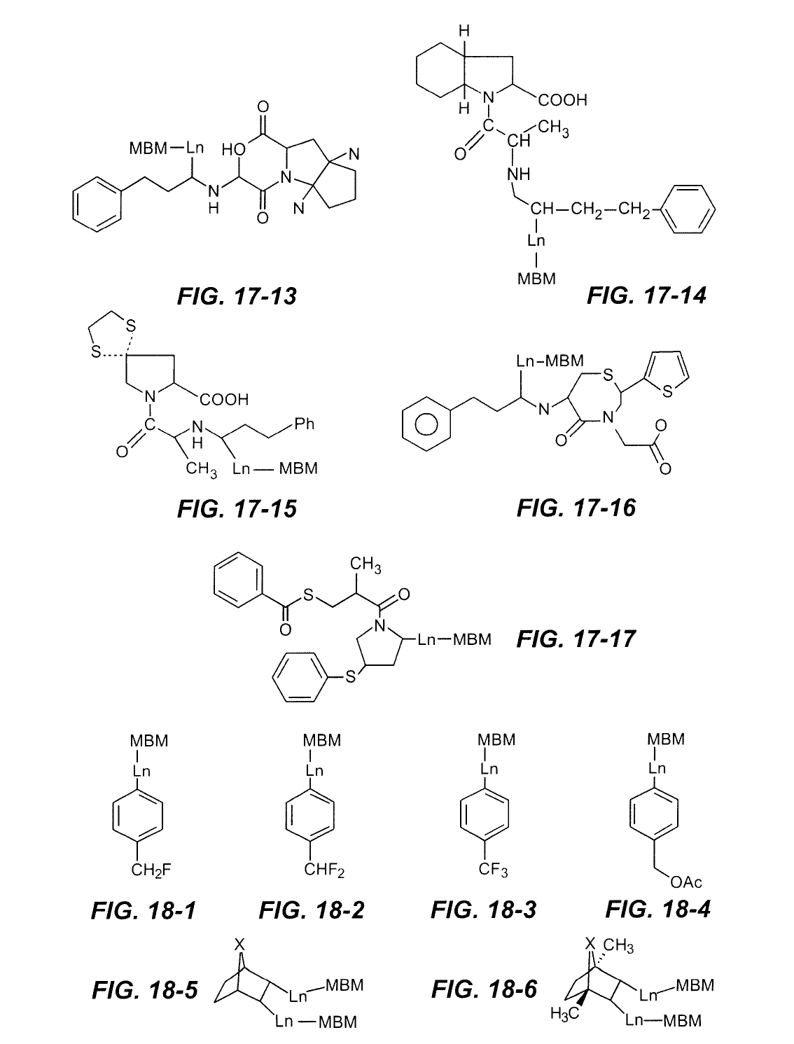

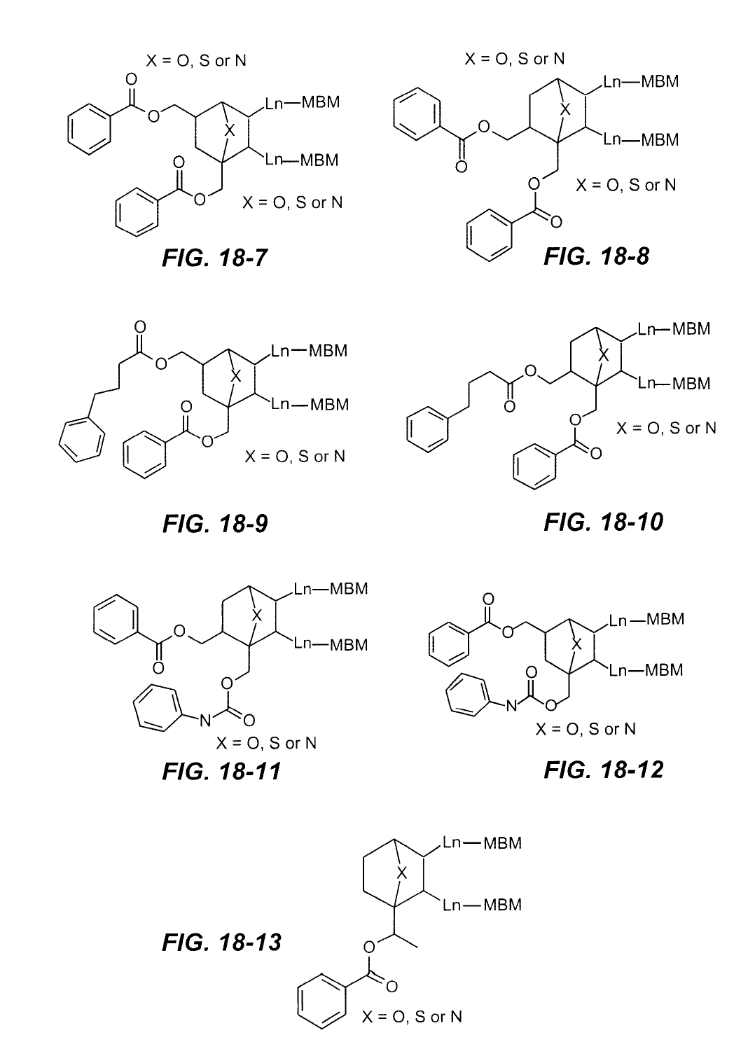

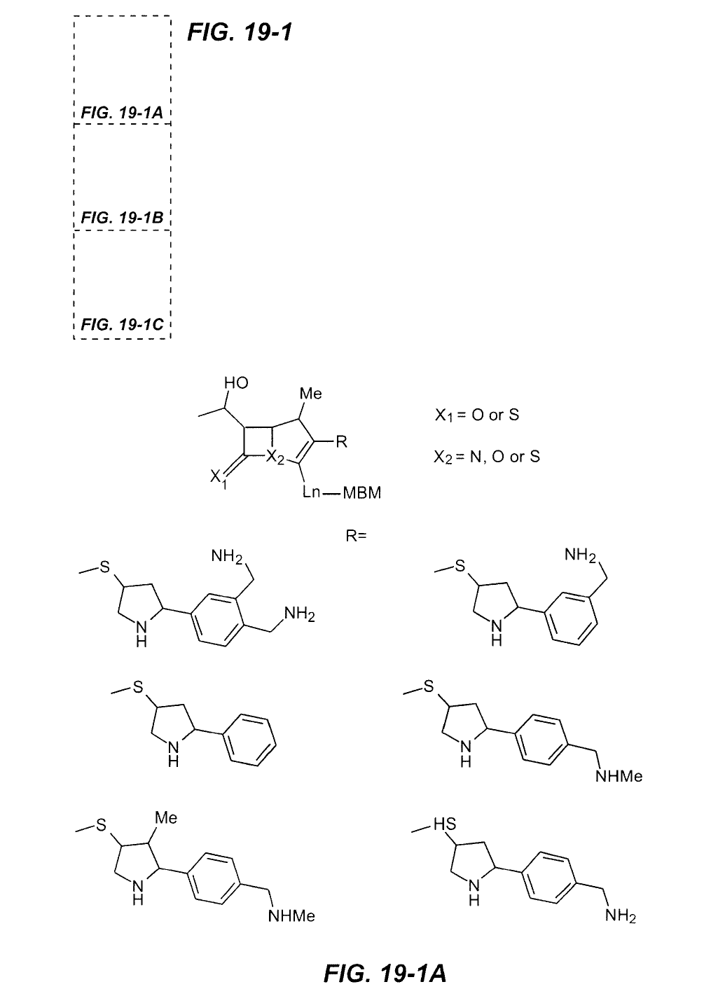

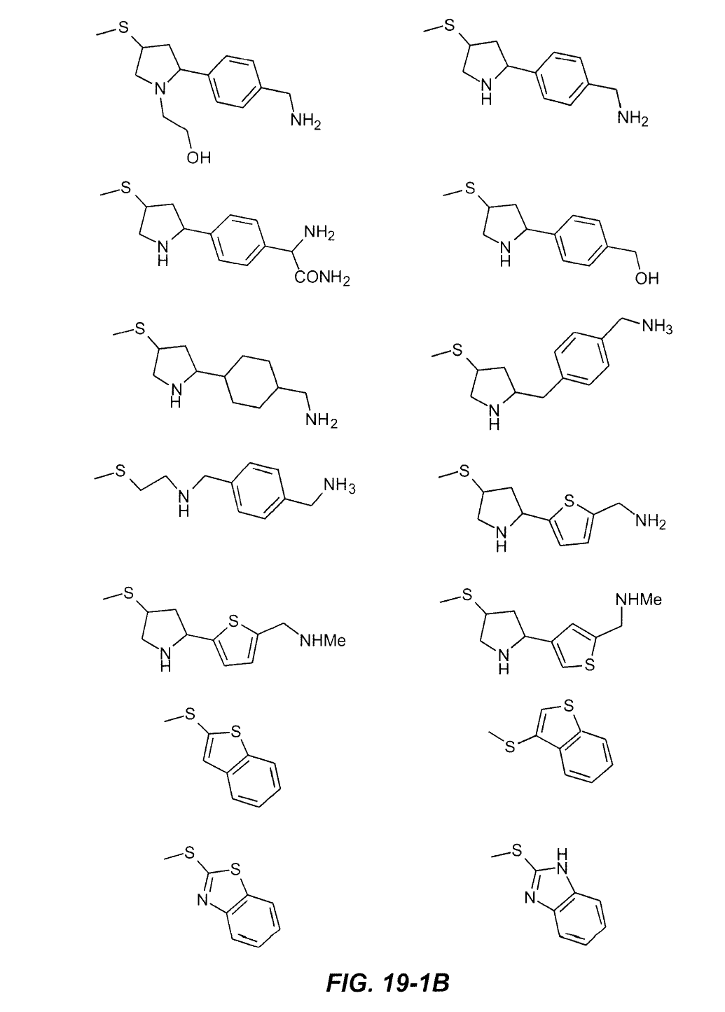

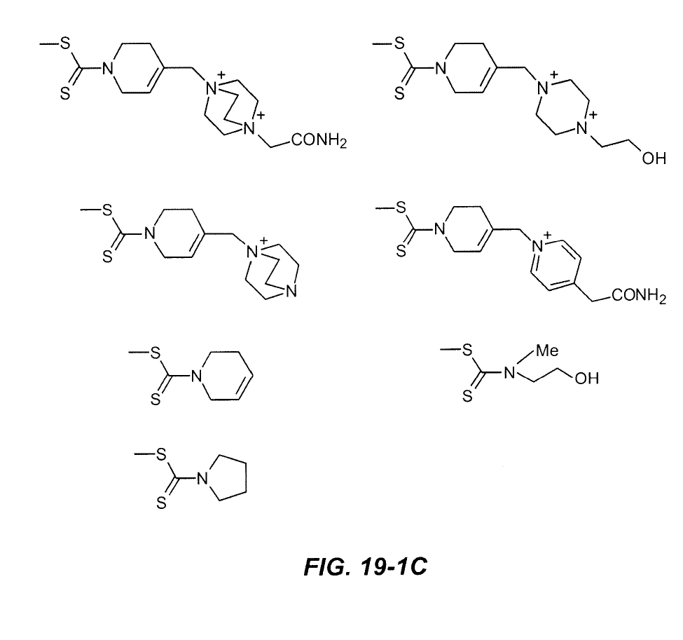

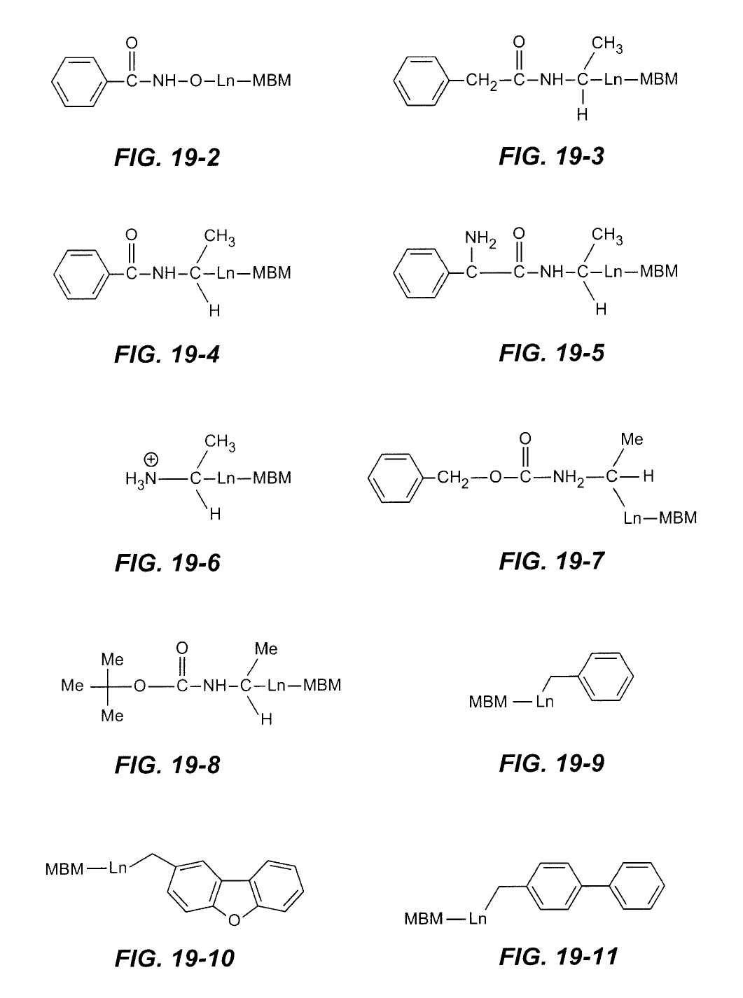

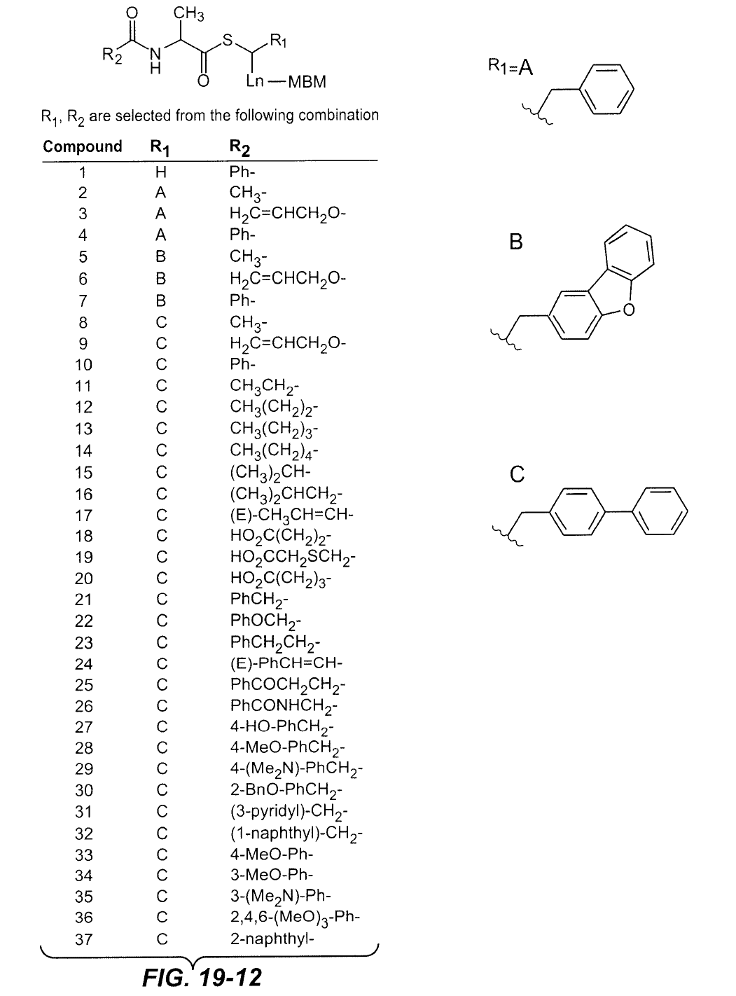

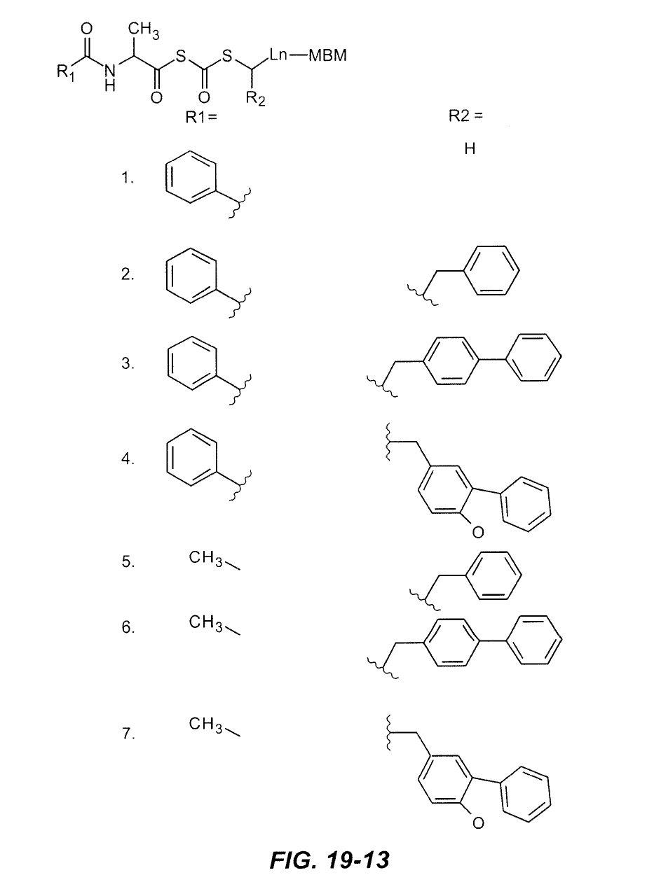

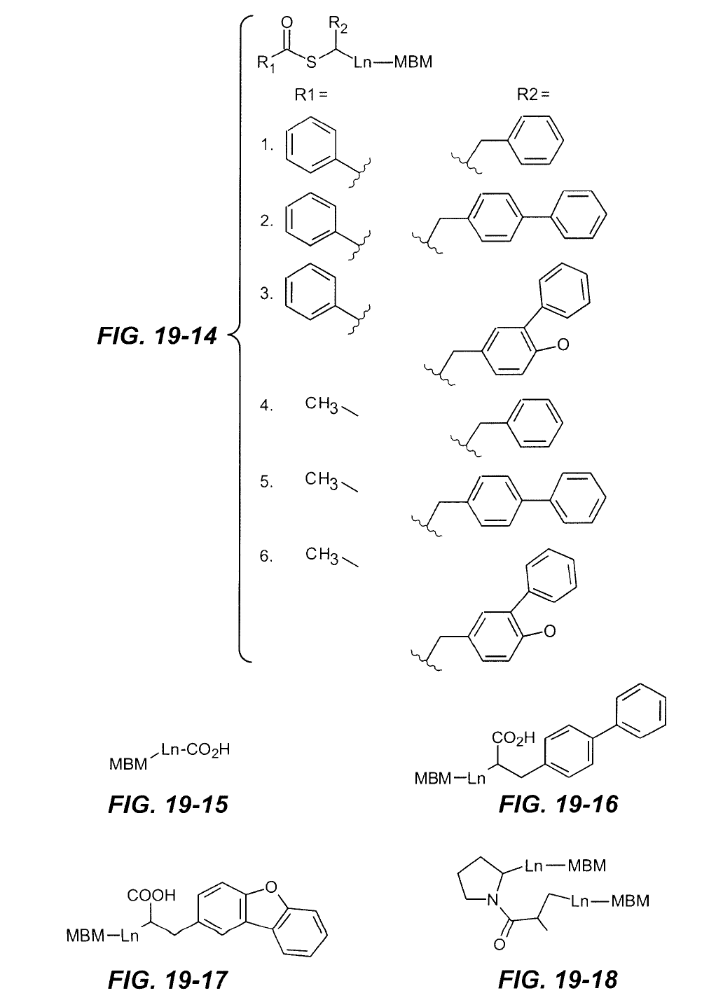

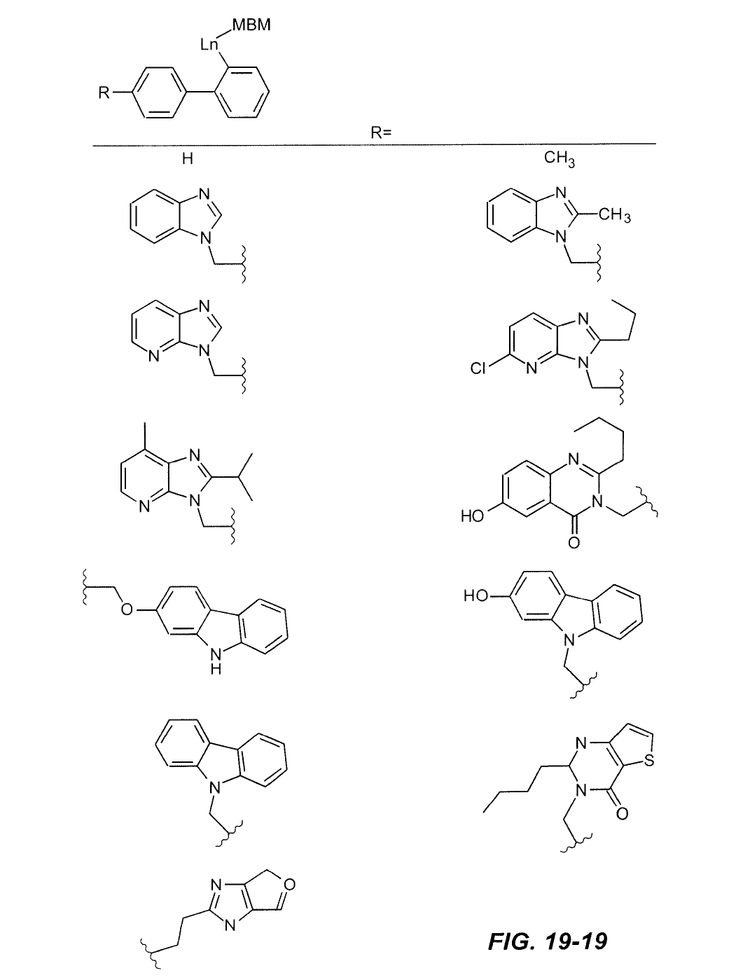

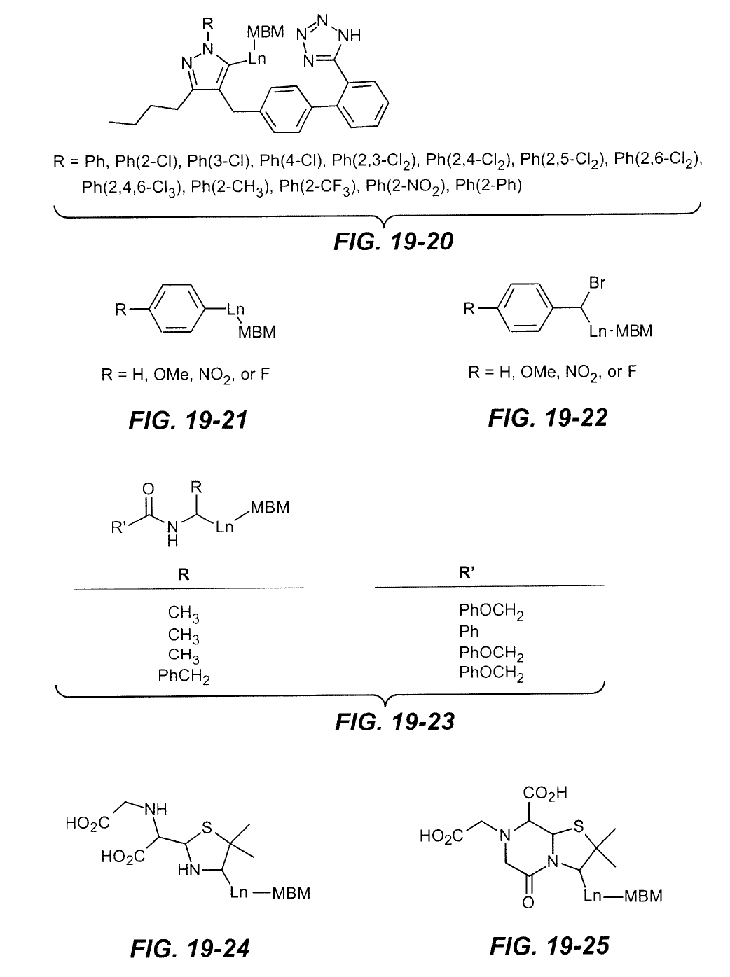

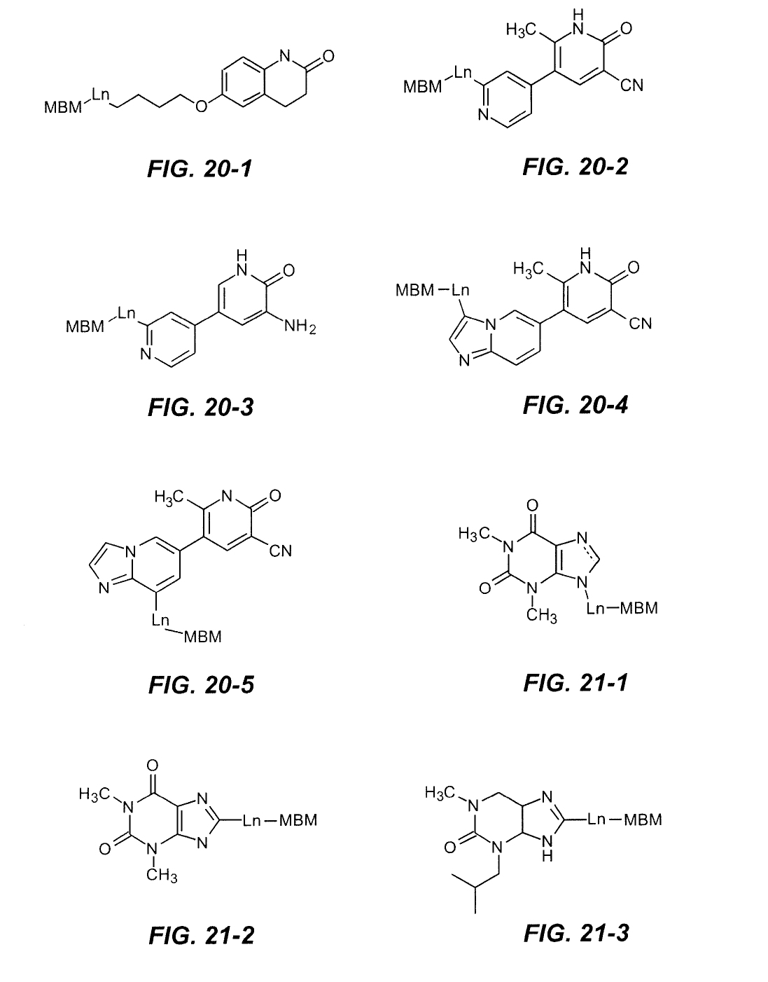

As is known for PDE4, there are several areas suitable for targeting moiety binding. In general, the catalytic domain can be divided into three functions groups that are responsible for nucleotide recognition (N321, Y329, P372 and Q369, using the numbering of PDE4D2 (Accession No. AAC00070), the hydrophobic clamp (I336 and F372), and hydrolysis (D318, H164, D201 and H160); see Houslay article, FIG. 2. The active site is divided into three subpockets, a pocket containing the purine-selective glutamine and the hydrophobic clamp (Q), a solvent filled side pocket (S) and the metal binding pocket which contains both metal ions (M), with the Q subpocket being further divided into Q1, Q2 and Qp regions. In some embodiments, targeting moieties are directed to the O and S pockets, to bring the metal binding moieties in the vicinity of the M pocket. In some embodiments, the targeting moiety will have a planar ring structure that is held in the active site by a pair of hydrophobic resides forming a “hydrophobic clamp”, and there are H-bond interactions with the invariant glutamine residue that is essential for nucleotide selectivity.

There are a wide variety of suitable targeting moieties. The figures depict a number of inhibitors of PDE4, many of which have had components removed as outlined herein, which are suitable for use as targeting moieties in the present invention. The figures show the structure of these known inhibitors along with possible sites of attachment of the linkers and metal binding moieties, as well as possible derivatives.

Additional PDE4 inhibitors have been described, and many specifically depicted, in U.S. Pat. Nos. 6,569,890; 6,909,002; 5,665,754; 6,998,416; 6,747,035; 6,70,666; 6,998,416; 5,665,754; 6,362,213; 6,569,890; 6,589,951; 6,677,351; 6,740,066; 6,699,890; 6,569,885 6,545,158; 6,525,055; 6,498,160; 6,492,360; 6,486,186; 6,458,787; 6,455,562; 6,444,671; 6,423,710; 6,372,777; 6,365,606; 6,358,973; 6,329,370; 6,262,040; 6,294,541; 6,294,561; 6,297,248; 6,303,789; 6,239,130; 6,153,630; 6,103,749; 6,075,016; 6,054,475; 6,043,263; 5,922,751; 5,852,190; 4,921,862; all of which are incorporated herein specifically for the compounds described herein, as well as derivatives such as described herein (e.g. the removal of carboxylic groups and the addition of an optional linker and a metal binding moiety, etc.).

Other known PDE inhibitors are disclosed in WO 2006/050236, WO 2006/050054 and WO 2006/050053, herein all incorporated by references in their entireties. Specially, the structures depicted in formula (1) to (16) of WO 2006/050236, formula (1) to (20) of WO 2006/050054 and formula (1) to (77) of WO 2006/050053 are incorporated by reference, wherein the MBM can be linked to the nitrogen via an optional linker (Ln, wherein n is o or 1) in replacement of the boric acid depicted therein.

In addition to these targeting moieties, other known targeting moieties, identified by the screens outlined below or shown to bind to PDE4 can be used. Thus, suitable targeting moieties include, but are not limited to, small organic molecules including known drugs and drug candidates, polysaccharides, fatty acids, vaccines, polypeptides, proteins (including peptides, as described herein), nucleic acids, carbohydrates, lipids, hormones including proteinaceous and steroid hormones, growth factors, receptor ligands, antigens, antibodies and enzymes, (as outlined below, “candidate agents” are included) etc.

PDE4 activity can be measured using established methods. It is described in more detail below.

### PDE5

Cyclic GMP-binding cGMP-specific phosphodiesterase (PDE5) has been recognised in recent years as an important therapeutic target. It plays a prominent role in cGMP breakdown in lung, platelets, gastrointestinal epithelial cells, Purkinje cells of the cerebellum, and vascular smooth muscle. PDE5 is composed of the conserved C-terminal, zinc containing, catalytic domain, which catalyses the cleavage of cGMP, and an N-terminal regulatory portion, which contains two GAF domain repeats. Each GAF domain contains a cGMP-binding site, one of high affinity and the other of lower affinity. PDE5 activity is regulated through binding of cGMP to the high and low affinity cGMP binding sites followed by phosphorylation, which occurs only when both sites are occupied. PDE5 is found in varying concentrations in a number of tissues including platelets, vascular and visceral smooth muscle, and skeletal muscle. The protein is a key regulator of cGMP levels in the smooth muscle of the erectile corpus cavemosal tissue. The physiological mechanism of erection involves release of nitric oxide (NO) in the corpus cavemosum during sexual stimulation. NO then activates the enzyme guanylate cyclase, which results in increased levels of cGMP, producing smooth muscle relaxation in the corpus cavemosum and allowing in flow of blood. Inhibition of PDE5 inhibits the breakdown of cGMP allowing the levels of cGMP, and hence smooth muscle relaxation, to be maintained. U.S. Patent Application Publication No. 20050202549.

PDE5 is the target of sildenafil (Viagra™), tadalafil (Cialis™), and vardenafil (Levitra™), all of which are in use for treatment of maladies associated with vascular disease. Zoraghi et al., J. Biol. Chem., 280:12051-63 (2005). Other inhibitors that have been launched are dipyridamole, udenafil, and that are in clinical trials include UK-357903 (Pfizer), UK-369003 (Pfizer), avanafil, paragrelil, OSI-461 and E-4021 (Eisai).

Sophoflavescenol, a C-8 prenylated flavonol, has been shown to be a potent inhibitory activity PDE5. Shin et al., Bioorg Med Chem Lett. 12:2313-16 (2002).

Tetracyclic guanines have been shown to be potent and selective inhibitors of the cGMP-hydrolyzing enzymes PDE1 and PDE5. Ahn et al., J Med Chem. 40:2196-210 (1997).

Other examples of PDE5 inhibitors are listed in U.S. Patent Application Publication No. 20040132731, herein expressly incorporated by its entirety.

There are a wide variety of suitable targeting moieties. The Figures depict a number of inhibitors of PDE5, which are suitable for use as targeting moieties in the present invention, and show the structure of these known inhibitors along with possible sites of attachment of the linkers and metal binding moieties, as well as possible derivatives.

In addition to these targeting moieties, other known targeting moieties, identified by the screens outlined below or shown to bind to PDE5 can be used. Thus, suitable targeting moieties include, but are not limited to, small organic molecules including known drugs and drug candidates, polysaccharides, fatty acids, vaccines, polypeptides, proteins (including peptides, as described herein), nucleic acids, carbohydrates, lipids, hormones including proteinaceous and steroid hormones, growth factors, receptor ligands, antigens, antibodies and enzymes, (as outlined below, “candidate agents” are included) etc.

PDE5 activity can be measured using established methods. See e.g. Movesian et al., J Clin Invest., 88: 15-19 (1991); Rybalkin et al., EMBO J. 22: 469-478 (2003); Champion et al., Proc Natl Acad Sci USA. 102: 1661-1666 (2005).

### Renal Dipeptidase

Renal Dipeptidase (RDP) (EC 3.4.13.19, also known as membrane dipeptidase (MDP); dehydropeptidase I (DPH I); dipeptidase; aminodipeptidase; dipeptide hydrolase; dipeptidyl hydrolase; nonspecific dipeptidase; glycosyl-phosphatidylinositol-anchored renal dipeptidase), has been extensively analyzed with respect to its catalytic mechanism and inhibition kinetics by variety of synthetic inhibitors. RDP is unique among the dipeptidases in that it can cleave amine bonds in which the COOH-terminal partner is a D-amino acid. RDP is a zinc-containing hydrolytic enzyme that shows preference for dipeptide substrates with dehydro amino acids at the carboxyl position. Moreover, it can accommodate substrates with both D- or L-amino acids at that position, providing an excellent opportunity for the development of specific probes for its detection in vivo.

α-Aminophosphinic acids, the phosphorous analogues of natural occurring α-aminocarboxylic acids, have received increasing interest in medicine and synthetic organic chemistry. The crystal structure of RDP-cilastatin complex has demonstrated that the dipeptidyl moiety of cilastatin is sandwiched between the negatively charged and positively charged sidewalls. Both ends of the moiety are clamped tightly by hydrophobic interactions. Certain aminophosphinic acid derivatives bind to the active site of RDP similar to dipeptides. Dehydropeptide analogs whose scissile carboxamide has been replaced with a PO(OH)CH₂ group have been found to be potent inhibitors of the zinc protease dehydrodipeptidase 1 (DHP-1 renal dipeptidase, EC 3.4.13.11). α-aminophosphinic acids bearing a hydrophobic side chain have been found to inhibit APN in the 10⁻⁷ molar range. Phosphinate analogs have been reported for inhibition of enzymatic activity of VanX. U.S. Patent Application Publication 20050271586, herein expressly incorporated by its entirety.

RDP exhibits versatile substrate specificity, hydrolyzing not only dipeptides and dehydropeptides but also β-lactam antibiotics of the trans-conformation, such as imipenem. Thienamycin and related carbapenem antibiotics are rapidly hydrolyzed and inactivated in vivo in humans by also commonly referred to as dehydropeptidase. Cilastatin (MK0791; {Z-S-[6-carboxy-6-(2,2-dimethyl-(S)-cyclopropyl)carboxy)-amino-5-hexenyl]-L-cysteine}) was developed as a reversible, competitive inhibitor of RDP (50% inhibitory concentration 50.1 mM) on the basis of the structural similarities between the scissile bonds in imipenem and dehydropeptides. Keynan et al., Antimicrobial Agents And Chemotherapy, 39:1629-1631 (1995).

There are a wide variety of suitable targeting moieties. The figures depict a number of inhibitors of renal dipeptidase, which are suitable for use as targeting moieties in the present invention, and show the structure of these known inhibitors along with possible sites of attachment of the linkers and metal binding moieties, as well as possible derivatives.

In addition to these targeting moieties, other known targeting moieties, identified by the screens outlined below or shown to bind to renal dipeptidase can be used. Thus, suitable targeting moieties include, but are not limited to, small organic molecules including known drugs and drug candidates, polysaccharides, fatty acids, vaccines, polypeptides, proteins (including peptides, as described herein), nucleic acids, carbohydrates, lipids, hormones including proteinaceous and steroid hormones, growth factors, receptor ligands, antigens, antibodies and enzymes, (as outlined below, “candidate agents” are included) etc.

RDP activity can be measured using established methods. See e.g. Keynan et al., Antimicrobial Agent and Chemotherapy, 39:1629-31 (1995).

### Urease

Nickel-dependent urease (urea amidohydrolase, EC 3.5.1.5) have been isolated from various bacteria, fungi, and higher plants. Their primary environmental role is to allow the organism to use external and internally generated urea as nitrogen sources. In plant, urea probably also participates in systematic nitrogen transport pathway and possibly act as toxic defense protein. The best The best characterized bacterial urease is that from Klebsiella aerogenes. The native enzyme has three subunits, α (60.3 kD, UreC), β (11.7 kD, UreB), and γ (11.1 kD, UreA), reportedly associating with (αβ₂γ₂)₂ stoichiometry. It is a tightly associate trimer of (αβγ)-units in a triangular arrangement The two nickel sites are 3.5 A apart. Ni-1 is coordinated by three ligands. Ni-2 is coordinated by five ligands. Jabri et al., Science 268:998-1004 (1995).

One diterpene ester with a myrsinol-type skeleton have been isolated from Euphorbia decipiens has been shown to be an inhibitor of urease enzyme. Ahmad et al., Chem Pharm Bull (Tokyo), 56:719-23.2003. Other inhibitors include acetohydroxamic acid (AHA), phenylphosphorodiamidate (PPDA), N-(n-butyl) thiophosphoric triamide (NBPT), Ludden et al., Journal of Animal Science, 78:181-7 (2000); fluorofamide [N-(diaminophosphinyl)-4-fluorobenzene] (FFA), Pope et al., Digestive Disease Sciences, 43:109-19 (1998); YJA20379, Woo et al., Arch Pharm Res. 21:6-11 (1998); ecabet sodium, Ito et al., Biol Pharm Bull., 18:850-3 (1995), and rabeprazole, Park et al., Biol Pharm Bull. 19:182-7 (1996).

By screening of a highly diverse 25-mer combinatorial library and random 6-mer peptide libraries on solid phase H. pylori urease holoenzyme, two peptides, 24-mer TFLPQPRCSALLRYLSEDGVIVPS and 6-mer YDFYWVV were identified that can bind and inhibit the activity of urease purified from H. pylori. Houimel et al., Eur. J. Biochem. 262, 774-780 (1999).

There are a wide variety of suitable targeting moieties. The figures depict a number of inhibitors of urease, which are suitable for use as targeting moieties in the present invention, and show the structure of these known inhibitors along with possible sites of attachment of the linkers and metal binding moieties, as well as possible derivatives.

In addition to these targeting moieties, other known targeting moieties, identified by the screens outlined below or shown to bind to urease can be used. Thus, suitable targeting moieties include, but are not limited to, small organic molecules including known drugs and drug candidates, polysaccharides, fatty acids, vaccines, polypeptides, proteins (including peptides, as described herein), nucleic acids, carbohydrates, lipids, hormones including proteinaceous and steroid hormones, growth factors, receptor ligands, antigens, antibodies and enzymes, (as outlined below, “candidate agents” are included) etc.

Urease activity can be measured using established methods. See, e.g. Houimel et al., Eur. J. Biochem. 262, 774-780 (1999), and Stingl et al., Infection and Immunity, 69:1178-1180, (2001); Clemens et al., J Bacteriol., 177:5644-5652 (1995), and Breitenbach and Hausinger, Biochem. J. 250:917-920 (1988).

### Linkers

The inhibitors of the present invention also optionally include a linker. That is, in some instances, the targeting moiety is linked directly to the metal binding moieties. Optionally, linkers comprising at least one atom can be used. By “linker” herein is meant at least one atom that provides a covalent linkage between the metal binding moiety and the targeting moiety. Linkers are generally depicted in the figures as “Ln”, with n being either 0 (e.g. no linker is present such that there is a covalent bond between the targeting moiety and the MBM) or 1 (e.g. a linker is present). In some cases, there may be a single linker used, for example when the inhibitor has the general formula MBM-linker-TM or TM-linker-MBM. Alternatively, several linkers could be used; for example, in the case where more than one metal binding moiety or more than one targeting moiety is used: MBM1-linker-MBM2-linker-TM, MBM1-linker-TM-linker-MBM2, etc. When more than one MBM is used, one or more linkers may optionally be used.

The selection of the linker is generally done using well known molecular modeling techniques. In addition; the length and composition of the linker may be important in order to achieve optimal results. For example, many embodiments utilize linkers with high degrees of freedom, such as short straight alkyl chains such as C1-C6 and sometimes C3-C6 that are generally unsubstituted, although smaller substituent groups find use in some embodiments. Similarly, in some cases, more rigid linkers can be used. In general, preferred linker length and composition can be modeled using the crystal structure of the metalloprotease. Preferred linkers include, but are not limited to, alkyl or aryl groups, including substituted alkyl, cycloalkyl, heteroalkyl and cycloheteroalkyl groups, and substituted aryl and heteroaryl groups, as outlined herein. In some embodiments, tinkers comprising aryl groups such as phenyl are not preferred.

In some cases, the metal binding moieties and targeting moieties are covalently attached using well known chemistries. In many cases, both the metal binding moieties and the targeting moiety contains a chemical functional group that is used to add the components of the invention together, as is outlined herein. Thus, in general, the components of the invention are attached through the use of functional groups on each that can then be used for attachment. Preferred functional groups for attachment are amino groups, carboxy groups, oxo groups and thiol groups. These functional groups can then be attached, either directly or indirectly through the use of a linker. Linkers are well known in the art; for example, homo-or hetero-bifunctional linkers as are well known (see 1994 Pierce Chemical Company catalog, technical section on cross-linkers, pages 155-200, incorporated herein by reference). Alternatively, the whole molecule is synthesized in steps, rather than by joining two pieces.

### Inhibitors of the Invention

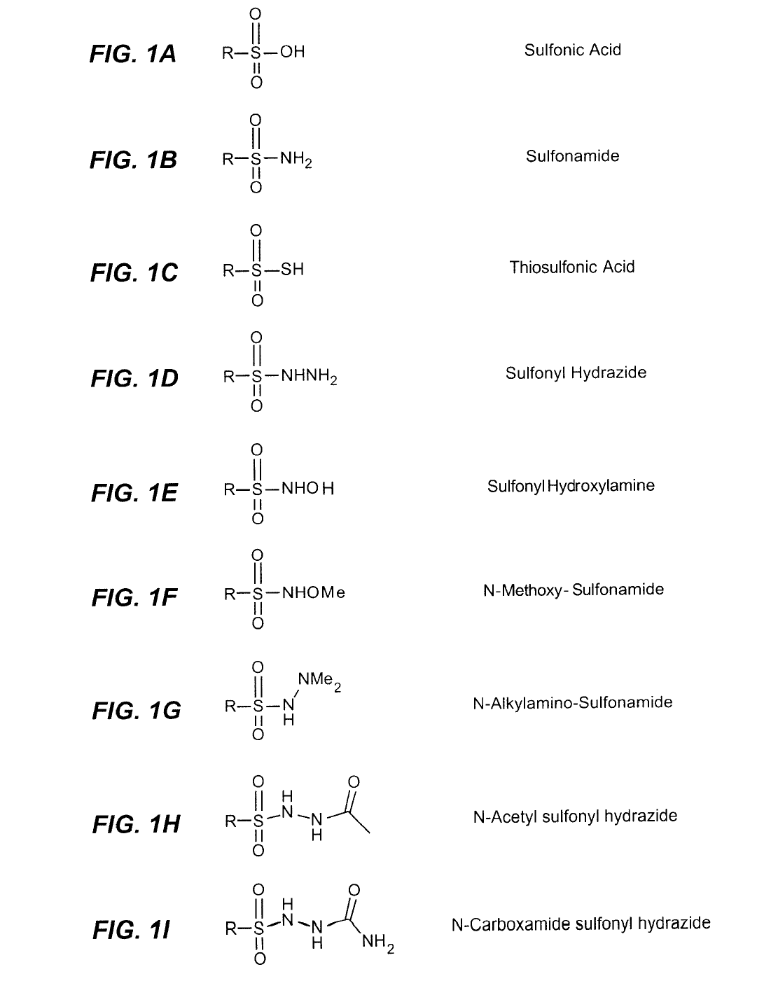

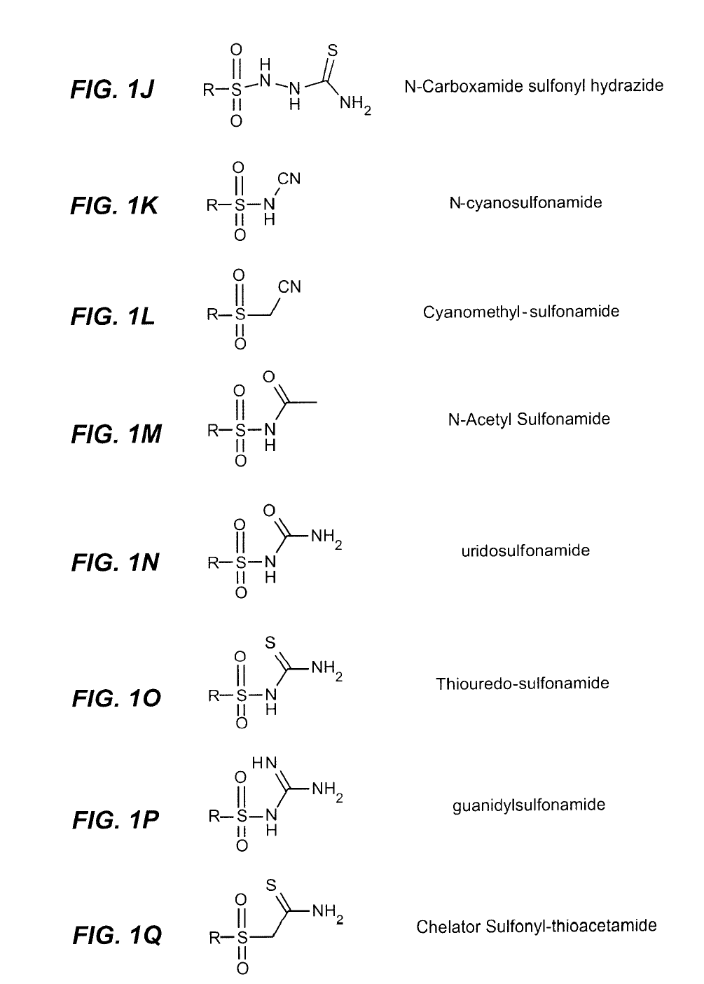

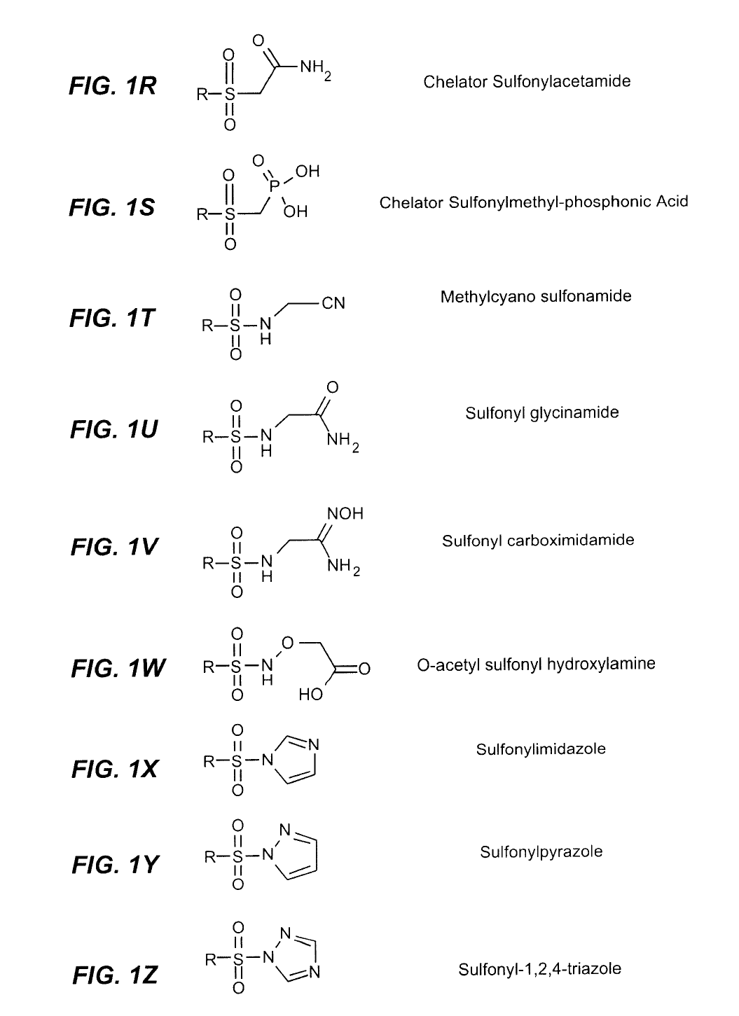

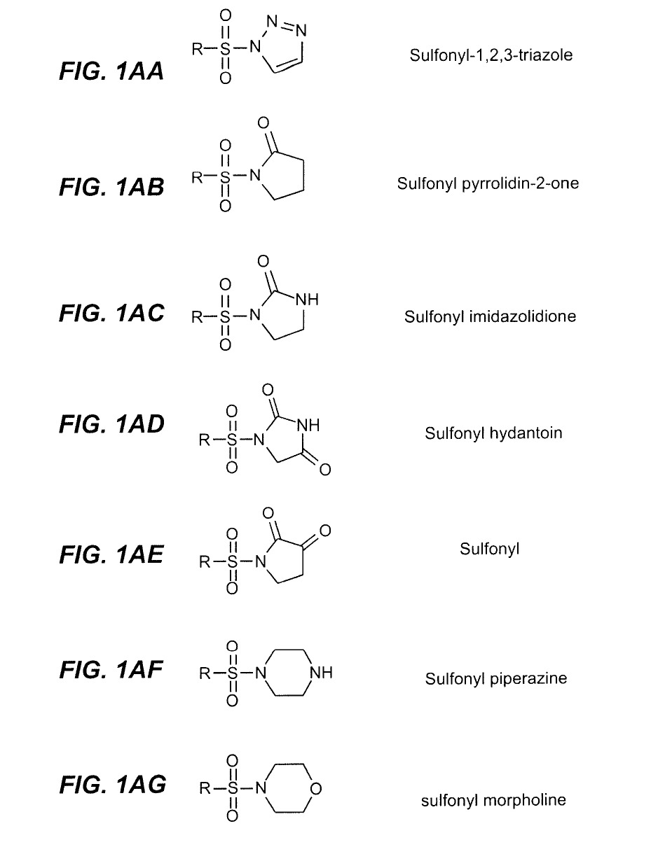

As described herein, the inhibitors of the invention comprise one or more targeting moieties and one or more metal binding moieties. As will be appreciated by those in the art, specific inhibitors of the invention comprise any of the targeting moieties outlined herein joined with an optional linker to any of the metal binding moieties outlined herein, such as those of the figures. Thus, FIG. 1A structures can be joined with FIG. 24(1) structures, etc. In addition, any of the targeting moieties can be joined with classes and/or subclasses of metal binding moieties, to form inhibitors to be tested for specific enzymatic properties such as Ki.

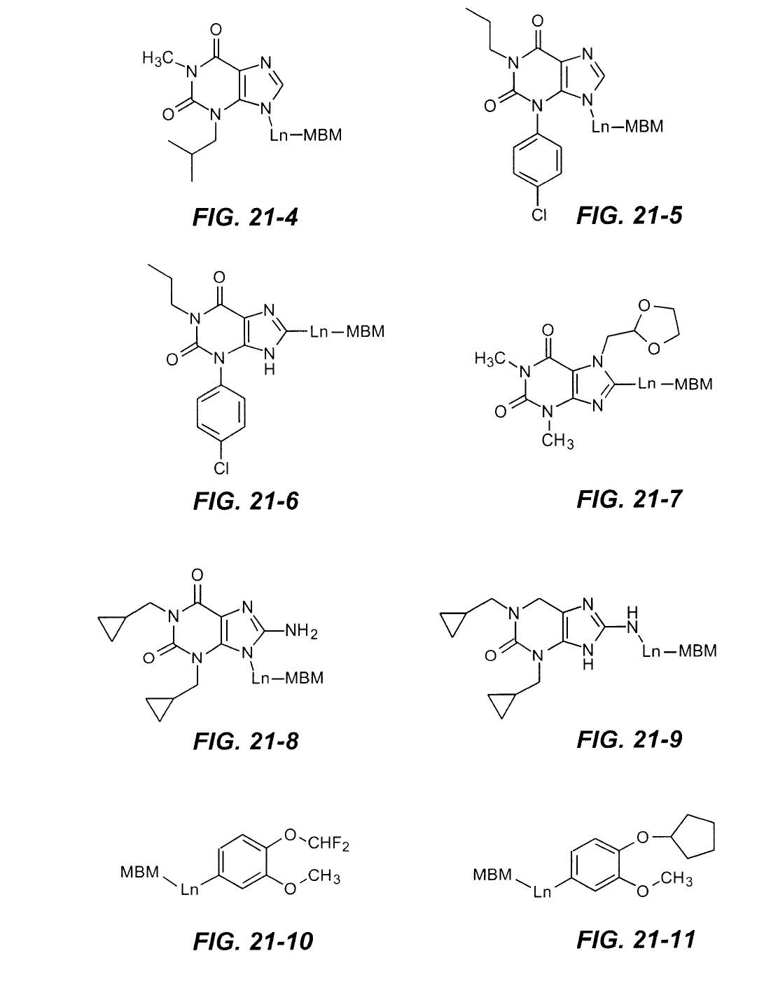

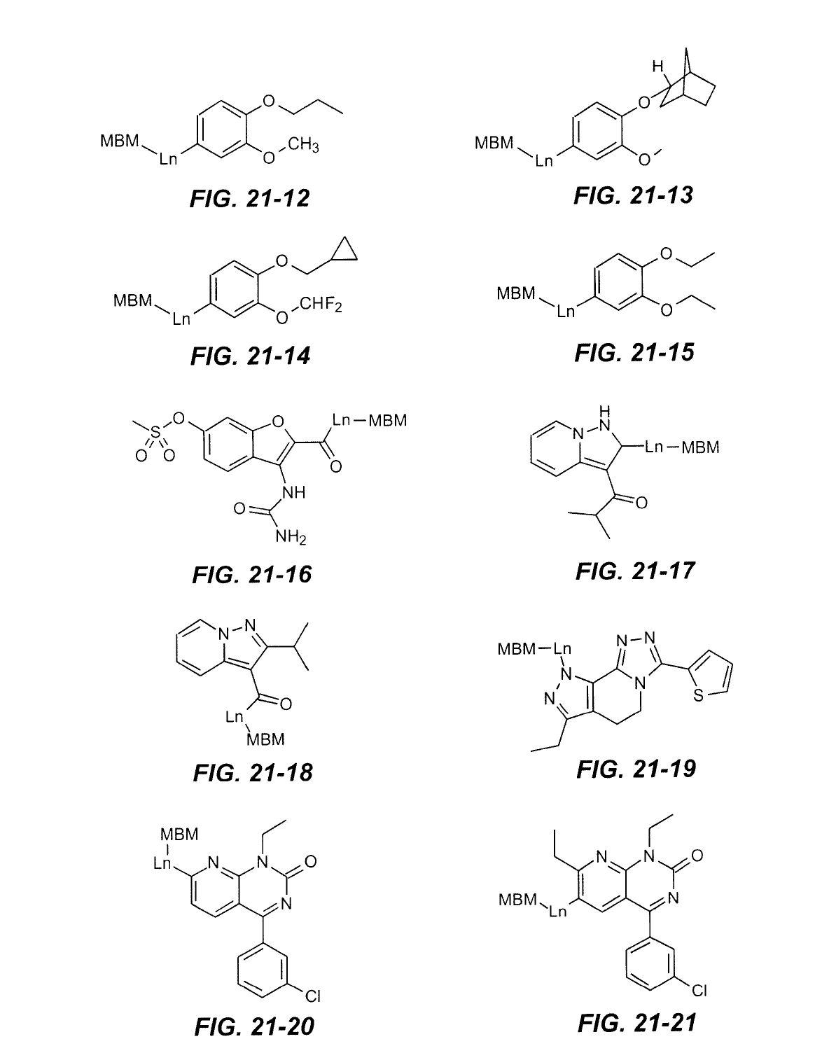

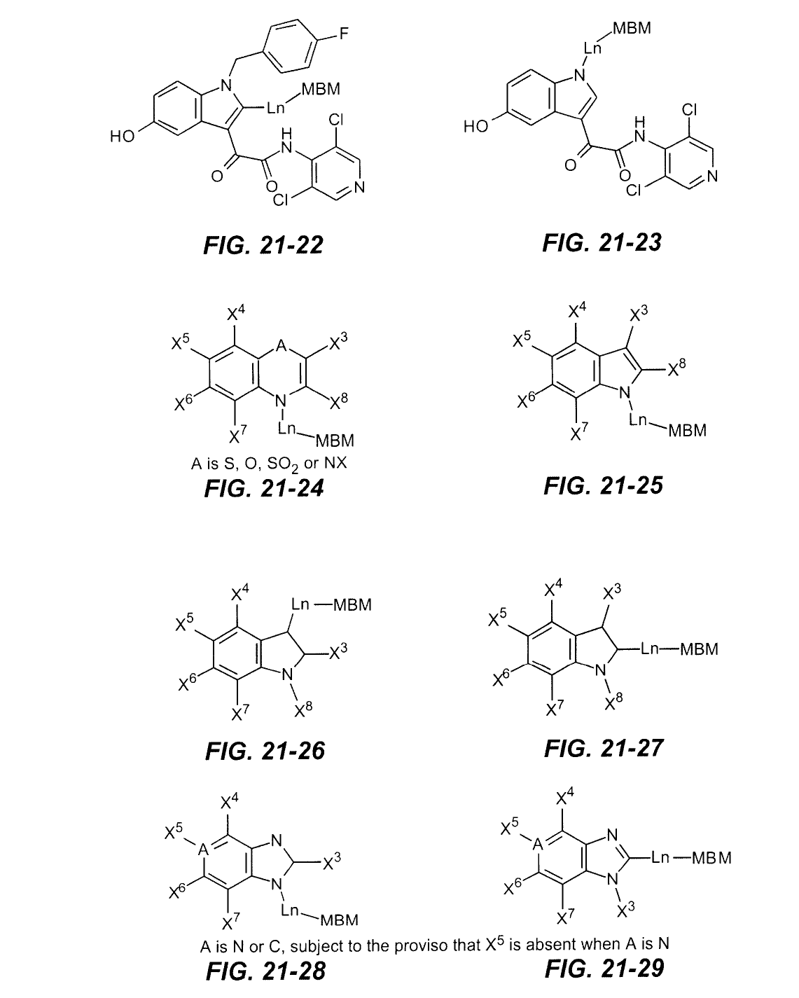

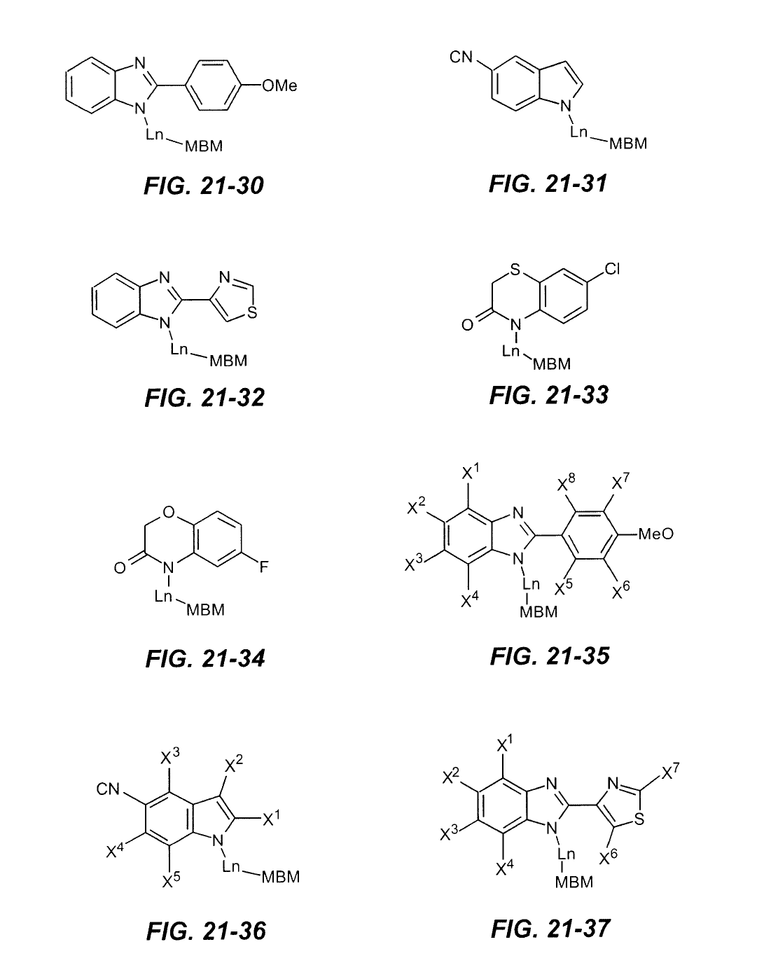

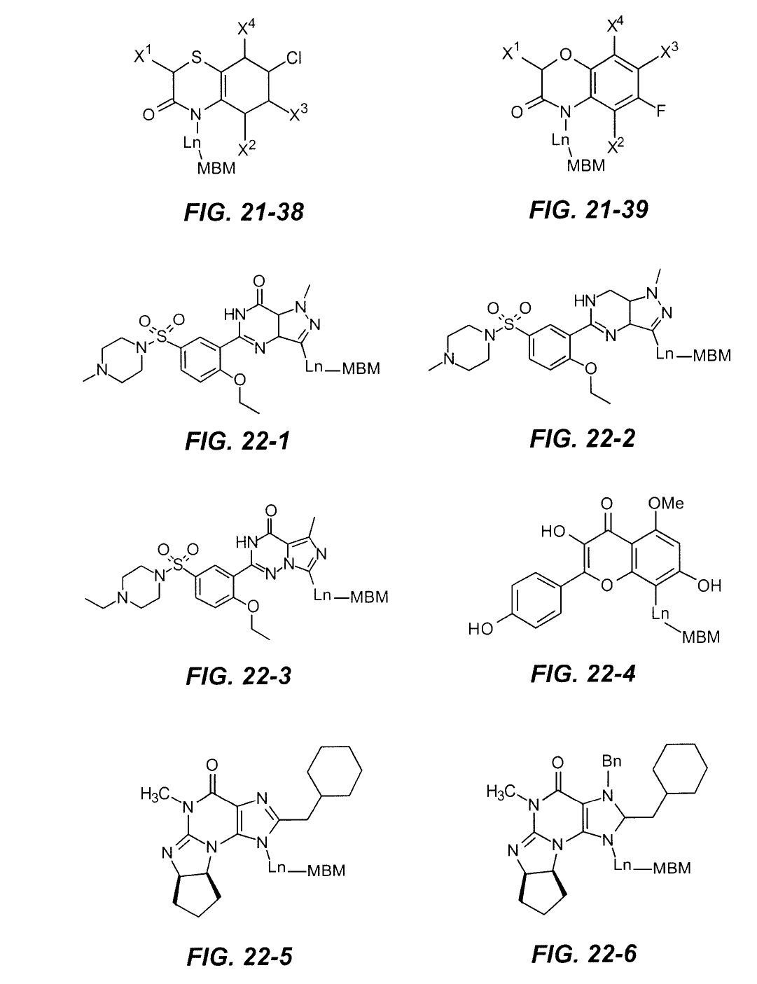

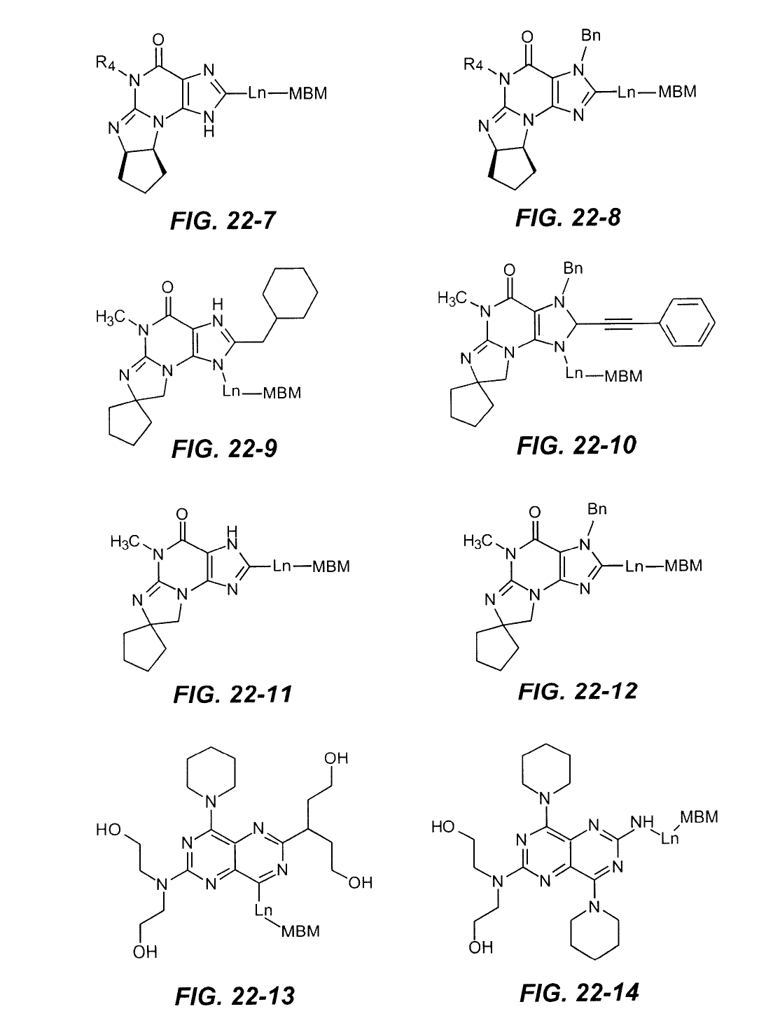

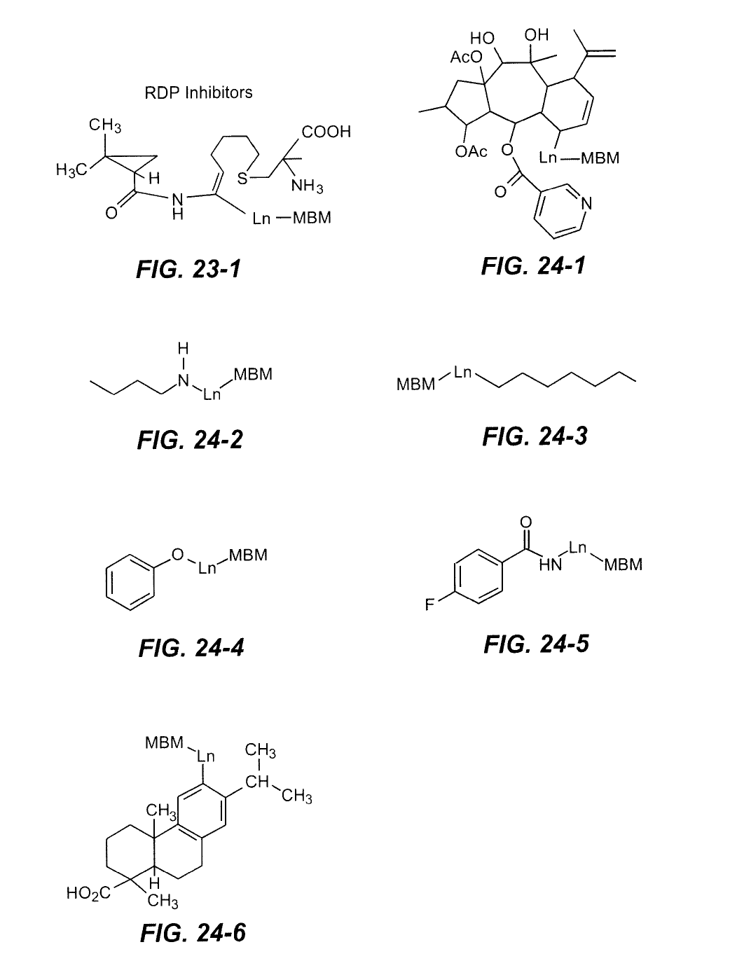

Thus, for example, any independently selected metal binding moiety, or class or subclass of metal binding moiety listed in Figures can be added to any independently selected targeting moiety. For example, 5 membered aromatic rings with heteroatoms can be added to any independently selected PDE4 inhibitor depicted in FIG. 21. Any and all combinations and subcombinations of any size are contemplated.

### Production of Hydrolases

Hydrolase proteins of the present invention may be shorter or longer than protein sequences described by the NCBI databases. Thus, in a preferred embodiment, included within the definition of hydrolase proteins are portions or fragments of the sequences described in NCBI databases, which are all herein expressly incorporated by reference. Portions or fragments of hydrolase proteins are considered hydrolase proteins if a) they share at least one antigenic epitope; or b) have at least the indicated homology; or c) preferably have hydrolase biological activity, e.g., if it is PDE4, including, but not limited to phosphodiesterase activity; and d) if it is PDE4, preferably hydrolyze cAMP selectively.

In general, the hydrolase enzymes used to test inhibitors are recombinant. A “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a hydrolase protein from one organism in a different organism or host cell. Alternatively, the protein may be made at a significantly higher concentration than is normally seen, through the use of a inducible promoter or high expression promoter, such that the protein is made at increased concentration levels. Alternatively, the protein may be in a form not normally found in nature, as in the addition of an epitope tag or amino acid substitutions, insertions and deletions, as discussed below.

Also included within the definition of hydrolase proteins of the present invention are amino acid sequence variants. These variants fall into one or more of three classes: substitutional, insertional or deletional variants. These variants ordinarily are prepared by site specific mutagenesis of nucleotides in the DNA encoding the hydrolase protein, using cassette or PCR mutagenesis or other techniques well known in the art, to produce DNA encoding the variant, and thereafter expressing the recombinant DNA in cell culture as outlined above. However, variant hydrolase protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the hydrolase protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed hydrolase variants screened for the optimal combination of desired activity. Techniques for making substitution mutations at predetermined sites in DNA having a known sequence are well known, for example, M13 primer mutagenesis and PCR mutagenesis. Screening of the mutants is done using assays of hydrolase protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the hydrolase protein are desired, substitutions are generally made in accordance with the following chart:

CHART I

| Original | Exemplary |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the hydrolase proteins as needed. Alternatively, the variant may be designed such that the biological activity of the hydrolase protein is altered. For example, glycosylation sites may be altered or removed, or the transmembrane domain may be removed for assay development.

Covalent modifications of hydrolase polypeptides are included within the scope of this invention. One type of covalent modification includes reacting targeted amino acid residues of an hydrolase polypeptide with an organic derivatizing agent that is capable of reacting with selected side chains or the N- or C-terminal residues of an hydrolase polypeptide. Derivatization with bifunctional agents is useful, for instance, for crosslinking hydrolase to a water-insoluble support matrix or surface for use in the method for purifying anti-hydrolase antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the hydrolase polypeptide included within the scope of this invention comprises altering the native glycosylation pattern of the polypeptide. “Altering the native glycosylation pattern” is intended for purposes herein to mean deleting one or more carbohydrate moieties found in native sequence hydrolase polypeptide, and/or adding one or more glycosylation sites that are not present in the native sequence hydrolase polypeptide.

Addition of glycosylation sites to hydrolase polypeptides may be accomplished by altering the amino acid sequence thereof. The alteration may be made, for example, by the addition of, or substitution by, one or more serine or threonine residues to the native sequence hydrolase polypeptide (for O-linked glycosylation sites). The hydrolase amino acid sequence may optionally be altered through changes at the DNA level, particularly by mutating the DNA encoding the hydrolase polypeptide at preselected bases such that codons are generated that will translate into the desired amino acids.

Another means of increasing the number of carbohydrate moieties on the hydrolase polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the hydrolase polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of hydrolase comprises linking the hydrolase polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Hydrolase polypeptides of the present invention may also be modified in a way to form chimeric molecules comprising an hydrolase polypeptide fused to another, heterologous polypeptide or amino acid sequence. In one embodiment, such a chimeric molecule comprises a fusion of an hydrolase polypeptide with a tag polypeptide which provides an epitope to which an anti-tag antibody can selectively bind. The epitope tag is generally placed at the amino-or carboxyl-terminus of the hydrolase polypeptide. The presence of such epitope-tagged forms of an hydrolase polypeptide can be detected using an antibody against the tag polypeptide. Also, provision of the epitope tag enables the hydrolase polypeptide to be readily purified by affinity purification using an anti-tag antibody or another type of affinity matrix that binds to the epitope tag. In an alternative embodiment, the chimeric molecule may comprise a fusion of an hydrolase polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the 17 gene 10 protein peptide tag [Lutz-Freyermuth et al., Proc. Natl. Acad. Sci. USA, 87:6393-6397 (1990)].

Nucleic acids encoding the hydrolase proteins of the invention can be made as is known in the art. Similarly, using these nucleic acids a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the hydrolase proteins. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the hydrolase protein, as will be appreciated by those in the art; for example, transcriptional and translational regulatory nucleic acid sequences from Bacillus are preferably used to express the hydrolase protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences include constitutive and inducible promoter sequences. The promoters may be either naturally occurring promoters, hybrid or synthetic promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors and appropriate selection and screening protocols are well known in the art and are described in e.g., Mansour et al., Cell, 51:503 (1988) and Murray, Gene Transfer and Expression Protocols, Methods in Molecular Biology, Vol. 7 (Clifton: Humana Press, 1991).

In addition, in a preferred embodiment, the expression vector contains a selection gene to allow the selection of transformed host cells containing the expression vector, and particularly in the case of mammalian cells, ensures the stability of the vector, since cells which do not contain the vector will generally die. Selection genes are well known in the art and will vary with the host cell used. By “selection gene” herein is meant any gene which encodes a gene product that confers resistance to a selection agent. Suitable selection agents include, but are not limited to, neomycin (or its analog G418), blasticidin S, histinidol D, bleomycin, puromycin, hygromycin B, and other drugs.

In a preferred embodiment, the expression vector contains a RNA splicing sequence upstream or downstream of the gene to be expressed in order to increase the level of gene expression. See Barret et al., Nucleic Acids Res. 1991; Groos et al., Mol. Cell. Biol. 1987; and Budiman et al., Mol. Cell. Biol. 1988.

A preferred expression vector system is a retroviral vector system such as is generally described in Mann et al., Cell, 33:153-9 (1993); Pear et al., Proc. Natl. Acad. Sci. U.S.A., 90(18):8392-6 (1993); Kitamura et al., Proc. Natl. Acad. Sci. U.S.A., 92:9146-50 (1995); Kinsella et al., Human Gene Therapy, 7:1405-13; Hofmann et al., Proc. Natl. Acad. Sci. U.S.A., 93:5185-90; Choate et al., Human Gene Therapy, 7:2247 (1998); PCT/US97/01019 and PCT/US97/01048, and references cited therein, all of which are hereby expressly incorporated by reference.

The hydrolase proteins of the present invention are produced by culturing a host cell transformed with nucleic acid, preferably an expression vector, containing nucleic acid encoding a hydrolase protein, under the appropriate conditions to induce or cause expression of the hydrolase protein. The conditions appropriate for hydrolase protein expression will vary with the choice of the expression vector and the host cell; and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jurkat cells, mast cells and other endocrine and exocrine cells, and neuronal cells. See the ATCC cell line catalog, hereby expressly incorporated by reference.

In a preferred embodiment, the hydrolase proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for hydrolase protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, hydrolase proteins are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of hydrolase protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the hydrolase protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatargeting moielyent, electroporation, and others.

In one embodiment, hydrolase proteins are produced in insect cells. Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994).

In a preferred embodiment, hydrolase protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

The hydrolase protein may also be made as a fusion protein, using techniques well known in the art. Thus, for example, for the creation of monoclonal antibodies, if the desired epitope is small, the hydrolase protein may be fused to a carrier protein to form an immunogen. Alternatively, the hydrolase protein may be made as a fusion protein to increase expression, or for other reasons. For example, when the hydrolase protein is an hydrolase peptide, the nucleic acid encoding the peptide may be linked to other nucleic acid for expression purposes.

In one embodiment, the hydrolase nucleic acids, proteins and antibodies of the invention are labeled. By “labeled” herein is meant that nucleic acids, proteins and antibodies of the invention have at least one element, isotope or chemical compound attached to enable the detection of nucleic acids, proteins and antibodies of the invention. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

In a preferred embodiment, the hydrolase protein is purified or isolated after expression.

hydrolase proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the hydrolase protein may be purified using a standard anti-hydrolase antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the hydrolase protein. In some instances no purification will be necessary.

Once expressed and purified if necessary, the hydrolase proteins and nucleic acids are useful in a number of applications.

Screening for Hydrolase Inhibitors

Screens may be designed to find targeting moieties that can bind to hydrolase proteins, and then these targeting moieties may be linked to the metal binding moieties to form hydrolase candidate inhibitors and then used in assays that evaluate the ability of the candidate inhibitors to modulate hydrolase bioactivity. Alternatively, targeting moieties can be linked with the metal binding moiety to first screen for binding activity to hydrolases and then screen inhibiting activity, or in opposite order. Thus, as will be appreciated by those in the art, there are a number of different assays which may be run; binding assays and activity assays.

Target Moiety Screening

In a preferred embodiment, the methods comprise combining hydrolase proteins and a candidate targeting moiety, and determining the binding of the targeting moiety to the hydrolase proteins. In general, as described herein, the assays are done by contacting a hydrolase protein with one or more targeting moieties to be tested.

Targeting moieties encompass numerous chemical classes. In one embodiment, the target moeity is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Targeting moieties comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Targeting moieties are also found among biomolecules including peptides, saccharides, fatty acids, steroids, purines, pyrimidines, derivatives, structural analogs or combinations thereof.

Targeting moieties are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the targeting moieties are organic chemical moieties, a wide variety of which are available in the literature.

In a preferred embodiment, the targeting moieties are obtained from combinatorial chemical libraries, a wide variety of which are available in the literature. By “combinatorial chemical library” herein is meant a collection of diverse chemical compounds generated in a defined or random manner, generally, but not always, by chemical synthesis. Millions of chemical compounds can be synthesized through combinatorial mixing.

In a preferred embodiment, the targeting moiety is a carbohydrate. By “carbohydrate” herein is meant a compound with the general formula Cx(H₂O)y. Monosaccharides, disaccharides, and oligo- or polysaccharides are all included within the definition and comprise polymers of various sugar molecules linked via glycosidic linkages. Particularly preferred carbohydrates are those that comprise all or part of the carbohydrate component of glycosylated proteins, including monomers and oligomers of galactose, mannose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates. In particular, polysaccharides (including, but not limited to, arabinogalactan, gum arabic, mannan, etc.) have been used to deliver MRI agents into cells; see U.S. Pat. No. 5,554,386, hereby incorporated by reference in its entirety.

In a preferred embodiment, the targeting moiety is a lipid. “Lipid” as used herein includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

In a preferred embodiment, the targeting moieties are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations. Peptide inhibitors of hydrolase enzymes find particular use.

In a preferred embodiment, the targeting moieties are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

In some embodiments, the candidate agents are peptides. In this embodiment, it can be useful to use peptide constructs that include a presentation structure. By “presentation structure” or grammatical equivalents herein is meant a sequence, which, when fused to candidate bioactive agents, causes the candidate agents to assume a conformationally restricted form. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present randomized peptide structures. Preferred presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc. See U.S. Pat. No. 6,153,380, incorporated by reference.

Of particular use in screening assays are phage display libraries; see e.g., U.S. Pat. Nos. 5,223,409; 5,403,484; 5,571,698; and 5,837,500, all of which are expressly incorporated by reference in their entirety for phage display methods and constructs.

In a preferred embodiment, the targeting moieties are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized targeting moieties.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In a preferred embodiment, as is more fully outlined below, the candidate agents are either randomized proteins (including biased proteins or proteins with fusion partners) or expression products of cDNA libraries or libraries derived from cDNA libraries, such as fragmented (including randomly fragmented cDNA libraries). These are added to the cells as nucleic acids encoding these proteins. As will be appreciated by those in the art, these cDNA libraries may be full length or fragments, and can be in-frame, out-of-frame or read from the anti-sense strand.

In a preferred embodiment, the targeting moiety is an antibody. The term “antibody” includes antibody fragments, as are known in the art, including Fab Fab₂, single chain antibodies (Fv for example), chimeric antibodies, etc., either produced by the modification of whole antibodies or those synthesized de novo using recombinant DNA technologies.

In a preferred embodiment, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework. residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fe), typically that of a human immunoglobulin [Jones et al., Nature 321:522-525 (1986); Riechmann et al., Nature 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321:522-525 (1986); Riechmann et al., Nature 332:323-327 (1988); Verhoeyen et al., Science 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol. 227:381 (1991); Marks et al., J. Mol. Biol. 222:581 (1991)]. The techniques of Cole et al. and Boerner et at. are also available for the preparation of human monoclonal antibodies (Cole et al., Monoclonal Antibodies and Cancer Therapy, Alan R. Liss, p. 77 (1985) and Boemer et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779.783 (1992); Lonberg et al., Nature 368:856-859 (1:994); Morrison, Nature 368:812-13 (1994); Fishwild et al., Nature Biotechnology 14:845-51 (1996); Neuberger, Nature Biotechnology, 14:826 (1996); Lonberg and Huszar, Intern. Rev. Immunol. 13:65-93 (1995).

Bispecific antibodies are monoclonal, preferably human or humanized, antibodies that have binding specificities for at least two different antigens. In the present case, one of the binding specificities is for a first target molecule and the other one is for a second target molecule.

Methods for making bispecific antibodies are known in the art. Traditionally, the recombinant production of bispecific antibodies is based on the co-expression of two immunoglobulin heavy-chairi/light-chain pairs, where the two heavy chains have different specificities [Milstein and Cuello. Nature 305:537-539 (1983)]. Because of the random assortargeting moietyent of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology 121:210 (1986).

Heteroconjugate antibodies are also within the scope of the present invention.

Heteroconjugate antibodies are composed of two covalently joined antibodies. Such antibodies have, for example, been proposed to target immune system cells to unwanted cells [U.S. Pat. No. 4,676,980], and for treatargeting moietyent of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In a preferred embodiment, the candidate bioactive agents are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen. Nature, 365:566 (1993); Carlsson, et al., Nature, 380:207 (1996), all of which are incorporated by reference)). Other analog nucleic acids include those with positive backbones (Denpcy. et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3. ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook, and peptide nucleic acids. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occuring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. etc.

In one embodiment, the nucleic acids are aptamers, see U.S. Pat. Nos. 5,270,163, 5,475,096, 5,567,588, 5,595,877, 5,637,459, 5,683,867, 5,705,337, and related patents, hereby incorporated by reference.

It should be noted in the context of the invention that nucleosides (ribose plus base) and nucleotides (ribose, base and at least one phosphate) are used interchangeably herein unless otherwise noted.

As described above generally for proteins, nucleic acid targeting moieties may be naturally occurring nucleic acids, random and/or synthetic nucleic acids, or “biased” random nucleic acids. For example, digests of prokaryotic or eukaryotic genomes may be used as is outlined above for proteins.

In a preferred embodiment, a library of different targeting moieties are used. Preferably, the library should provide a sufficiently structurally diverse population of randomized agents to effect a probabilistically sufficient range of diversity to allow binding to a particular target. Accordingly, an interaction library should be large enough so that at least one of its members will have a structure that gives it affinity for the target. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷ to 10⁸ is sufficient to find structures with affinity for the target. A library of all combinations of a peptide 7 to 20 amino acids in length, such as generally proposed herein, has the potential to code for 20⁷ (10⁹) to 20²⁰. Thus, with libraries of 10⁷ to 10⁸ different molecules the present methods allow a “working” subset of a theoretically complete interaction library for 7 amino acids, and a subset of shapes for the 20²⁰ library. Thus, in a preferred embodiment, at least 10⁶, preferably at least 10⁷, more preferably at least 10⁶ and most preferably at least 10⁹ different sequences are simultaneously analyzed in the subject methods. Preferred methods maximize library size and diversity.

Once expressed and purified, if necessary, the hydrolase proteins are used in screening assays for the identification of hydrolase candidate inhibitors comprising Metal binding moieties and targeting moieties that bind to the hydrolase proteins and inhibit hydrolase activity.

In a preferred embodiment, the targeting moieties are screened first by using candidate agents as outlined herein for their desired properties and then linked to the metal binding moiety to form hydrolase candidate inhibitors for further screening using the method provided in the present invention.

In another preferred embodiment, the targeting moiety are not pre-screened. The targeting moieties are linked to the metal binding moiety, then are used for screening using the method provided in the present invention.

The targeting moieties are contacted with the hydrolase protein under reaction conditions that favor agent-target interactions. Generally, this will be physiological conditions. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away, in the case of solid phase assays. Assay formats are discussed below.

A variety of other reagents may be included in the assays. These include reagents like salts, neutral proteins, e.g. albumin, detergents, etc which may be used to facilitate optimal hydrolase protein-targeting moiety binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

In one embodiment, solution phase binding assays are done. Generally in this embodiment, fluorescence resonance energy transfer (FRET) assays are done, by labeling both the targeting moieties and hydrolase proteins with different fluorophores with overlapping spectra. As energy transfer is distance dependent, in the absence of binding the excitation at one wavelength does not produce an emission spectra. Only if the two labels are close, e.g. when binding has occurred, will excitation at one wavelength result in the desired emission spectra of the second label.

In some embodiments, solid phase (heterogeneous) assays are done. In this case, binding assays are done wherein either the hydrolase protein or the targeting moiety is non-diffusably bound to an insoluble solid support, and detection is done by adding the other component which is labeled, as described below.

The insoluble supports may be made of any composition to which the compositions can be bound, is readily separated from soluble material, and is otherwise compatible with the overall method of screening. The surface of such supports may be solid or porous and of any convenient shape. Examples of suitable supports include microtiter plates, arrays, membranes and beads, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica based materials including silicon and modified silicon, carbon, metals, inorganic glasses, plastics, ceramics, and a variety of other polymers. In a some embodiments, the solid supports allow optical detection and do not themselves appreciably fluoresce. In addition, as is known the art, the solid support may be coated with any number of materials, including polymers, such as dextrans, acrylamides, gelatins, agarose, etc. Exemplary solid supports include silicon, glass, polystyrene and other plastics and acrylics. Microliter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable.

In a preferred embodiment, the hydrolase protein is bound to the support, and a library of targeting moieties are added to the assay. Alternatively, the targeting moiety is bound to the support and the hydrolase protein is added. Attachment to the solid support is accomplished using-well known methods, and will depend on the composition of the two materials to be attached. In general, for covalent attachment, attachment linkers are utilized through the use of functional groups on each component that can then be used for attachment. Preferred functional groups for attachment are amino groups, carboxy groups, oxo groups, hydroxyl groups and thiol groups. These functional groups can then be attached, either directly or indirectly through the use of a linker. Linkers are well known in the art; for example, homo-or hetero-bifunctional linkers as are well known (see 1994 Pierce Chemical Company catalog, technical section on cross-linkers, pages 155-200, incorporated herein by reference). In some embodiments, absorption or ionic interactions are utilized. In some cases, small molecule candidate agents are synthesized directly on microspheres, for example, which can then be used in the assays of the invention.

Following binding of the protein or targeting moiety, excess unbound material is removed by washing. The surface may then be blocked through incubation with bovine serum albumin (BSA), casein or other innocuous protein or other moiety.

In the binding assays, either the hydrolase protein, the targeting moiety (or, in some cases, the metal binding moiety, or substrate of hydrolase enzymes, described below) is labeled. By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

Specific labels include optical dyes, including, but not limited to, chromophores, phosphors and fluorophores, with the latter being specific in many instances. Fluorophores can be either “small molecule” fluores, or proteinaceous fluores as described above. The labeled metal donor (e.g. the metal binding component) can be a chemical probe (such as Zinquin or Zinbo5) which undergoes a spectroscopic change when it releases the metal ion as described herein.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, hereby expressly incorporated by reference.

In one embodiment, the hydrolase protein is attached to the support, adding labeled targeting moiety, washing off excess reagent, and determining whether the label is present on the solid support. Various blocking and washing steps may be utilized as is known in the art.

In one embodiment, the targeting moieties are immobilized to the support, and a labeled hydrolase protein is added to determine binding.

Activity assays are done as are known in the art.

### Screening for PDE4 Inhibitors

For example, when inhibitors of PDE4 is screened, the screening is done by directly assaying the ability of PDE4 candidate inhibitors to inhibit PDFE4 enzymes activity. There are a variety of assays that could be used to assay the activity of PDE4 enzymes. See Lugnier, Phosphodiesterase Methods and Protocols (Humana Press, 2005), hereby incorporated by reference.

In some embodiments, bioactivity assays are done to test whether the PDE4 candidate inhibitor inhibits PDE4 enzymes bioactivity. As for binding assays, activity assays can be either solution based, or rely on the use of components that are immobilized on solid supports. In this case, the bioactivity assay depends on the bioactivity of the PDE4 enzymes, and will be run accordingly. Thus, for example, PDE4 enzymes activity assays are well known, using a wide variety of generally commercially available substrates, such as cAMP or its derivatives. Generally a plurality of assay mixtures are run in parallel with different PDE4 inhibitor candidates concentrations to obtain a differential response to the various concentrations. Typically, one of these concentrations serves as a negative control, i.e., at zero concentration or below the level of detection.

In one embodiment, the methods comprise contacting the candidate inhibitor with PDE4 enzymes. The candidate inhibitor and PDE4 enzymes can be added simultaneously or sequentially.

In one embodiment. the PDE4 enzymes are naturally occurred, expressed by a cell line that expressly PDE4 enzymes. In another preferred embodiment, the PDE4 enzymes could also be expressed from a recombinant vector carrying the whole PDE4 genes or part of it, being transformed or transferred into host cells, integrated or not integrated in the chromosomes of the host cells. When PDE4 enzymes are produced as recombinant proteins from host cells, they could reside within the cell, or be secreted to the outside of the cells.

In one preferred embodiment, the PDE4 enzymes are not purified.

In another preferred embodiment, the PDE4 enzymes are purified, or partial purified, either from sources having nature occurred PDE4 enzymes, or recombinant PDE4 enzymes.

In a preferred embodiment, the assay for PDE4 activity is done by adding PDE4 candidate inhibitors to a cell culture expressing nature occurred or recombinant PDE4 enzymes.

In another preferred embodiment, the assay for PDE4 activity is done by mixing PDE4 candidate inhibitors with purified PDE4 enzymes in vitro.

A variety of other reagents may be included in the screening assays. These include reagents like salts, neutral proteins, e.g. albumin, detergents, etc which may be used to facilitate optimal PDE4 enzyme activity and/or reduce non-specific or background actions. Also reagents that otherwise improve the efficiency of the assay, such as protease inhibitors, nuclease inhibitors, anti-microbial agents, etc., may be used. The mixture of components may be added in any order that provides for the requisite assay.

Positive controls and negative controls may be used in the assays. Preferably all control and test samples are performed in at least triplicate to obtain statistically significant results. Incubation of all samples is for a time sufficient for PDE4 enzymes to act. Following incubation, all reactions are terminated by adding reaction termination agent, such as EDTA or other detergent to deactivate PDE4 enzymes. Other method such as heating could also be used to inactive PDE4 enzymes.

In a preferred embodiment, a PDE4 enzymes substrate is in contact with the PDE4 enzymes and/or the PDE4 candidate inhibitors.

In a preferred embodiment, for the test assay, PDE4 enzymes and PDE4 candidate inhibitors are in contact first, preferably after a period of pre-incubation, then are in contact with substrate; and for the control assay, PDE4 enzymes are in contact with substrate directly. In another preferred embodiment, PDE4 candidate inhibitors are in contact with substrate first, then are in contact with PDE4 enzymes; and for the control assay, substrate is in contact with PDE4 enzymes directly.

In a preferred embodiment, a “positive control” and/or a “negative control” could be used to control the reliability and quality of the assay. A positive control is an assay essentially same to an assay to test the effect of PDE4 candidate inhibitor except that the PDE4 candidate inhibitor is replaced by a known PDE4 inhibitor. One known PDE4 specific inhibitor is rolipram. A negative control is an assay essentially same to an assay to test the effect of PDE4 candidate inhibitor except that the PDE4 candidate inhibitor is replaced by a known PDE4 non-inhibitor. In another preferred embodiment, a plurality of positive controls and/or negative controls is used.

The activity of PDE4 enzymes could be measured by their ability to catalyze a substrate. By “substrate” herein meant a molecule that PDE4 enzymes are capable of acting upon. When substrate are in contact PDE4 enzymes, PED4 would catalyze a chemical reactions that involves the substrate that generally lead to some change to the substrate, or preferably, converts the substrate into a different molecule. Thus any molecule that PDE4 enzymes could act upon is a substrate, and preferably, selectively. One known PDE4 specific substrate is cAMP. Though many derivatives of cAMP through chemical or biological modification could also be specific substrate and be suited to the present invention. A substrate could be cAMP, a cAMP derivative. or a cAMP analogue. In one preferred embodiment, the substrate is cAMP.

In a preferred embodiment, substrate, such as cAMP or one of its derivatives, is directly or indirectly labeled to provide detectable signal as described above. For example, a radioisotope (such as ³H, ¹⁴C, ³²P, ³³P, ³⁵S, or ¹²⁵I), a fluorescent or chemiluminescent compound (such as fluorescein isothiocyanate, rhodamine, or luciferin), an enzyme (such as alkaline phosphatase, beta-galactosidase or horseradish peroxidase), antibodies, particles such as magnetic particles, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal. A more complete list of flurophores are provided in the section of Targeting moiety.

In one preferred embodiment, the substrate is cAMP, which could be naturally occurred or synthesized.

Hydrolysis of cAMP by PDE4 enzymes could be measured by the decrease of cAMP or the increase of the hydrolysis product, AMP. This could be done by comparing an assay wherein PDE4 enzymes are in contact with PDE4 candidate inhibitors (“test assay”) and an assay wherein the PDE4 enzymes are not in contact with PDE4 inhibitors (“control assay”). The later could also be called a control. The test assay and control assay are carried out under the same condition unless otherwise particularly described herein. Thus the cAMP in the control assay will decrease comparing to the control assay, while there is increase of AMP or other molecules resulted due the activity of PDE4 enzymes. In contrast, in the test assay, due to the presence of the PDE4 candidate inhibitor, which is capable of inhibiting the activity of PDE4 enzymes, the cAMP in the control assay will not decrease and there is not or AMP or other molecules resulted from the hydrolysis by PDE4 enzymes after a period of time to allow the enzyme to act.

In a preferred embodiment, the activity of PDE4 enzymes is measured by the decrease of substrate. This could be done by comparing the amount of substrate in the assay sample before and after a period of time to allow the enzymes to act.

In a preferred embodiment, the activity of PDE4 enzymes is measured by the decrease of cAMP. This could be done by comparing the amount of cAMP in the assay sample before after a period of time to allow the enzyme to act.

In a preferred embodiment, the activity of PDE4 enzymes is measured by the increase of PDE4 enzymes hydrolysis product. By “hydrolysis product” herein is meant the molecules resulted from the hydrolysis of the substrate by PDE4 enzymes, or molecules resulted from one or more down stream reaction following the hydrolysis of substrate by PDE4 enzymes. For example, when the substrate is cAMP, the hydrolysis product is AMP, or adenosine, which is converted from the AMP by further down stream reaction.

In one preferred embodiment, PDE4 enzymes activity is determined by the amount of adenosine after the reaction. In this embodiment, PDE4 enzymes are incubated with labeled cAMP, with or without PDE4 candidate inhibitor, in a buffer and at a temperature proper for PDE4 enzymes activity. After a desired period of time, the reaction is stopped by heating at high temperature, such as 100 degree for a period of time, preferably three minutes, to inactive the PDE4 enzymes. After cooling the sample to lower temperature, a second agent that could convert AMP to a different form is added. In a preferred embodiment, the agent is alkaline phosphate. The agent could also be an enzyme. After another incubation in a proper buffer, under proper temperature, and for a desired period of time, the reaction is stopped, such as by heating at high temperature for a period of time. Then the adenosines, if there are any, could be separated from cAMP and AMP using standard method known in the art. In a preferred embodiment, adenosine is separated from cAMP and AMP using an affinity column. After such separation, the amount of adenosine is then measured to determine the activity of PDE4 enzymes and the ability of PDE4 candidate inhibitor to inhibit PDE4 enzyme activity.

In another preferred embodiment, the screening is done by a competition assay. In such assay, a known PDE4 inhibitor, such as rolipram is used. Rolipram is used in an assay to inhibit PDE4 enzyme activity. Then in parallel assays, PDE4 candidate inhibitors are screened by replacing rolipram in the otherwise same assay.

In one preferred embodiment, a plurality of PDE4 candidates could be used in combination according to a matrix to form mixtures, and the mixtures are used to test the ability to inhibit PDE4 enzyme activity. For example, a hundred of PDE4 candidate inhibitors could be assigned to a 10×10 matrix, and each column and row is mixed and tested for ability to inhibit PDE4 enzyme activities. There are thus total 20 samples to test. Then the test results are plotted against the matrix, and any double-positive in the matrix will be a positive result for PDE4 candidate inhibitors. This matrix thus could speed up the screen process. It could also be expended into more than two dimensions, such as three, four, or five dimensions.

In one embodiment, the candidate inhibitors are also tested against other enzymes, particularly other PDE enzymes, for specificity.

In one embodiment, any of the assays outlined herein can utilize robotic systems for high throughput screening. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

As will be appreciated by those in the art, there are a wide variety of components which may be used, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfiuidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, minitubes. deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4° C. to 100° C.

In some preferred embodiments, the instrumentation will include a detector, which may be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluroescence resonance energy transfer (FRET), SPR systems, luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

These instruments can fit in a sterile laminar flow or fume hood, or are enclosed, self-contained systems as needed. Flow cytometry or capillary electrophoresis formats may be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic workstation includes one or more heating or cooling components. Depending on the reactions and reagents, either cooling or heating may be required, which may be done using any number of known heating and cooling systems, including Peltier systems.

In a preferred embodiment, the robotic apparatus includes a central processing unit that communicates with a memory and a set of input/output devices (e.g., keyboard, mouse, monitor, printer, etc.) through a bus. The general interaction between a central processing unit, a memory, input/output devices, and a bus is known in the art. Thus, a variety of different procedures, depending on the experiments to be run, are stored in the CPU memory.

### Pharmaceutical Compositions and Methods of Treatment

As previously discussed, the inhibitors of the invention inhibit the activity of metallo-proteases. As a consequence of these activities, the active compounds of the invention may be used in a variety of in vitro, in vivo and ex vivo contexts to inhibit activity, particularly in cases where metallo-protease activity is implicated in disease states.

When used to treat or prevent such diseases, the active compounds may be administered singly, as mixtures of one or more active compounds or in mixture or combination with other agents useful for treating such diseases and/or the symptoms associated with such diseases. The active compounds may also be administered in mixture or in combination with agents useful to treat other disorders. The active compounds may be administered per se in the form of prodrugs or as pharmaceutical compositions, comprising an active compound or prodrug.

Pharmaceutical compositions comprising the active compounds of the invention (or prodrugs thereof) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the active compounds into preparations which can be used pharmaceutically.

The active compound or prodrug may be formulated in the pharmaceutical compositions per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions of the invention may take a form suitable for virtually any mode of administration, including, for example, topical, ocular, oral, baccal, systemic, nasal, injection, transdermal, rectal, vaginal, etc., or a form suitable for administration by inhalation or insufflation.

For topical administration, the active compound(s) or prodrug(s) may be formulated as solutions, gels. ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the active compound(s) may dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol or fractionated vegetable oils); and preservatives (e.g., methyl or propyl p hydroxybenzoates or sorbic acid). The preparations may also contain buffer salts, preservatives, flavoring, coloring and sweetening agents as appropriate.

Preparations for oral administration may be suitably formulated to give controlled release of the active compound or prodrug, as is well known.

For buccal administration, the compositions may take the form of tablets or lozenges formulated in conventional manner.

For rectal and vaginal routes of administration, the active compound(s) may be formulated as solutions (for retention enemas) suppositories or ointments containing conventional suppository bases such as cocoa butter or other glycerides.

For nasal administration or administration by inhalation or insufflation, the active compound(s) or prodrug(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. in the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

A specific example of an aqueous suspension formulation suitable for nasal administration using commercially-available nasal spray devices includes the following ingredients: active compound or prodrug (0.5-20 mg/ml); benzalkonium chloride (0.1-0.2 mg/mL); polysorbate 80 (TWEEN® 80; 0.5-5 mg/ml); carboxymethylcellulose sodium or microcrystalline cellulose (1-15 mg/ml); phenylethanol (1-4 mg/ml); and dextrose (20-50 mg/ml). The pH of the final suspension can be adjusted to range from about pH5 to pH7, with a pH of about pH 5.5 being typical.

For ocular administration, the active compound(s) or prodrug(s) may be formulated as a solution, emulsion, suspension, etc. suitable for administration to the eye. A variety of vehicles suitable for administering compounds to the eye are known in the art. Specific non-limiting examples are described in U.S. Pat. No. 6,261,547; U.S. Pat. No. 6,197,934; U.S. Pat. No. 6,056,950; U.S. Pat. No. 5,800,807; U.S. Pat. No. 5,776,445; U.S. Pat. No. 5,698,219; U.S. Pat. No. 5,521,222; U.S. Pat. No. 5,403,841; U.S. Pat. No. 5,077,033; U.S. Pat. No. 4,882,150; and U.S. Pat. No. 4,738,851.

For prolonged delivery, the active compound(s) or prodrug(s) can be formulated as a depot preparation for administration by implantation or intramuscular injection. The active ingredient may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) for percutaneous absorption may be used. To this end, permeation enhancers may be used to facilitate transdermal penetration of the active compound(s). Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems may be employed. Liposomes and emulsions are well-known examples of delivery vehicles that may be used to deliver active compound(s) or prodrug(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration.

The active compound(s) or prodrug(s) of the invention, or compositions thereof, will generally be used in an amount effective to achieve the intended result, for example in an amount effective to treat or prevent the particular disease being treated. The compound(s) may be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a compound to a patient suffering from an allergy provides therapeutic benefit not only when the underlying allergic response is eradicated or ameliorated, but also when the patient reports a decrease in the severity or duration of the symptoms associated with the allergy following exposure to the allergen. As another example, therapeutic benefit in the context of asthma includes an improvement in respiration following the onset of an asthmatic attack, or a reduction in the frequency or severity of asthmatic episodes. Therapeutic benefit also includes halting or slowing the progression of the disease, regardless of whether improvement is realized.

For prophylactic administration, the compound may be administered to a patient at risk of developing one of the previously described diseases. For example, if it is unknown whether a patient is allergic to a particular drug, the compound may be administered prior to administration of the drug to avoid or ameliorate an allergic response to the drug. Alternatively, prophylactic administration may be applied to avoid the onset of symptoms in a patient diagnosed with the underlying disorder. For example, a compound may be administered to an allergy sufferer prior to expected exposure to the allergen. Compounds may also be administered prophylactically to healthy individuals who are repeatedly exposed to agents known to one of the above-described maladies to prevent the onset of the disorder. For example, a compound may be administered to a healthy individual who is repeatedly exposed to an allergen known to induce allergies, such as latex, in an effort to prevent the individual from developing an allergy. Alternatively, a compound may be administered to a patient suffering from asthma prior to partaking in activities which trigger asthma attacks to lessen the severity of, or avoid altogether, an asthmatic episode.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Effective dosages may be estimated initially from in vitro assays. For example, an initial dosage for use in animals may be formulated to achieve a circulating blood or serum concentration of active compound that is at or above an IC50 of the particular compound as measured in as in vitro assay, such as the in vitro CHMC or BMMC and other in vitro assays described in the Examples section. Calculating dosages to achieve such circulating blood or serum concentrations taking into account the bioavailability of the particular compound is well within the capabilities of skilled artisans. For guidance, see Fingl & Woodbury, “General Principles,” In: Goodman and Gilman's The Pharmaceutical Basis of Therapeutics, Chapter 1, pp. 1-46, latest edition, Pagamonon Press, and the references cited therein.

Initial dosages can also be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but may be higher or lower, depending upon, among other factors, the activity of the compound, its bioavailability, the mode of administration and various factors discussed above. Dosage amount and interval may be adjusted individually to provide plasma levels of the compound(s) which are sufficient to maintain therapeutic or prophylactic effect. In cases of local administration or selective uptake, such as local topical administration, the effective local concentration of active compound(s) may not be related to plasma concentration. Skilled artisans will be able to optimize effective local dosages without undue experimentation.

The compound(s) may be administered once per day, a few or several times per day, or even multiple times per. day, depending upon, among other things, the indication being treated and the judgment of the prescribing physician.

Preferably, the compound(s) will provide therapeutic or prophylactic benefit without causing substantial toxicity. Toxicity of the compound(s) may be determined using standard pharmaceutical procedures. The dose ratio between toxic and therapeutic (or prophylactic) effect is the therapeutic index. Compounds(s) that exhibit high therapeutic indices are preferred.

1-21. (canceled)

22. An inhibitor of a PDE4 enzyme having a formula selected from the group consisting of: 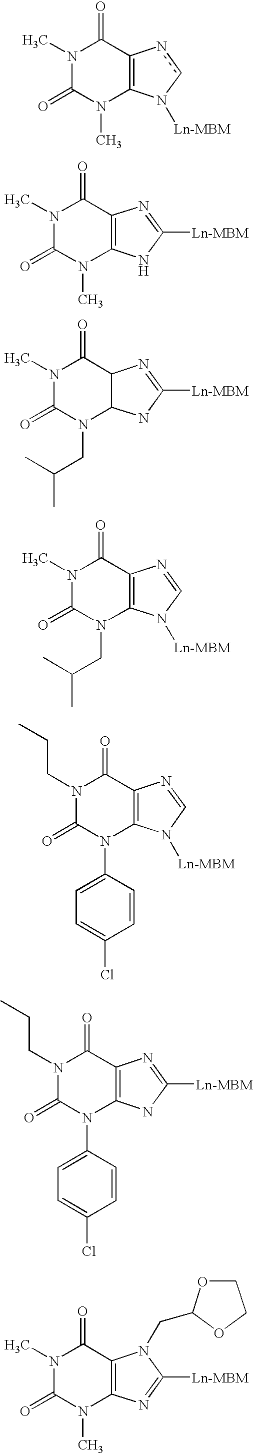 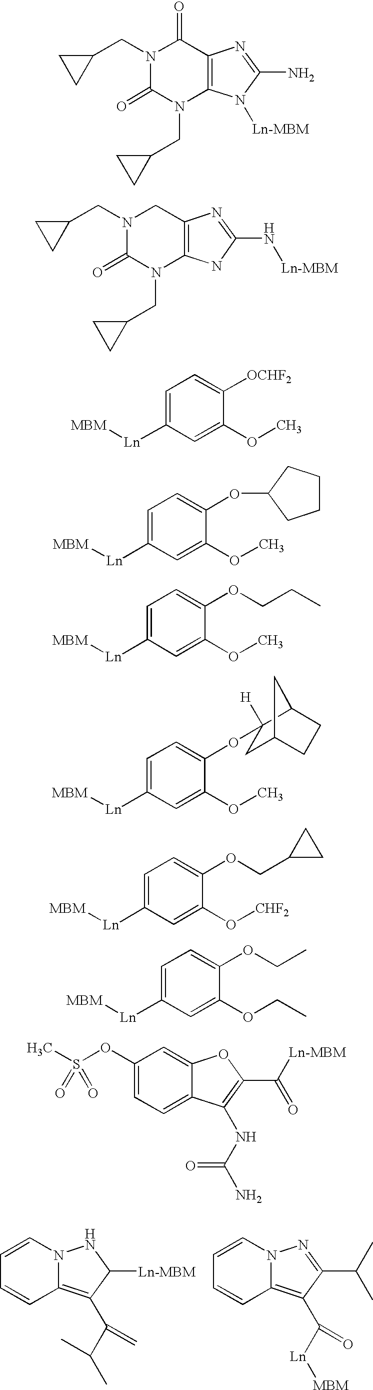 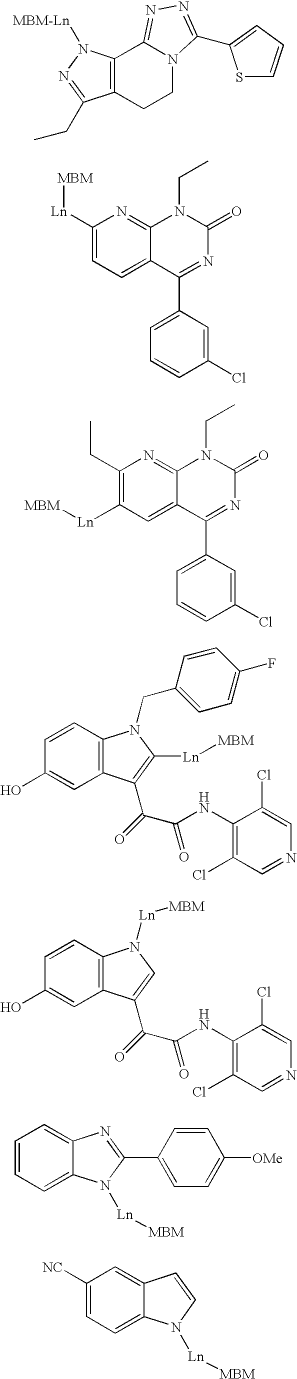 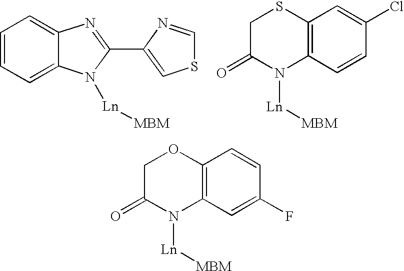
wherein
Ln is a linker;
n is 0 or 1; and
MBM is a metal binding moiety.

23. An inhibitor of a PDE4 enzyme having a formula selected from the group consisting of: 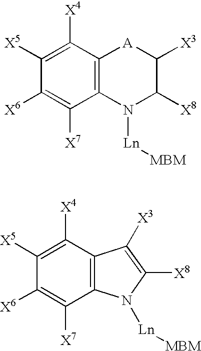
A is S, O, SO₂ or NX 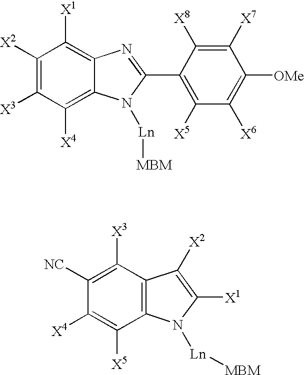
A is N or C, subject to the proviso that X⁵ is absent when A is N 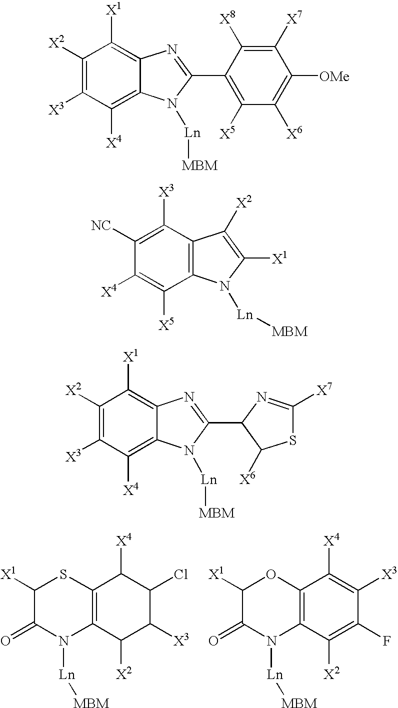
X, X₁-X₈ are independently hydrogen or a substituent
wherein
Ln is a linker;
n is 0 or 1; and
MBM is a metal binding moiety.

24. An inhibitor of an adenosine deaminase enzyme having a formula selected from the group consisting of: 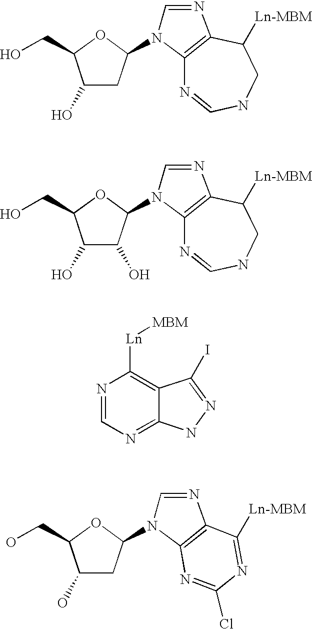
wherein
Ln is a linker;
n is 0 or 1; and
MBM is a metal binding moiety.

25. An inhibitor of an angiotensin converting enzyme having a formula selected from the group consisting of: 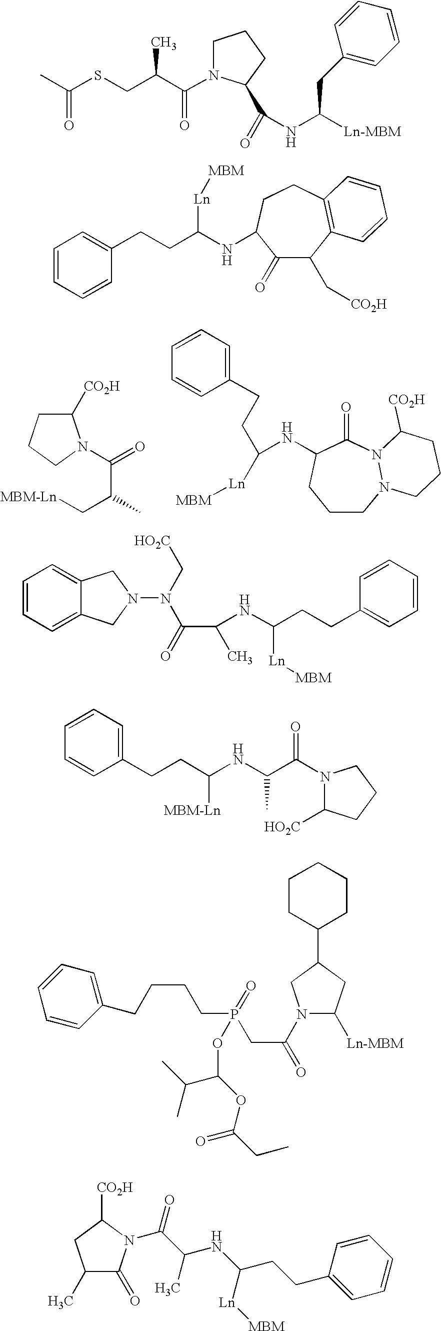 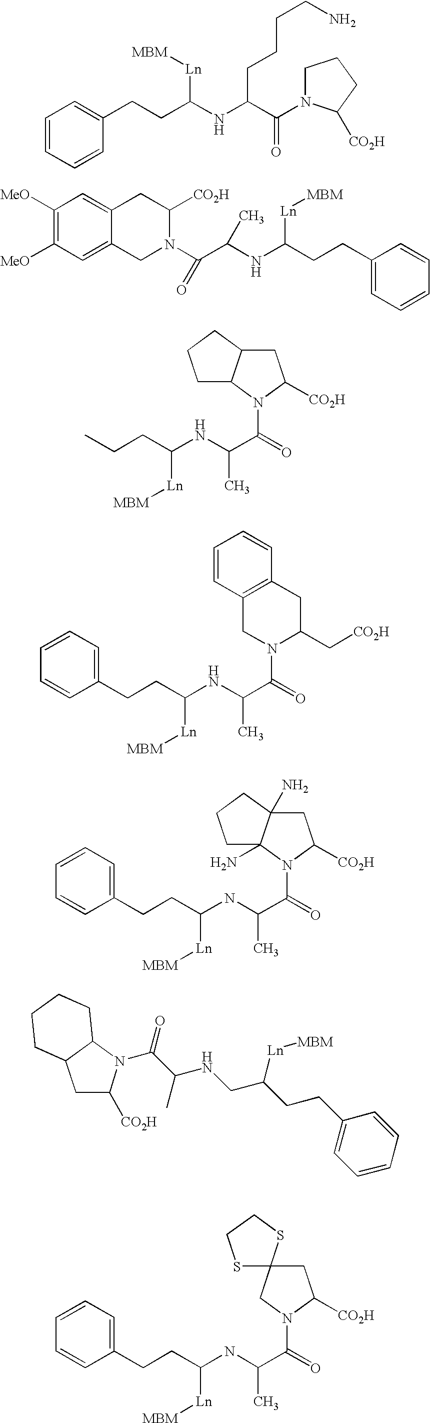 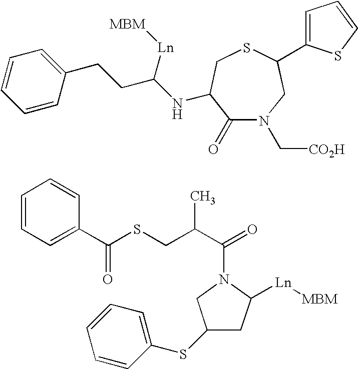
wherein
Ln is a linker;
n is 0 or 1; and
MBM is a metal binding moiety.

26. An inhibitor of a calcineurin enzyme having a formula selected from the group consisting of: 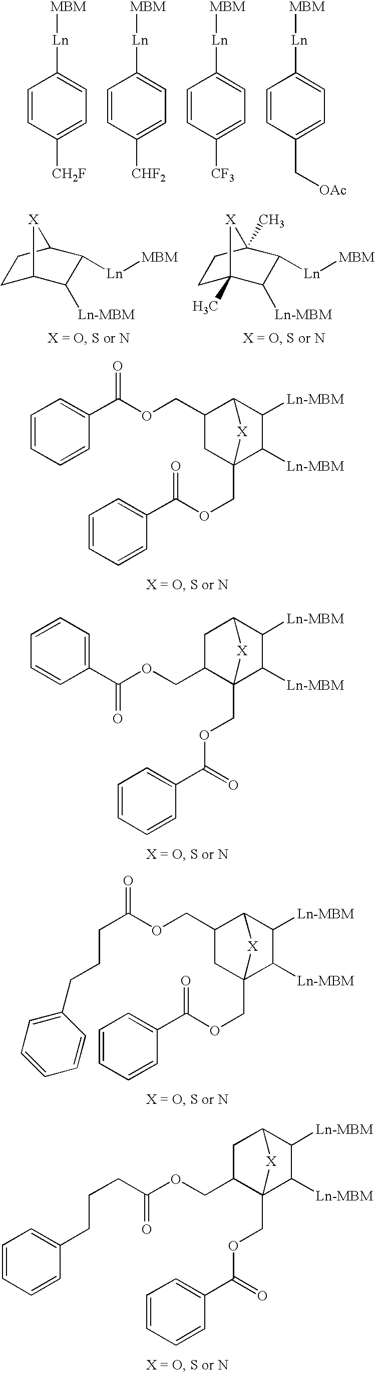 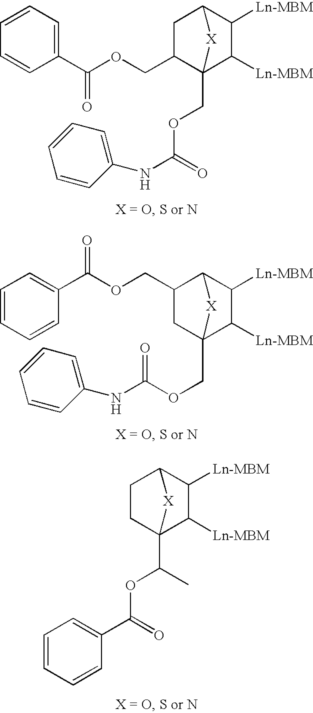
wherein
Ln is a linker;
n is 0 or 1; and
MBM is a metal binding moiety.

27. An inhibitor of a metallo-beta-lactamase enzyme having a formula selected from the group consisting of: 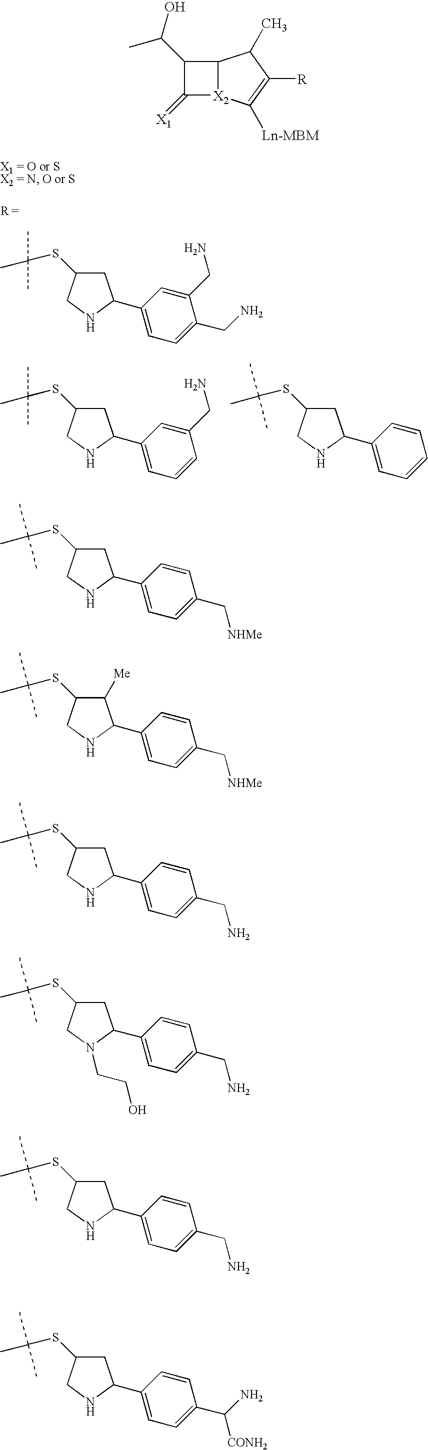 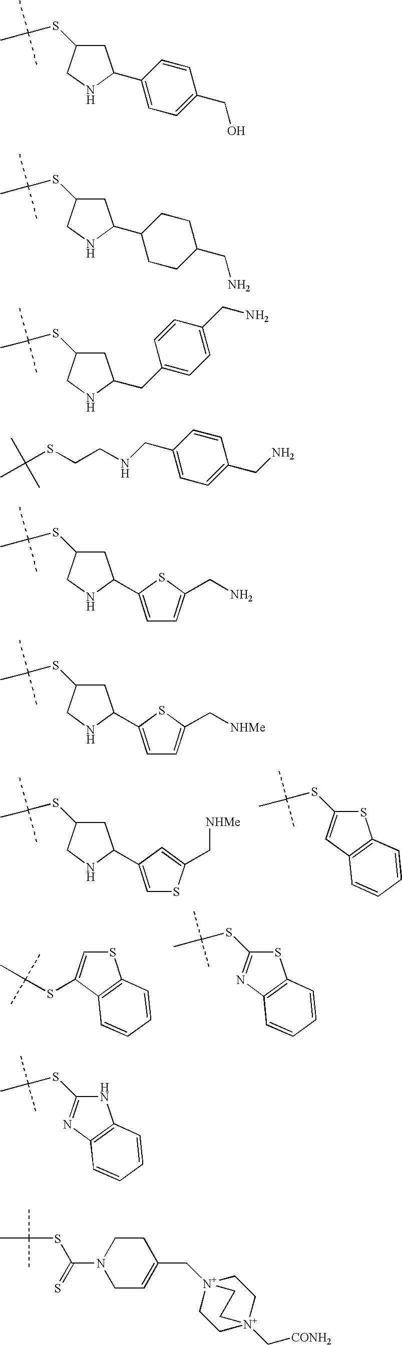 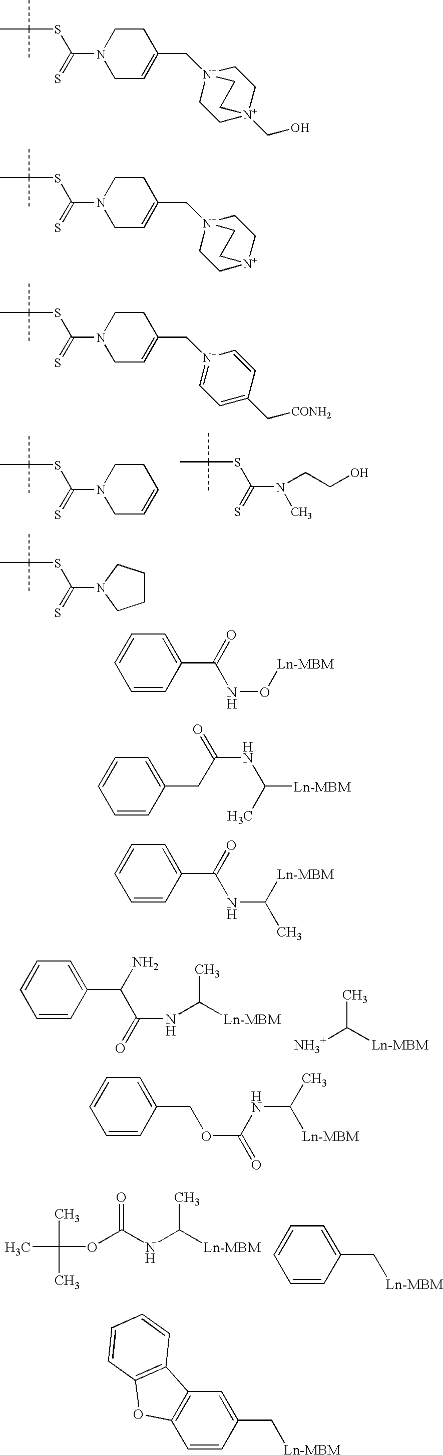 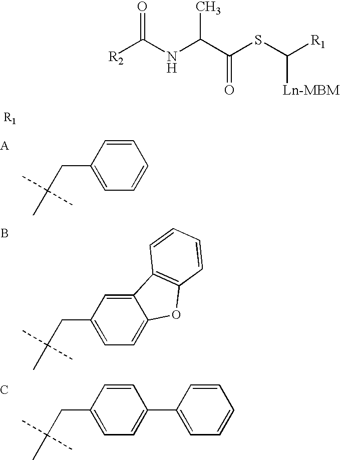
R₁, R₂ are selected from the following combination

| Compound | R₁ | R₂ |
|---|---|---|
| 1 | H | Ph— |
| 2 | A | CH₃— |
| 3 | A | H₂C=CHCH₂O— |
| 4 | A | Ph— |
| 5 | B | CH₃— |
| 6 | B | H₂C═CHCH₂O— |
| 7 | B | Ph— |
| 8 | C | CH₃— |
| 9 | C | H₂C═CHCH₂O— |
| 10 | C | Ph— |
| 11 | C | CH₃CH₂— |
| 12 | C | CH₃(CH₂)₂— |
| 13 | C | CH₃(CH₂)₃— |
| 14 | C | CH₃(CH₂)₄— |
| 15 | C | (CH₃)₂CH— |
| 16 | C | (CH₃)₂CHCH₂— |
| 17 | C | (E)-CH₃CH═CH— |
| 18 | C | HO₂C(CH₂)₂— |
| 19 | C | HO₂CCH₂SCH₂— |
| 20 | C | HO₂C(CH₂)₃— |
| 21 | C | PhCH₂— |
| 22 | C | PhOCH₂— |
| 23 | C | PhCH₂CH₂— |
| 24 | C | (E)-PhCH═CH— |
| 25 | C | PhCOCH₂CH₂— |
| 26 | C | PhCONHCH₂— |
| 27 | C | 4-HO—PhCH₂— |
| 28 | C | 4-MeO—PhCH₂— |
| 29 | C | 4-(Me₂N)—PhCH₂— |
| 30 | C | 2-BnO—PhCH₂— |
| 31 | C | (3-pyridyl)-CH₂— |
| 32 | C | (1-naphthyl)-CH₂— |
| 33 | C | 4-MeO—Ph— |
| 34 | C | 3 -MeO—Ph— |
| 35 | C | 3-(Me₂N)—Ph— |
| 36 | C | 2,4,6-(MeO)₃—Ph— |
| 37 | C | 2-naphthyl- |

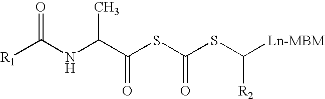
| R1 = | R2 = |
|---|---|
| 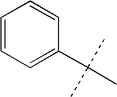 | H |
| 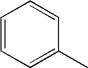 | 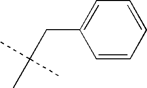 |
| 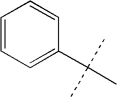 | 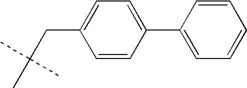 |
| 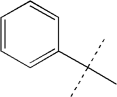 | 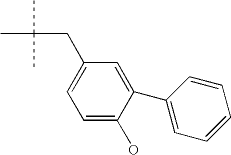 |
| 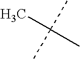 | 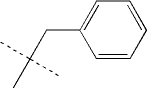 |
| 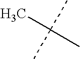 | 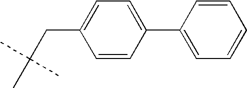 |
| 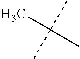 | 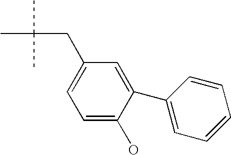 |

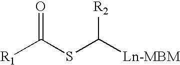
| R1 = | R2 = |
|---|---|
| 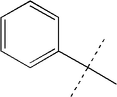 | 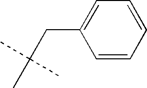 |
| 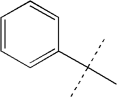 | 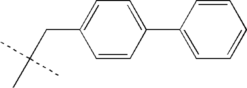 |
| 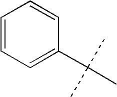 | 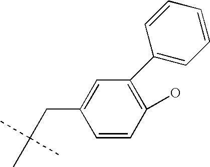 |
| 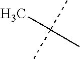 | 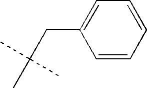 |
| 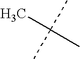 | 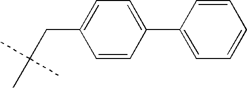 |
| 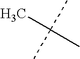 | 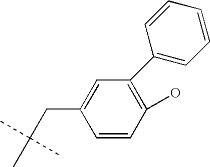 |
| 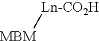 | 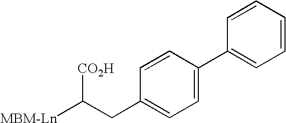 |
| 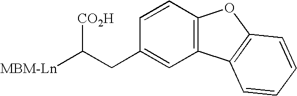 | 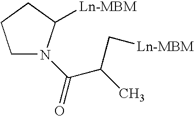 |

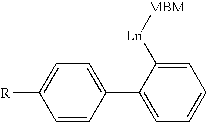

R =

| | |
|---|---|
| H | CH₃ |
| 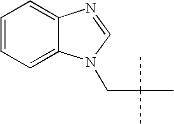 | 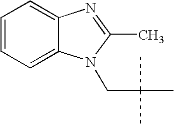 |
| 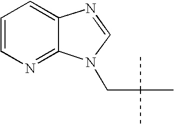 | 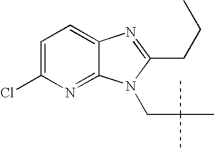 |
| 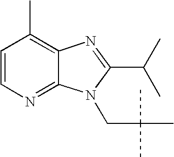 | 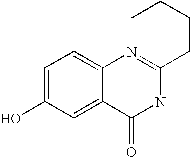 |
| 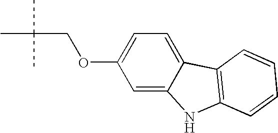 | 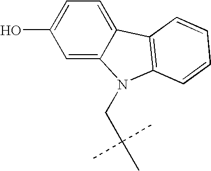 |
| 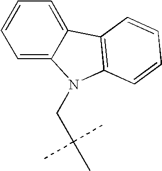 | 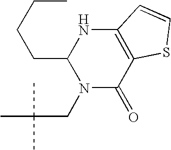 |
| 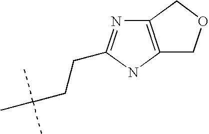 | |

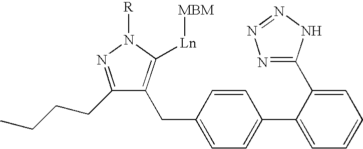

R═Ph, Ph(2-Cl), Ph(3-Cl), Ph(4-Cl), Ph(2,3-Cl₂), Ph(2,4-Cl₂), Ph(2,5-Cl₂), Ph(2,6-Cl₂), Ph(2,4,6-Cl₃), Ph(2-CH₃), Ph(2-CF₃), Ph(2-NO₂), Ph(2-Ph)

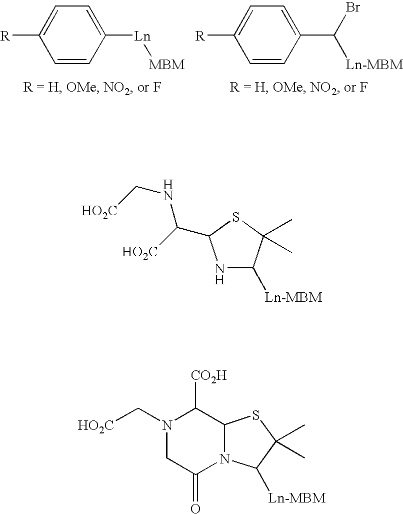

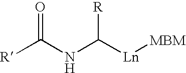

| R | R′ |
|---|---|
| CH₃ | PhOCH₂ |
| CH₃ | Ph |
| CH₃ | PhOCH₂ |
| PhCH₂ | PhOCH₂ |

wherein
Ln is a linker;
n is 0 or 1; and
MBM is a metal binding moiety.

28. An inhibitor of a PDE3 enzyme having a formula selected from the group consisting of: 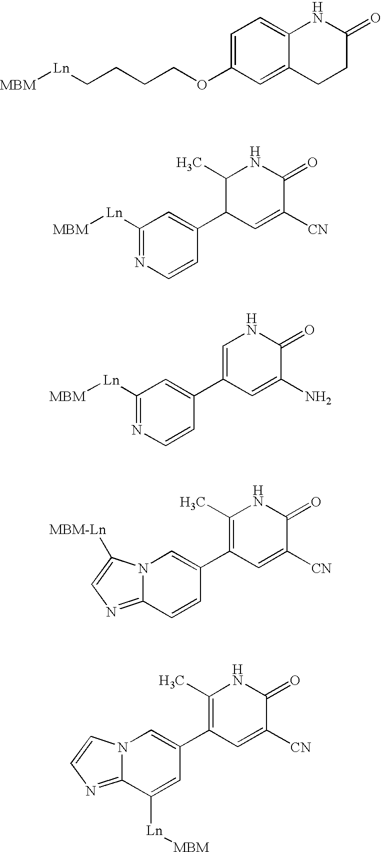
wherein
Ln is a linker;
n is 0 or 1; and
MBM is a metal binding moiety.

29. An inhibitor of a PDE5 enzyme having a formula selected from the group consisting of: 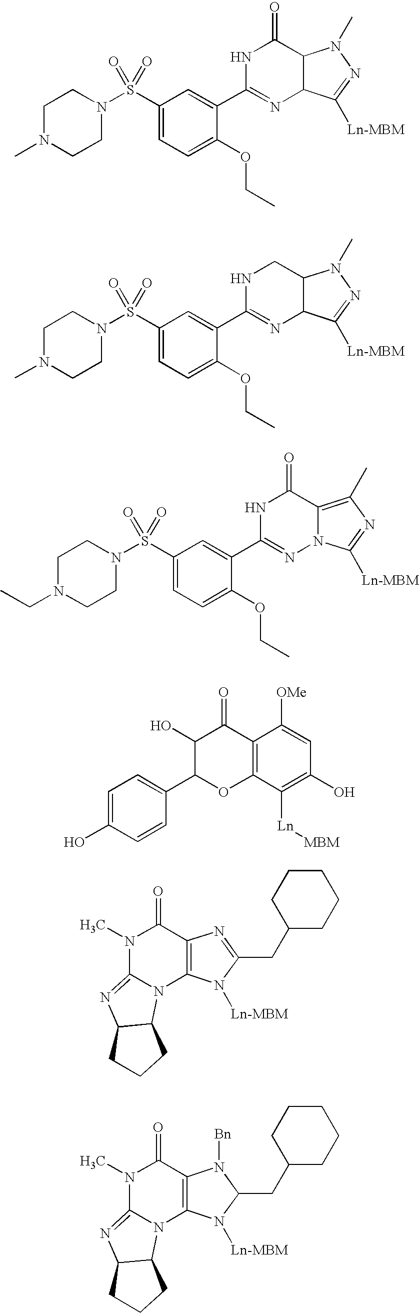 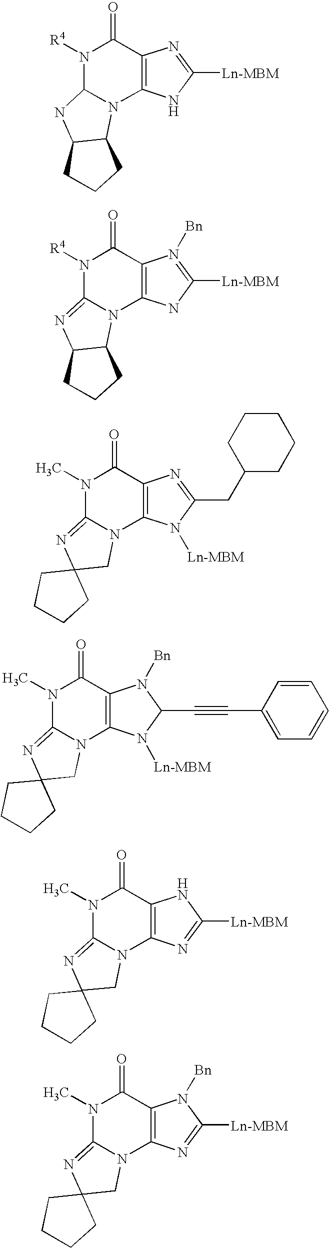 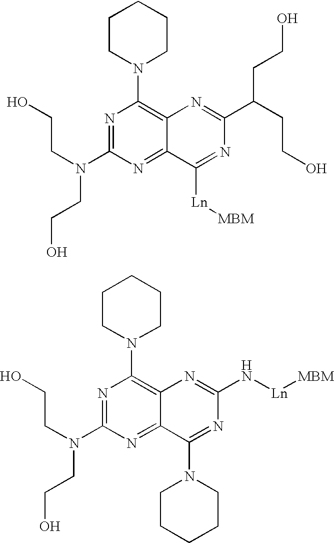
wherein
Ln is a linker;
n is 0 or 1; and
MBM is a metal binding moiety.

30. An inhibitor of a renal dipeptidase enzyme having a formula selected from the group consisting of: 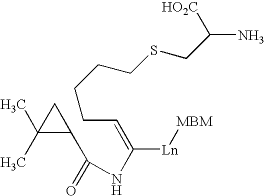
wherein
Ln is a linker;
n is 0 or 1; and
MBM is a metal binding moiety.

31. An inhibitor of a urease enzyme having a formula selected from the group consisting of: 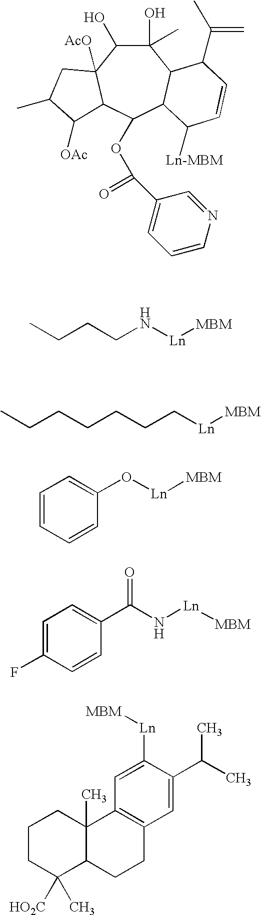
wherein
Ln is a linker;
n is 0 or 1; and
MBM is a metal binding moiety.

32. An inhibitor according to claim 22 wherein said MBM is selected from the group consisting of a sulfonyl moiety, a carbonyl moiety, a sulfur containing moiety, a nitrogen containing moiety, a phosphorus containing moiety, five membered aromatic rings with 1 heteroatom, five membered aromatic rings with 2 heteroatoms, five membered aromatic rings with 3 heteroatoms, five membered aromatic rings with 4 heteroatoms, five membered non-aromatic rings with 1 heteroatom, five membered non-aromatic rings with 2 heteroatoms, six membered aromatic rings with no heteroatoms, six membered aromatic rings with 1 heteroatom, six membered aromatic rings with 2 heteroatoms, six membered aromatic rings with 3 heteroatoms, six membered non-aromatic rings with 1 heteroatom, and six membered non-aromatic aromatic rings with 2 heteroatoms.

33. An inhibitor according to claim 22, wherein said MBM is selected from the group consisting of 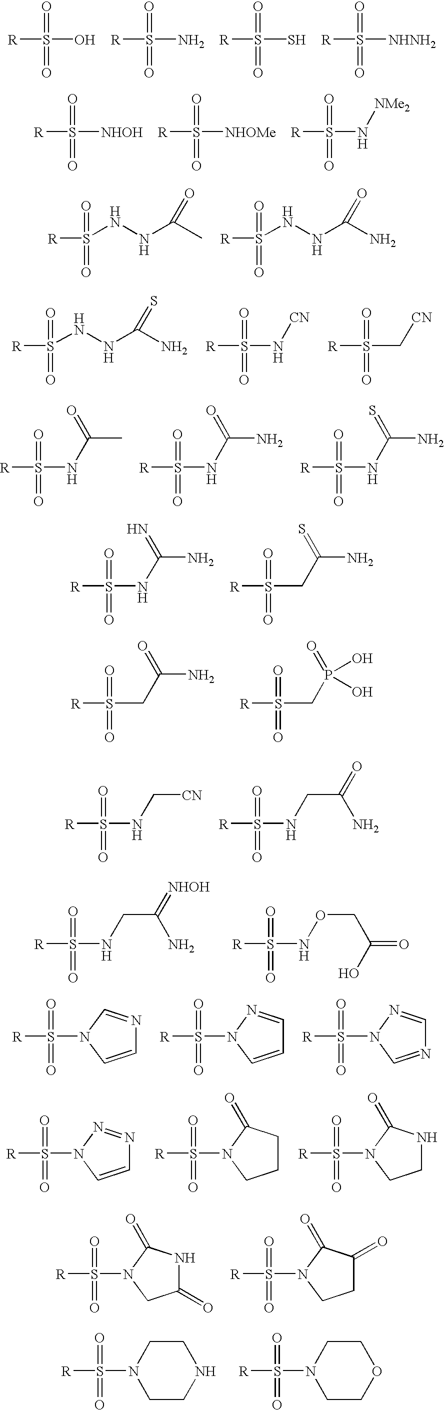 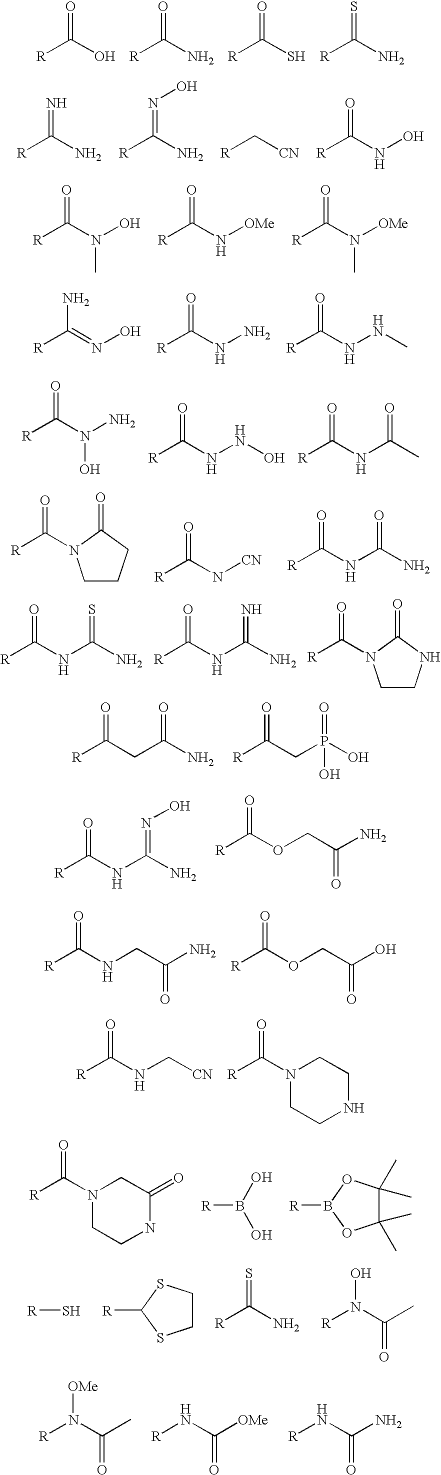 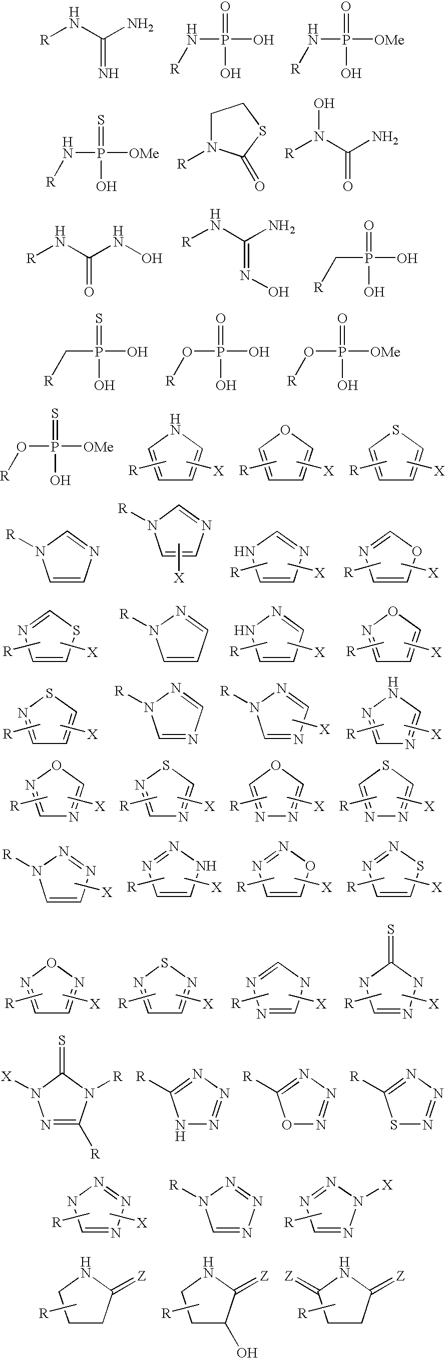 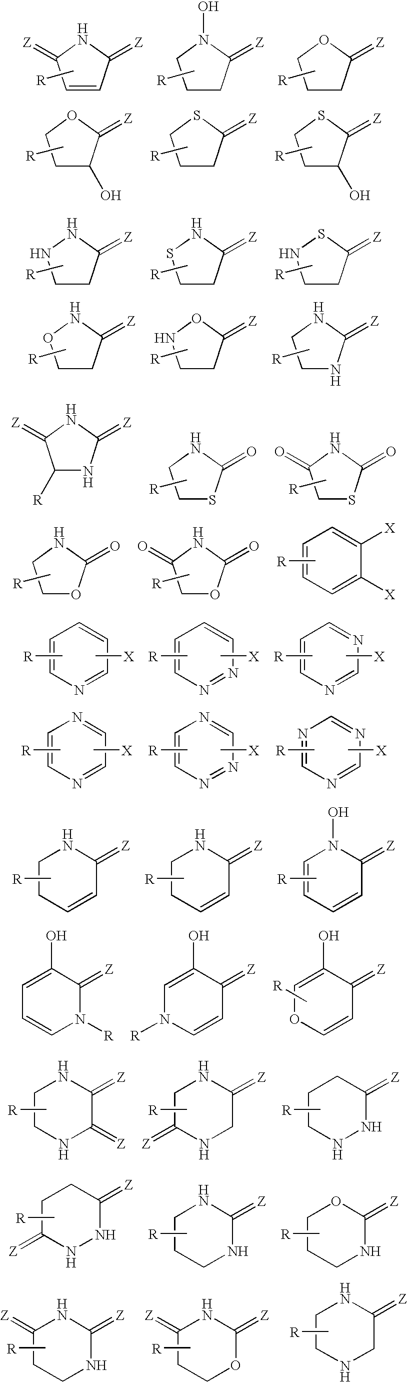 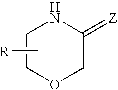
wherein
R is the point of attachment to the optional linker or MBM;
X is an optional substituent; and
Z is O or S.

34. An inhibitor according to claim 23 wherein said MBM is selected from the group consisting of: 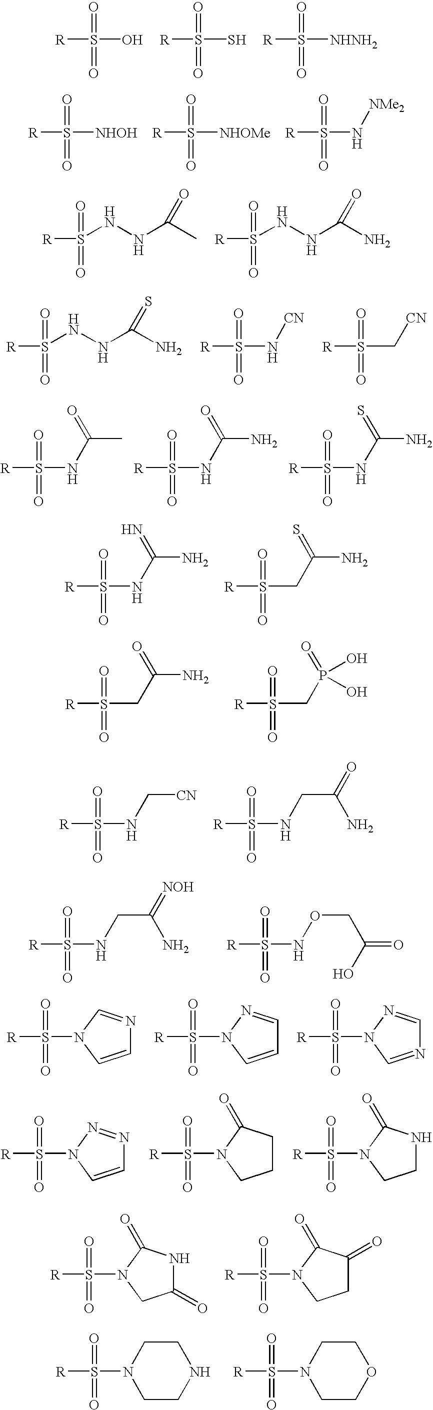 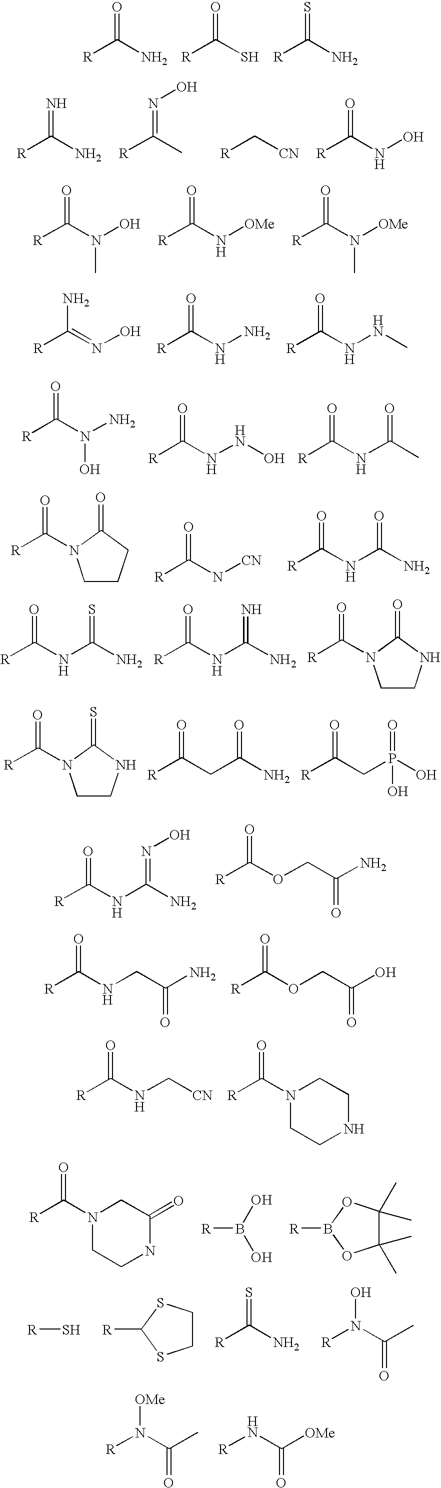 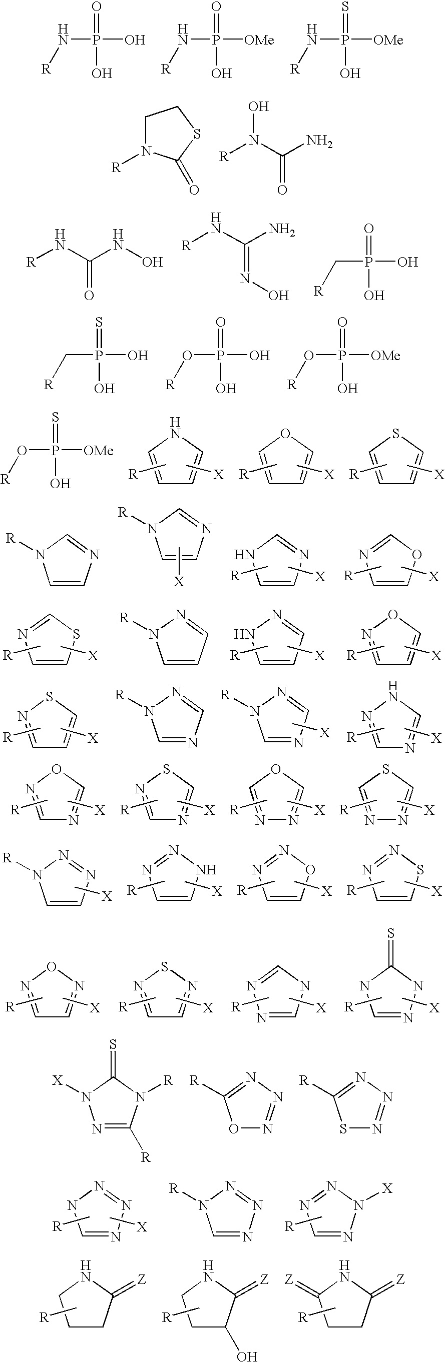 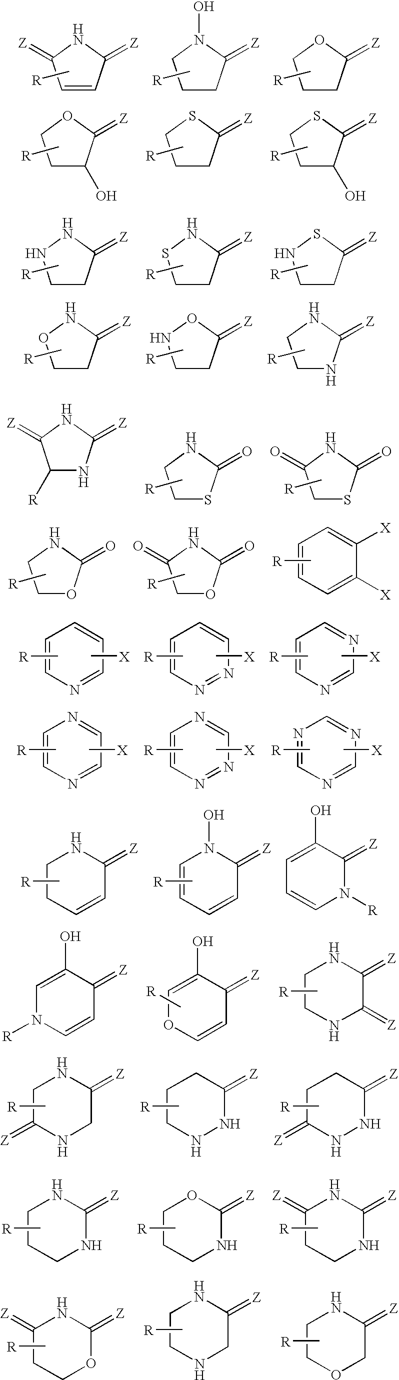
wherein
R is the point of attachment to the optional linker or MBM;
X is an optional substituent; and
Z is O or S.

35. An inhibitor according to claim 22 wherein said linker is a C1-C6 alkyl moiety.

36. An inhibitor according to claim 35 wherein said C1-C6 alkyl moiety is a substituted alkyl moiety.

37. An inhibitor according to claim 23 wherein said linker is a C3-C6 linear or branched alkyl moiety.

38. An inhibitor according to claim 37 wherein said C3-C6 alkyl moiety is a substituted alkyl moiety.

39. A pharmaceutical composition comprising an inhibitor of claim 1 and a pharmaceutical carrier.

40. A method of screening for inhibitors of a metallo-hydrolase comprising:
a) providing a candidate inhibitor comprising:
i) a targeting moiety;
ii) a metal binding moiety (MBM); and
iii) a linker;
b) contacting said inhibitor candidate with said metallo-hydrolase; and
c) determining the activity of said metallo-hydrolase.

41. A method of inhibiting a metallo-hydrolase comprising contacting said metallo-hydrolase with an inhibitor according to claim 1.

42. A method of treating a metallo-hydrolase related disorder comprising administering a composition according to claim 1, a prodrug, or a salt thereof.
